SEQUENCE LISTING (67 sequences)

SEQ ID NO: 1 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 2 - DNA, 11058 bp, Homo sapiens
  source 1..11058: mol_type = genomic DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagctgcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 3 - DNA, 5953 bp, synthetic construct
  misc_feature 1..5953: note = Synthetic
  source 1..5953: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttggcagcagtcagccacacaacgactggaacatgcattcaacatcgccagatatcaattaggcatagagaaagtactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagtacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaagtttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttagtaaggaaactgccatctccaaactagaaatgccatcttccttgatgttggaggtacctgctctggcagatttcaacggggcttggacagaacttaccgagtggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacagggcagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtcggtgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcaggtgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcgtccccaggacacaagcacagggttagaggaggtgatggagcaactcaacaactcctttccctagttcaagaggaagaaatacccctggaaagccaatgagagaggacacaatgtag

SEQ ID NO: 4 - DNA, 5749 bp, synthetic construct
  misc_feature 1..5749: note = Synthetic
  source 1..5749: mol_type = other DNA
atgcttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtgcgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatctttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatgggaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgcgagtggcagacaaatgtagatgtggcaaatgacttggccctgaaacttctgcgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatgacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctgattgactctctccaagatcacctcgagaaagtcaaggcacttcgaggagaaattgcgcctctgaaagagaacgtgagcgacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtctttaaaagtggcatcatttccctgtgtaaagcacatttggaagacaagtacagataccttttcaagcaagtggcaagttcaacaggattttgtgaccagcgcaggctgggcctccttctgcatgattctatccaaatccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctggagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttgaagaggaagaaatacccctggaaagccaatgagagaggacacaatgtag

SEQ ID NO: 5 - DNA, 6397 bp, synthetic construct
  misc_feature 1..6397: note = Synthetic
  source 1..6397: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacagcaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcgtattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactgagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctagagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagccggtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggtttagaggaggtgatggagcaactcaacaactccttccctagttcaagaggaagaaatacccctggaaagccaatgagagaggacacaatgtag

SEQ ID NO: 6 - DNA, 6718 bp, synthetic construct
  misc_feature 1..6718: note = Synthetic
  source 1..6718: mol_type = other DNA
atggtttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttcttcaacactgaagaataagtcagtgacccagaagacggaagcatggctggataactttgcccggtgttgggataatttagtccaaaaacttgaaaagagtacagcacagatttcacagtctgttgagaaatggcggcgttttcattatgatataaagatatttaatcagtggctaacagaagctgaagagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagtccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccgtggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaatttgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggatttcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctgagctttcacacgatgatactcattcacgcattgaacatatgctagcaggctagcagaaatggaaaacagcaatggatcttatctaaatgatagcatctctcctaatgagagcatagatgatgaacattttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacgtctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctcctttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 7 - DNA, 7049 bp, synthetic construct
  misc_feature 1..7049: note = Synthetic
  source 1..7049: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactgttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtatttgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgkaatgttatcaagtcttcaaaaagtggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctggccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgcgaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtgaacgtgcctctctgcgtggatatgtgtctgaactggctggtgaatgtttatgatacgggacgaacagggaggatccgtgtcctgtcttttaaaactggcatcatttccctgtgtaaagcacatttggaagacaagtacagataccttttcaagcaagtgggaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacaggatgatactcattcacgcattgaacattatgctagcaggctagcagaaatggaaaacagcaatggatctttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctggtggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 8 - DNA, 6398 bp, synthetic construct
  misc_feature 1..6398: note = Synthetic
  source 1..6398: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggcttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaaggctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacagcctgacctagctcctggactgacgactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctgtgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaaggagttggccaaagacctccgccagtggcagacaaatgtagatgtggcaaatgacttggccctgaaacttgtccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaatgaagctcacacagatgtttatcacaacctggatgaaaacagccaaaaaatcctgagatccctggaaggttccgatgatgcagtcctgttacaagacgthtggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcatgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcgtggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 9 - DNA, 6078 bp, synthetic construct
  misc_feature 1..6078: note = Synthetic
  source 1..6078: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaagaatgtcaacaaggcactgcgggttttgcagaacaataatgttgatttagtgaatattggaagtactgagatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgtgggataatttagtccaaaaacttgaaaagagtacagcacagatttcacaggaaattcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagacagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactctgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttggggattcagctctcaccgtataacctcagcactctggaagacctgaacaccagatggaagcttctgcaggtggccgtcgaggacggagtcaggcaggtgcatgaagcccacagggactttggtccagcatgtcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaagcaaaaggtattttgcgaagcatccccgaatgggctacctgccagtgcagactgtcttagagggggacaacatggaaacgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttgaagaggaagaaatacccctggaaagccaatgagagaggacacaatgtag

SEQ ID NO: 10 - DNA, 6264 bp, synthetic construct
  misc_feature 1..6264: note = Synthetic
  source 1..6264: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatgatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaatttatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggacggacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactctgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 11 - DNA, 4078 bp, synthetic construct
  misc_feature 1..4078: note = Synthetic
  source 1..4078: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccagaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttcgaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacgtgagaaagaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagtcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagctaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgagcactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 12 - DNA, 3761 bp, synthetic construct
  misc_feature 1..3761: note = Synthetic
  source 1..3761: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggtaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacgaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 13 - DNA, 3434 bp, synthetic construct
  misc_feature 1..3434: note = Synthetic
  source 1..3434: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaattacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggacgatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 14 - DNA, 3439 bp, synthetic construct
  misc_feature 1..3439: note = Synthetic
  source 1..3439: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttggcagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcatgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcgcatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacgaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagtcaacaggattttgtgaccagcgcaggctgggcctccttctgcatgattctatccaaattccaagacagttgggtgaagttgcatcctttgggggcagtaacattgaggcaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtag

SEQ ID NO: 15 - DNA, 8312 bp, synthetic construct
  misc_feature 1..8312: note = Synthetic
  source 1..8312: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctgaccggctccagatttatcagcaataaaccagccagccggaagggccgagcgcagaagtggtcctgcaagtttatccgcctccatccagtgtattaattgttgccgggaagctagagtaagtagttcgccagttaatagtttgcgcaacgttgttgccattgctacaggcatcgtggtgtcaggctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagtcgatgtaacccactcgtgcacccaactgatcttcagcatcttttactttcaccagcgtttctgggtgagcaaaaacaggaaggcaaaatgccgcaaaaaagggaataagggcgacacggaaatgttgatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaagctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgtaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcaggtataacgtgctttcctcgttagaatcagagcgggagctaaacaggaggccgattaaagggattttagacaggaacggtacgccagaatcctgagaagtgtttttataatcagtgaggccaccgagtaaaagagtctgtcgatcacgcaaattaaccgttgtcgcaatacttctttgattagtaataacatcacttgcctgagtagaagaactcaaactatcggccttgctggtaatatccagaacaatattaccgccagccattgcaagaggaaaaacgctcatggaaatacctacattttgacgctcaatcgtctggaattccattcgccattcaggctgcgcaactgttgggaagggcgatcggtgcgggcctcttcgctattacgccagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacggccgcggtaccgcgttacataacttacggtaaatggcccgcctggctgaccgcccaacgacccccgcccattgacgtcaataatgacgtatgttcgcatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtctagacggccgcggttttttttatcgctgccttgatatacactttccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggggcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcgcatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctccgagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagagacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgaggtggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtaggaagtcttttccacatggcagatgatttgggcagagcgatggagtccttagtatcagtcatgacagatgaagaaggagcagaataaatgttttacaactcctgattcccgcatgcggccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttgcggccgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctg

SEQ ID NO: 16 - DNA, 8293 bp, synthetic construct
  misc_feature 1..8293: note = Synthetic
  source 1..8293: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctgagcgatctgtctatttcgttcatccatagttgcctgactccccgtcgtgtagataactacgatacgggagggcttaccatctggccccagtgctgcaatgataccgcgagacccacgctgaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaataggggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttcttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaattccattcgccattcaggctgcgcaactgttgggaagggcgatcggtgcgggcctcttcgctattacgccagctggcgcgctcgctcgctcactgaggccgccggggcaaagcccgggcgtcgggcgacctttggtcgcccggcctcagtgagcgagcgagcgcgcagagagggagtggccaactccatcactaggggttccttgtagttaatgattaacccgccatgctacttatctacggccgcggtaccactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtctctagacggccgcggttttttttatcgctgccttgatatacactttccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaagttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacggcaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacgaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcgtcaaaaacagatgccagtattctacaggaaaaattgggaagcctgaatctgcggtggcaggaggtctgcaaacagctgtcagacagaaaaaagaggctagaagaaacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcaggtctcacggtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccgcagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtaggaagtcttttccacatggcagatgatttgggcagagcgatggagtccttagtatcagtcatgacagatgaagaaggagcagaataaatgttttacaactcctgattcccgcatgcggccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttgcggccgtagataagtagcatggcgggttaatcattaactacaaggaacccctagtgatggagttggccactccctctctgcgcgctcggtcgctcactgaggccgggcgaccaaaggtcgcccgacgcccgggctttgcccgggcggcctcagtgagcgagcgagcgcgcagctgctg

SEQ ID NO: 17 - DNA, 756 bp, Homo sapiens
  source 1..756: mol_type = genomic DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatggcctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaa

SEQ ID NO: 18 - DNA, 252 bp, Homo sapiens
  source 1..252: mol_type = other DNA
atgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggag

SEQ ID NO: 19 - DNA, 333 bp, Homo sapiens
  source 1..333: mol_type = other DNA
agtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacataga

SEQ ID NO: 20 - DNA, 327 bp, Homo sapiens
  source 1..327: mol_type = other DNA
gttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagac

SEQ ID NO: 21 - DNA, 333 bp, Homo sapiens
  source 1..333: mol_type = other DNA
atccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacag

SEQ ID NO: 22 - DNA, 150 bp, Homo sapiens
  source 1..150: mol_type = other DNA
gctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggat

SEQ ID NO: 23 - DNA, 262 bp, Homo sapiens
  source 1..262: mol_type = other DNA
tctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcct

SEQ ID NO: 24 - DNA, 389 bp, Homo sapiens
  source 1..389: mol_type = other DNA
cagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagaca

SEQ ID NO: 25 - DNA, 333 bp, Homo sapiens
  source 1..333: mol_type = other DNA
atttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggag

SEQ ID NO: 26 - DNA, 327 bp, Homo sapiens
  source 1..327: mol_type = other DNA
caaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaaggga

SEQ ID NO: 27 - DNA, 327 bp, Homo sapiens
  source 1..327: mol_type = other DNA
ggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaa

SEQ ID NO: 28 - DNA, 312 bp, Homo sapiens
  source 1..312: mol_type = other DNA
gtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacag

SEQ ID NO: 29 - DNA, 288 bp, Homo sapiens
  source 1..288: mol_type = other DNA
agcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaa

SEQ ID NO: 30 - DNA, 315 bp, Homo sapiens
  source 1..315: mol_type = other DNA
ccagccaattttgagctgcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaa

SEQ ID NO: 31 - DNA, 324 bp, Homo sapiens
  source 1..324: mol_type = other DNA
tgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaa

SEQ ID NO: 32 - DNA, 306 bp, Homo sapiens
  source 1..306: mol_type = other DNA
taccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccatt

SEQ ID NO: 33 - DNA, 288 bp, Homo sapiens
  source 1..288: mol_type = other DNA
cctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagagagaggaaataaagataaaacagcagctgttacagacaaaacataatgctctcaaggatttgaggtctcaaagaagaaaaaaggctctagaa

SEQ ID NO: 34 - DNA, 297 bp, Homo sapiens
  source 1..297: mol_type = other DNA
atttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaa

SEQ ID NO: 35 - DNA, 384 bp, Homo sapiens
  source 1..384: mol_type = other DNA
attcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacaga

SEQ ID NO: 36 - DNA, 321 bp, Homo sapiens
  source 1..321: mol_type = other DNA
tctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaa

SEQ ID NO: 37 - DNA, 330 bp, Homo sapiens
  source 1..330: mol_type = other DNA
caaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagct

SEQ ID NO: 38 - DNA, 315 bp, Homo sapiens
  source 1..315: mol_type = other DNA
caaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaag

SEQ ID NO: 39 - DNA, 141 bp, Homo sapiens
  source 1..141: mol_type = other DNA
cagcctgacctagctcctggactgaccactattggagcctctcctactcagactgttactctggtgacacaacctgtggttactaaggaaactgccatctccaaactagaaatgccatcttccttgatgttggaggtacct

SEQ ID NO: 40 - DNA, 321 bp, Homo sapiens
  source 1..321: mol_type = other DNA
gctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaa

SEQ ID NO: 41 - DNA, 327 bp, Homo sapiens
  source 1..327: mol_type = other DNA
atgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaa

SEQ ID NO: 42 - DNA, 348 bp, Homo sapiens
  source 1..348: mol_type = other DNA
actcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagcc

SEQ ID NO: 43 - DNA, 387 bp, Homo sapiens
  source 1..387: mol_type = other DNA
agttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgag

SEQ ID NO: 44 - DNA, 327 bp, Homo sapiens
  source 1..327: mol_type = other DNA
acccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaa

SEQ ID NO: 45 - DNA, 216 bp, Homo sapiens
  source 1..216: mol_type = other DNA
gcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactc

SEQ ID NO: 46 - DNA, 888 bp, Homo sapiens
  source 1..888: mol_type = other DNA
cgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaact

SEQ ID NO: 47 - DNA, 834 bp, Homo sapiens
  source 1..834: mol_type = other DNA
cccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaacccgaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtag

SEQ ID NO: 48 - DNA, 11044 bp, Canis lupus
  source 1..11044: mol_type = genomic DNA
atgctttggtgggaagaagtagaggactgttatgaaagagaagatgttcaaaagaaaacattcacaaaatgggtaaatgcacagttttctaagtttgggaagcagcacatagagaacctcttcagtgacctacaggatgggagacgcctcctagaccttttggaaggcctgacagggcaaaaactgccaaaagaaaaaggatccacaagagttcatgccctgaacaatgtcaacaaggcactgcgcgtcttgcagaaaaataatgttgatttagtgaacattggaagtactgacatagtagatggaaatcacaaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtcattaacttcaccaccagctggtctgatggcctggctttgaacgctctcatccacagtcataggccagacctgtttgattggaatagtgtggtttgccagcagtcagccacacaacgcctggaacatgcattcaacattgccaaatatcaattaggcatagagaaactgcttgatcctgaagatgttgccaccacttatccagataagaagtccatcttaatgtatatcacatcactcttccaagttttgcctcaacaagtgagcattgaagccatccaggaagtggaaatgttgccaaggccatctcaagttactagagaagaacattttcagatacatcatcaaatgcactattctcaacagatcacagtcagtctagcacagggatatgaacgagccccttcctttcctaagcctcggttcaagagctatgcctacacacaggctgcttatgtcaccacttctgaccccacacggagcccacttccttcacagcatttggaaactcctgaagacaagtcatttggccggtcattgacagagaccgaagcaaacctggacagttatcaaacagctttggaagaagtactctcgtggcttctttcagctgaggatgcactgcaagcccaaggagagatttctaatgatgtcgaagaagtgaaagaacaatttcatactcatgagggatatatgatggacttgacatcccatcagggacgggtcggtaatgttctccaactgggaagtcaactgattggaacagggaaattatcagaagatgaagaaaccgaagtgcaggaacaaatgaatctcctcaattcaagatgggaatgcctcagggtagctagcatggaaaaacaaagcaatttacataaagttctaatggatctccagaatcagcaactgaaagagttaaatgactggctaaccaaaacagaagagagaacaaggaaaatggagaaggagccccttggacctgatattgaagacctaaaacgccaagtacaacaacataaggtgcttcaagaagacttagaacaggaacaagtcagggtcaattccctcactcatatggtggtggtagtcgatgaatctagtggagaccatgcaactgctgctttggaagaacaacttaaggtactgggagatcgatgggcaaacatctgtaggtggacagaagatcgctgggttcttttacaagacatcctcctaaaatggcagcgttttactgaagaacagtgcctttttagtgcatggctttcggagaaggaagatgcagtgaacaagattcacacaactggctttaaggatcaaagtgaagtgttatcaaatcttcagaaactggctgtcttaaaaacagatctggaaaagaagaagcaaaccatggacaaactctgctcactcaaccaagaccttctttcagcgctgaaaaacacagtggtagcccacaagatggaagcatggctggacaactttgcccagcgctgggataatttagtccagaaacttgaaaaaagttcagcacagatttcacaggctgtcaccaccactcagccatcactaacacagacaactgtaatggaaacagtaactatggtgaccacgagggaacacatcttggtaaagcatgcccaagaggaactgccaccaccaccccctcagaagaagaggcagattatcgtggattctgaaattaggaaaaggttggatgtcgatataactgaacttcacagttggattactcgttcagaagctgtgttgcagagtcctgaatttgcaatctatcggaaggaaggcaacttctcagaccttaaagaaaaagtcaatgccatagagcgagaaaaagccgagaagttcagaaaactgcaagatgccagcagatcagctcaggccctggtggaacagatggtgaatgagggtgttaatgctgacagcatcaaacaagcctccgaacaactgaacagccggtggatagagttctgccaattgctaagcgagagacttaactggctggagtatcagaacaacatcatcactttctataatcagctacaacaattggagcagatgacaactactgctgaaaactggttgaaaacccagcctaccaccacatcagagccaacagcaattaaaagccagttaaaaatttgtaaggatgaaatcaaccgactgtcagctcttcagcctcaaatcgagcgattaaaaattcaaagcatagccctgaaagagaaaggacaagggccaatgttcctggatgcagactttgtggcctttacaaatcattttaaccaagtctttgctgatgtgcaggcaagagaaaaagagctacaaacaatttttgacagtttgccacccatgcgctatcaggagactatgagtaccatcctgacatggatccagcagtcagaaaccaaactctctatacctcaggttactgtcactgaatatgacatcatggaacagagactcggagagctacaggctttacaaagctctctgcaagagcaacaaaatggcctaaactatctcagcaccactgtgaaagagatgtcaaagaaagcaccactgtctgatattagtcggaaatatcaatcagaatttgaagagattgagggacgttggaagaagctgtcttcccagctggttgaacattgtcaaaagttggaggagcaaatggctaaacttcgaaaaattcagaatcacataaaaactctgaagaaatggatcactgaagtcgatgttttcctgaaggaggaatggcctgcccttggggattcagaaattctgaaaagacagctgaaacagtgcaggcttttagtcaatgacattcagaccatccagcctagtctcaacagtgtcaatgaaggggctcagaagatgaagaatgaagcagaaccagagtttgctggcagacttgagacagagctccgagaacttaacacccagtgggattacatgtgccgccaggtctatgccaggaaggaagccttaaaaggaggttttggataaaactgtaagtcttcagaaagatctgtcagagatgcatgagtggatgacacaagctgaagaagaatacctagagagagatttcgaatacaagacccctgatgaattacagacagcagttgaagagatgaagagagctaaagaagaggcccagcaaaaagaagcaaaagtgaaactcctaaccgagtccgtcaatagtgtcatagctcaggctccacctgcagcacaagaggccttaaaaaaggaacttgacactctcaccaccaactaccagtggctctgcaccaggctcaatggcaaatgcaagaccttggaagaagtttgggcgtgctggcatgagttattgtcctacttggagaaggcaaacaagtggctaagtgaagtagaagtcaagcttaaaaccactgaaaatatttctgggggagctgaggaaatcgccgaggtgcttgattcgcttgaaaatttgatgcaacattcagaggataacccgaatcagattcgcatattggcacagaccttgacagatggtggagtcatggatgaactgatcaatgaggagcttgagacatttaattctcgttggagagaactccatgaagaggctgtgaggaggcaaaagttgcttgagcagagtatccagtcggcccaggagatagaaaaatccttgcacttaattcaggagtccctctcttccattgacaagcagttggcagcttatattgctgacaaagtggatgcagctcagatgcctcaggaagcccagaaaatccaatcagatttgacaagtcatgagatcagtttagaagaaatgaagaaacataaccagggaaaggagactgcccaaagggtactatcccaaattgatgtggcacagaaaaaattgcaggatgtttccatgaagtttcgattattccagaagccagccaattttgagcagcgcctacaagaaagtaaaatgattttagatgaagtgaagatgcatttacctgcgttggaaacaaagagtgtggaacaggaagtagtacagtcacagttaaatcattgtgtgaacttgtataaaagtctgagtgaagtgaagtctgaagtggaaatggtaataaaaactggacgtcagattgtacagaagaagcagacggaaaacccgaaagagcttgatgaaagagttacagctttgaaattgcattataatgagctgggagcaaaggtgacagaaagaaagcaacagttggaaaaatgcttgaaattgtcccgtaagatgcgaaaggaaatgaatgccctgacagaatggctggcagctacagatatggaactgacaaagagatcggcagttgaaggaatgcctagtaatttggattctgaagttgcctggggaaaggctactcagaaagagattgagaaacagaaggttcacctaaagagtgtcacagaggtaggagaggccttgaaaacggttttgggcaagaaggaaatgttggtggaagataaactgagtcttctgaatagtaactggatagccgtcacttcccgagcagaagagtggttaaaccttttattggaataccagaaacacatggaaacttttgaccagaatgtggattacatcacaaactggatcattcaggctgatgcacttttggatgaatctgagaaaaagaaacctcagcaaaaagaagacatacttaagcgtttaaaggctgaaatgaatgacatacgtccaaaggtggattctacacgtgaccaagcagcaaacctgatggcaaaccgcggcgaccactgcaggaaagtagtagagcccaaaatctcagagctcaaccatcgatttgcagccatttctcacagaattaagactggaaaggcctccattcctttgaaggaattggagcagtttaactcagatatacaaaaattgcttgaaccactggaggctgaaattcagcagggggtgaatctgaaagaggaagacttcaataaagatatgagtgaagacaatgagggtactgtaaaagaattgttgcaaagaggagacaacttacaacaaagaatcacagatgagagaaagcgagaggaaataaagataaaacaacagctgttacagacaaaacataatgctctcaaggatttgaggtctcaaagaagaaaaaaggctctagaaatttctcaccagtggtatcagtacaagaggcaggctgatgatctcctgaaatgcttggatgacattgaaaaaaaattagccagcctacctgaacccagagatgaaaggaaaataaaggaaattgatcgtgaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccatgaagagtcagtggtggcgatgactgaagacatgcctttggaaatttcttatgtgccttctacttacctgactgagatcactcatgtctcacaagccctatcagaagtggaagagcttcttaatgctcccgacctttgtgctcaagattttgaagatctctttaaacaagaggaatccttgaagaacataaaagacagcctgcaacaaatctcaggtcggattgacatcattcacaataaaaagacagcagcattgcacagtgccactcctgcagaaagggcaaagctccaggaagctctctcacggcttgatttccaatgggaaagagttaacaatatgtacaaggaccgacaagggagatttgacagatctgtggaaaaatggcggcggtttcattatgatatgaagatacttaatcaatggctaacagaagctgaacagtttctcaaaaagacacaaattcctgagaattgggaacatgccaaatacaaatggtatcttaaggaactccaggatggcattggacagcggcaaagtgttgtcagggtattgaatgcaactggggaagaaataattcaacagtcctcaaaaacagatgccagtattctccaagaaaaactgggaagcctgaatctgcggtggcaggaggtctgcaaacagctggcagaaagaaaaaagaggctagaggaacagaagaatatcttgtcagaatttcaaagagatgtaaatgaatttgttttatggttggaagaagcggataacgttgctaatattccacttgaacctggaaatgagcagcagctaaaagaaaaacttgaacaagtcaagttactggcagaagagttgcccctgcgccagggaattctaaaacaattaaatgaaactggaggaacagtgcttgtaagtgctcccctaagcccagaagagcaagataaacttgaaaataagctcaagcagacaaatcttcagtggataaaggtttctagaaatctgcctgagaagcaagaagaaattgaggcacacgtaaaagaccttggacagctggaagagcagttaaatcatctgcttctatggctgtctcctattaggaatcagttggaaatttacaatcagccaaatcaaacaggaccatttgacatcaaggaaattgaagtagcagttcaagctaaacagccggatgtggaagggattttgtctaaagggcagcatttgtacaaggaaaaaccagccactcagccagcgaagagaaagctggaagatctcagctctgattggaaggtggtaactcagttgcttcaagagctgcgggcaaagcaacctggcccagctcctggactgaccactgtcagagcccctcccagtcagactgttactctggtgacacaacccgcggttaccaaggaaactgccatctccaaactagaaatgccatcttcattgctgttggaggtacctgcactggcagatttcaaccgagcttggacagaacttaccgactggctgtctctgcttgatcgagttataaaatcacagagggtgatggtgggtgatcttgaagacattaacgagatgatcatcaagcagaaggcaacgctgcaggatttggaacagaggcgcccccagttggaagaactcattaccgctgcccagaatttgaaaaacaagaccagcaatcaagaggctagaacaatcattactgatcgaattgaaagaattcagagtcagtgggatgaagtacaggaacatcttcagaaccggaggctacagttgactgaaatgttaaaggattccacacaatggctggaagctaaagaggaggctgagcaggtgttggggcaggccagagccaagcttgagtcatggaaggaggctccctacacagtagatgcaatccaaaagaaaatcacagaaaccaagcagttggccaaagacctccgccagtggcagataaatgtagatgttgcaaatgatttggcactgaaacttctccgagattattctgcagatgataccagaaaagtacacatgataacagagaacatcaatgcctcttgggcaagcatccataaaagattgagtgagcgagaggctgctctggaagaaacccacagattactgcaacagttccccttggacctggagaagttccttgcctggcttacagaagccgaaacaactgccaacgtcctgcaggatgccacccataaggaaaggcttctagaagattccaagggagtaagagagctgatgaaacaatggcaagacctccaaggagaaatcgaagctcacacagatatctatcacaacctggacgaaaatggccaaaaagtcctgagatccctggaaggttctgacgatgcagccttgttgcaaagacgtttggataacatgaacttcaagtggagcgaacttcggaaaaagtctctcaacattaggtctcacttggaagccagttctgaccagtggaagcgtctgcacctttctcttcaggaacttctggtatggctccagctgaaagatgatgagttaagccggcaggcacccattggaggagactttccagcggtgcagaagcagaatgatgtacacagggccttcaagagggaattgaaaacgaaagaacctgtaatcatgagtactcttgagactgtacgaatatttctgacagagcagcctttagaaggactagagaaactctaccaggagcccagagagctgcctcctgaagagagagcccagaatgtcacacggctcctacgaaagcaagctgaggaggtcaacactcagtgggaaaaactgaacgtgcactctgcagactggcagagaaaaatagacgaggccctcgaaagactccaggagcttcaggaagcaacagatgagctggatctcaaactacgtcaggcagaggtgatcaaaggatcctggcagcctgtgggtgacctcctcattgactctctccaagatcacctcgaaaaagtcaaggcgcttcgaggagaaattacacctctgaaagagaatgtcagctacgtcaatgaccttgctcgccaactcactacgttgggcattcagctgtcaccatataacctcaacactctggaagacctgaacaccagatggaagcttctgcaggtggccattgaggaccgcatcaggcagctgcatgaagcgcacagggactttggaccagcctcccagcacttcctttccacttctgtccagggtccctgggagagagccatctcaccaaacaaagtgccctactatatcaaccacgagacccaaacaacttgctgggaccatcccaaaatgacagagctctaccagtctttagctgacctgaataatgtcaggttctcagcttacaggactgccatgaaactccgaagactgcagaaggccctttgcttggatctcttgagcctatcggctgcatgcgatgccttggaccagcacaacctcaagcaaaatgaccagcccatggatatcctgcaggtcattaactgtctgaccactatttatgatcgcctagagcaagagcacaacaatctggtcaacgtccctctctgcgtggatatgtgtctcaattggctgctgaatgtttatgacacgggacgaacggggaggatccgggtcctgtcttttaaaactggcatcatttctctgtgtaaagcccatttggaagacaagtacagatacctcttcaagcaagtggcaagttcgacaggattttgtgaccagcgcaggctgggcctcctcctgcatgactctatccagatcccaagacagttgggtgaagtcgcatcgttcgggggcagtaacattgagccgagtgtcaggagctgcttccagtttgctaataataagcctgagatcgaagcggccctcttcctagactggatgcgcctggagccccagtccatggtgtggctgcctgtcctgcaccgagtggctgccgcggaaactgccaagcaccaggccaagtgcaacatctgcaaggagtgtcccatcatcggattcaggtacaggagtctaaagcactttaattatgacatctggcaaagttgctttttttctggtcgagttgcaaaaggccataaaatgcactatcccatggtggaatactgcactccgactacatcgggagaagatgtccgtgactttgccaaggtactaaaaaacaaatttcgaaccaaaaggtattttgcgaagcatccccgaatgggctacctgccagtgcagactgtcttagagggggacaacatggaaactcctgtcactctgatcaacttctggccggtagattctgcgcctgcctcgtcccctcagctttcacacgatgatactcattcacgcattgagcattatgctagcaggctaaaaaaaatggaaaacagcaatggatcttatctaaatgatagcatctctcctaatgagagcatagatgatgaacatttgttaatccagcattactggcgaagtttgaaccaggaatcccccctgagccagcctcgtagtcctgcccagatcttgatttccttagagagtgaggaaagaggggagctagagagaatcctagcagatcttgaggggagaaacagaaatctgcaagcagagtatgatcgtctaaagcagcagcatgaacacaaaggcctgtccccactgccatcccctcctgaaatgatgcctacttctccccaaagtccccgggatgctgagctcatcgctgaggccaagctgctgcgtcaacacaaaggccgcctggaagccaggatgcaaatccttgaagaccataacaaacaactggaatcccagttacacaggctcaggcagctgctggagcaaccccaggcagaggccaaggtgaatggtacaacggtgtcttctccttctacctctcttcagaggtcagatagcagtcagcctatgctgctccgggtggtcggcagtcagacttcagaatccatgggcgaggaagacctgctcagccctccccaggacacaagcacagggttagaggaagtgatggagcagctcaaccactccttccctagttccagaggaagaaatacccctgggaagccaatgagagaggacacaatgtag

SEQ ID NO: 49 - protein, 3680 aa, Canis lupus
MLWWEEVEDCYEREDVQKKTFTKWVNAQFSKFGKQHIENLFSDLQDGRRLLDLLEGLTGQKLPKEKGSTRVHALNNVNKALRVLQKNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIAKYQLGIEKLLDPEDVATTYPDKKSILMYITSLFQVLPQQVSIEAIQEVEMLPRPSQVTREEHFQIHHQMHYSQQITVSLAQGYERAPSFPKPRFKSYAYTQAAYVTTSDPTRSPLPSQHLETPEDKSFGRSLTETEANLDSYQTALEEVLSWLLSAEDALQAQGEISNDVEEVKEQFHTHEGYMMDLTSHQGRVGNVLQLGSQLIGTGKLSEDEETEVQEQMNLLNSRWECLRVASMEKQSNLHKVLMDLQNQQLKELNDWLTKTEERTRKMEKEPLGPDIEDLKRQVQQHKVLQEDLEQEQVRVNSLTHMVVVVDESSGDHATAALEEQLKVLGDRWANICRWTEDRWVLLQDILLKWQRFTEEQCLFSAWLSEKEDAVNKIHTTGFKDQSEVLSNLQKLAVLKTDLEKKKQTMDKLCSLNQDLLSALKNTVVAHKMEAWLDNFAQRWDNLVQKLEKSSAQISQAVTTTQPSLTQTTVMETVTMVTTREHILVKHAQEELPPPPPQKKRQIIVDSEIRKRLDVDITELHSWITRSEAVLQSPEFAIYRKEGNFSDLKEKVNAIEREKAEKFRKLQDASRSAQALVEQMVNEGVNADSIKQASEQLNSRWIEFCQLLSERLNWLEYQNNIITFYNQLQQLEQMTTTAENWLKTQPTTTSEPTAIKSQLKICKDEINRLSALQPQIERLKIQSIALKEKGQGPMFLDADFVAFTNHFNQVFADVQAREKELQTIFDSLPPMRYQETMSTILTWIQQSETKLSIPQVTVTEYDIMEQRLGELQALQSSLQEQQNGLNYLSTTVKEMSKKAPLSDISRKYQSEFEEIEGRWKKLSSQLVEHCQKLEEQMAKLRKIQNHIKTLKKWITEVDVFLKEEWPALGDSEILKRQLKQCRLLVNDIQTIQPSLNSVNEGAQKMKNEAEPEFAGRLETELRELNTQWDYMCRQVYARKEALKGGLDKTVSLQKDLSEMHEWMTQAEEEYLERDFEYKTPDELQTAVEEMKRAKEEAQQKEAKVKLLTESVNSVIAQAPPAAQEALKKELDTLTTNYQWLCTRLNGKCKTLEEVWACWHELLSYLEKANKWLSEVEVKLKTTENISGGAEEIAEVLDSLENLMQHSEDNPNQIRILAQTLTDGGVMDELINEELETFNSRWRELHEEAVRRQKLLEQSIQSAQEIEKSLHLIQESLSSIDKQLAAYIADKVDAAQMPQEAQKIQSDLTSHEISLEEMKKHNQGKETAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNALTEWLAATDMELTKRSAVEGMPSNLDSEVAWGKATQKEIEKQKVHLKSVTEVGEALKTVLGKKEMLVEDKLSLLNSNWIAVTSRAEEWLNLLLEYQKHMETFDQNVDYITNWIIQADALLDESEKKKPQQKEDILKRLKAEMNDIRPKVDSTRDQAANLMANRGDHCRKVVEPKISELNHRFAAISHRIKTGKASIPLKELEQFNSDIQKLLEPLEAEIQQGVNLKEEDFNKDMSEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVHEESVVAMTEDMPLEISYVPSTYLTEITHVSQALSEVEELLNAPDLCAQDFEDLFKQEESLKNIKDSLQQISGRIDIIHNKKTAALHSATPAERAKLQEALSRLDFQWERVNNMYKDRQGRFDRSVEKWRRFHYDMKILNQWLTEAEQFLKKTQIPENWEHAKYKWYLKELQDGIGQRQSVVRVLNATGEEIIQQSSKTDASILQEKLGSLNLRWQEVCKQLAERKKRLEEQKNILSEFQRDVNEFVLWLEEADNVANIPLEPGNEQQLKEKLEQVKLLAEELPLRQGILKQLNETGGTVLVSAPLSPEEQDKLENKLKQTNLQWIKVSRNLPEKQEEIEAHVKDLGQLEEQLNHLLLWLSPIRNQLEIYNQPNQTGPFDIKEIEVAVQAKQPDVEGILSKGQHLYKEKPATQPAKRKLEDLSSDWKVVTQLLQELRAKQPGPAPGLTTVRAPPSQTVTLVTQPAVTKETAISKLEMPSSLLLEVPALADFNRAWTELTDWLSLLDRVIKSQRVMVGDLEDINEMIIKQKATLQDLEQRRPQLEELITAAQNLKNKTSNQEARTIITDRIERIQSQWDEVQEHLQNRRLQLTEMLKDSTQWLEAKEEAEQVLGQARAKLESWKEAPYTVDAIQKKITETKQLAKDLRQWQINVDVANDLALKLLRDYSADDTRKVHMITENINASWASIHKRLSEREAALEETHRLLQQFPLDLEKFLAWLTEAETTANVLQDATHKERLLEDSKGVRELMKQWQDLQGEIEAHTDIYHNLDENGQKVLRSLEGSDDAALLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTQWEKLNVHSADWQRKIDEALERLQELQEATDELDLKLRQAEVIKGSWQPVGDLLIDSLQDHLEKVKALRGEITPLKENVSYVNDLARQLTTLGIQLSPYNLNTLEDLNTRWKLLQVAIEDRIRQLHEAHRDFGPASQHFLSTSVQGPWERAISPNKVPYYINHETQTTCWDHPKMTELYQSLADLNNVRFSAYRTAMKLRRLQKAICLDLLSLSAACDALDQHNLKQNDQPNDILQVINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLKKMENSNGSYLNDSISPNESIDDEHLLIQHYWRSLNQESPLSQPRSPAQILISLESEERGELERILADLEGRNRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSESMGEEDLLSPPQDTSTGLEEVMEQLNHSFPSSRGRNTPGKPMREDTM

SEQ ID NO: 50 - protein, 251 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVE

SEQ ID NO: 51 - protein, 2860 aa, Homo sapiens
MLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSGLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKL

SEQ ID NO: 52 - protein, 296 aa, Homo sapiens
RRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 53 - protein, 277 aa, Homo sapiens
PVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 54 - protein, 15 aa, Homo sapiens
LLNSRWECLRVASME

SEQ ID NO: 55 - protein, 15 aa, Homo sapiens
QRLTEEQCLFSAWLS

SEQ ID NO: 56 - protein, 15 aa, Homo sapiens
WLDNFARCWDNLVQK

SEQ ID NO: 57 - protein, 8 aa, Homo sapiens
CLKLSRKM

SEQ ID NO: 58 - protein, 110 aa, Homo sapiens
EISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDR

SEQ ID NO: 59 - protein, 21 aa, Homo sapiens
PSTYLTEITHVSQALLEVEQL

SEQ ID NO: 60 - protein, 309 aa, Homo sapiens
SIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFELRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLE

SEQ ID NO: 61 - protein, 331 aa, Homo sapiens
SEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQ

SEQ ID NO: 62 - protein, 4 aa, Homo sapiens
GGSG

SEQ ID NO: 63 - protein, 4 aa, Homo sapiens
GGGS

SEQ ID NO: 64 - protein, 5 aa, Homo sapiens
GGGGS

SEQ ID NO: 65 - protein, 4 aa, Homo sapiens
GSAT

SEQ ID NO: 66 - protein, 96 aa, Mus musculus
LLLQRRRVTVRKADAGGLGISIKGGRENKMPILISKIFKGLAADQTEALFVGDAILSVNGEDLSSATHDEAVQALKKTGKEVVLEVKYMKEVSPYF

SEQ ID NO: 67 - protein, 96 aa, Homo sapiens
LLLQRRRVTVRKADAGGLGISIKGGRENKMPILISKIFKGLAADQTEALFVGDAILSVNGEDLSSATHDEAVQVLKKTGKEVVLEVKYMKDVSPYF

(19) United States
(12) Patent Application Publication
(10) Pub. No.: US 2024/0148904 A1
(43) Pub. Date: May 9, 2024

(54) DYSTROPHIN CONSTRUCTS CONTAINING R11-R12

(71) Applicant: THE CURATORS OF THE UNIVERSITY OF MISSOURI, Columbia, MO (US)

(72) Inventors: Dongsheng Duan, Columbia, MO (US); Yi Lai, Columbia, MO (US); Junling Zhao, Columbia, MO (US); Yongping Yue, Columbia, MO (US)

(21) Appl. No.: 18/511,568

(22) Filed: Nov. 16, 2023

Related U.S. Application Data
(63) Continuation of application No. 17/555,233, filed on Dec. 17, 2021.
Application No. 17/555,233 is a continuation of application No. 16/311,236, filed on Dec. 19, 2018, now Pat. No. 11,202,840.
(60) Provisional application No. 62/367,559, filed on Jul. 27, 2016.
(60) Provisional application No. 62/357,865, filed on Jul. 1, 2016.
(60) Provisional application No. 62/352,927, filed on Jun. 21, 2016.

(51) Int. Cl. A61K 48/00; A61K 9/00; A61K 35/28; A61P 25/14; C07K 14/47; C12N 5/0775; C12N 15/62; C12N 15/85; C12N 15/86

(52) U.S. Cl. CPC A61K 48/005; A61K 9/0019; A61K 35/28; A61K 48/00; A61P 25/14; C07K 14/4716; C12N 5/0663; C12N 15/62; C12N 15/85; C12N 15/86; A61K 38/00; C07K 2319/00

(57) ABSTRACT

Synthetic nucleic acids encoding mini and microdystrophin genes comprising the membrane binding motifs or domains of the R10-R11-R12 region are provided. Also provided are vectors, host cells, and related methods of using the same to treat a subject suffering from Duchenne muscular dystrophy (DMD), Becker muscular dystrophy (BMD) or X-linked dilated cardiomyopathy (XLDC), or for ameliorating one or more adverse effects of DMD, BMD, or XLDC. Also provided are a fusion protein comprising a nNOS binding domain of dystrophin R16-R17 that is operably linked to a syntrophin PDZ domain and synthetic nucleic acids comprising the same that can be used to treat subjects with diseases characterized by loss of sarcolemmal neuronal nitric oxide synthase (nNOS) activity.

### CROSS REFERENCE TO RELATED APPLICATIONS

This application is a Continuation of U.S. application Ser. No. 16/311,236, filed on Dec. 19, 2018, issued as U.S. Pat. No. 11,202,840, which is a U.S. National Phase application of International Patent Application PCT/US2017/038418, filed on Jun. 21, 2017, which claims the benefit of U.S. Provisional Patent Application No. 62/367,559, filed on Jul. 27, 2016; U.S. Provisional Patent Application No. 62/357,865, filed on Jul. 1, 2016; and U.S. Provisional Patent Application No. 62/352,927, filed on Jun. 21, 2016, each of which is incorporated herein by reference in its entirety.

### STATEMENT REGARDING FEDERALLY-SPONSORED RESEARCH AND DEVELOPMENT

This invention was made with government support under AR067985, AR049419, and NS090634 awarded by the National Institutes of Health. The government has certain rights in the invention.

### INCORPORATION OF SEQUENCE LISTING

A sequence listing in the file named “corrected_17UMC006_CON_SEQLIST_232545.xml”, which is 190,820 bytes (measured in MS-Windows®), contains 67 sequences, and was created on Jan. 23, 2024, is provided herewith via the USPTO's Patent Center, and is incorporated herein by reference in its entirety.

### BACKGROUND

Dystrophin is an essential cytoskeletal protein in the muscle. It constitutes a primary linkage between the extracellular matrix (ECM) and the actin cytoskeleton (1, 2). In muscle cells, dystrophin plays an important role in maintaining membrane integrity and preventing membrane rupture. Loss of dystrophin, as seen in Duchenne muscular dystrophy (DMD) (3), leads to sarcolemmal leakage, myofiber degeneration and necrosis. Full-length dystrophin is a large rod-shaped protein. It contains four functional domains including N-terminus (NT), the mid-rod domain, the cysteine-rich (CR) domain and C-terminus (CT). The mid-rod domain consists of 24 spectrin-like repeats. Four hinges (H) are interspersed in the mid-rod domain (4). Dystrophin NT and spectrin-like repeats R11-17 bind to cytoskeletal filamentous actin (5, 6). The CR domain anchors dystrophin to the muscle membrane via interaction with the transmembrane protein β-dystroglycan (7-9). β-dystroglycan further connects with basal lamina proteins to complete the axis from the ECM to the cytoskeleton (10). This mechanical linkage protects the muscle membrane from contraction-induced damages. In this well-established model, the dystrophin CR domain is solely responsible for dystrophin membrane binding (FIG. 1).

Despite compelling evidence suggesting that the CR domain mediates dystrophin-sarcolemma interaction, case reports from some rare-occurring patients suggest that dystrophin can bind to the sarcolemma through CR domain-independent mechanisms. In these patients, biochemical and genetic analyses confirmed a complete deletion of the CR domain. Yet, immunostaining showed clear sarcolemmal localization of the truncated dystrophin protein (FIG. 2B) (11-13).

### SUMMARY

Synthetic nucleic acid molecules encoding a synthetic mini-dystrophin gene or micro-dystrophin gene encoding a synthetic, non-full length dystrophin protein comprising: (i) an N-terminal (NT) domain of the dystrophin protein or a modified N-terminal domain of the dystrophin protein; (ii) at least two membrane binding motifs (MBM) independently selected from the group consisting of an MBM of an R1-R2-R3 membrane binding domain (MBD), an MBM of a CR membrane binding domain, and an MBM of a CT membrane binding domain; (iii) an MBM of an R10-R11-R12 MBD; and (iv) an nNOS binding domain of R16-R17; wherein the domains and the MBM are arranged from N to C terminus in the order in which they occur in a wild-type dystrophin protein and are operably linked are provided. Synthetic nucleic acid molecules encoding a synthetic mini-dystrophin gene or micro-dystrophin gene encoding a synthetic, non-full length dystrophin protein comprising: (i) an N-terminal (NT) domain of the dystrophin protein or a modified N-terminal domain of the dystrophin protein; (ii) at least two membrane binding motifs (MBM) independently selected from the group consisting of an MBM of an R1-R2-R3 membrane binding domain (MBD), an MBM of a CR membrane binding domain, and an MBM of a CT membrane binding domain; (iii) an MBM of an R10-R11-R12 MBD; and (iv) an nNOS binding domain of R16-R17 that is operably linked to a syntrophin PDZ domain; wherein the dystrophin domains and the MBM are arranged from N to C terminus in the order in which they occur in a wild-type dystrophin protein and are operably linked are also provided. A synthetic nucleic acid molecule comprising a sequence encoding a fusion protein comprising a nNOS binding domain of dystrophin R16-R17 that is operably linked to a syntrophin PDZ domain are also provided. In certain embodiments, the nNOS binding domain of dystrophin R16-R17 is operably linked to a syntrophin PDZ domain with a hinge region in the fusion protein. In certain embodiments, the nNOS binding domain of dystrophin R16-R17 is operably linked to a syntrophin PDZ domain with a hinge region selected from the group consisting of a synthetic hinge, a semi-synthetic hinge, dystrophin H1, dystrophin H2, dystrophin H3, dystrophin H4, and variants thereof. In certain embodiments, the MBM of R1-R2-R3 comprises at least one S-palmitoylation site peptide selected from the group consisting of SEQ ID NO: 54, SEQ ID NO: 55, and SEQ ID NO:56. In certain embodiments, the R3 repeat or R2-R3 repeats are absent from the non-full length dystrophin protein. In certain embodiments, the R1, R2, R3, R1 and R2, R2 and R3, or R1, R2, and R3 repeats are present in the non-full length dystrophin protein. In certain embodiments, the MBM of R10-R11-R12 comprises an S-palmitoylation site peptide of SEQ ID NO:57. In certain embodiments, the R10 repeat, the R11 repeat, the R12 repeat, the R10-R11 repeats, the R11-R12, or the R10 and R12 repeats are present in the non-full length dystrophin protein. In certain embodiments, the R17 domain is present in the non-full length dystrophin protein. In certain embodiments, the n-terminal alpha helix of the R16 domain (SEQ ID NO:59) or a portion thereof is absent from the non-full length dystrophin protein. In certain embodiments, alpha-helix 2 and alpha-helix 3 of the R16 domain is present and alpha-helix 1, alpha-helix 2, and alpha-helix 3 of the R17 domain is present in the non-full length dystrophin protein. In certain embodiments, alpha-helix 2 and alpha-helix 3 of the R16 domain is present and alpha-helix 1, alpha-helix 2, and alpha-helix 3 of the R17 domain is present in the non-full length dystrophin protein. In certain embodiments, the N-terminal helix one of the R16 domain is substituted with the MBM of the R1-R2-R3 MBD or with the MBM of the R10-R11-R12 MBD. In certain embodiments, the R16 domain and the R17 domain are present in the non-full length dystrophin protein. In certain embodiments, the MBM of the CR membrane binding domain is absent, wherein the CR membrane binding domain is absent, or wherein the CR domain is absent from the non-full length dystrophin protein. In certain embodiments, the MBM of the CT MBD comprises residues 3422 to 3535 of SEQ ID NO: 1. In certain embodiments, the MBM of the CT MBD comprises residues 3501 to 3685 of SEQ ID NO:1. In certain embodiments, at least one domain and at least one MBM are operably linked with a hinge region selected from the group consisting of a synthetic hinge, a semi-synthetic hinge, dystrophin H1, dystrophin H2, dystrophin H3, dystrophin H4, and variants thereof. In certain embodiments, the dystrophin H1 hinge or a variant thereof operably links the C-terminus of the NT domain to the N-terminus of an MBM or domain containing an MBM, wherein the dystrophin H2 hinge or a variant thereof operably links the C-terminus of a MBM or domain containing an MBM to the N-terminus of another MBM or domain containing another MBM, wherein the dystrophin H3 hinge or a variant thereof operably links the C-terminus of an MBM or domain containing an MBM to the N-terminus of another MBM or domain containing another MBM, wherein the dystrophin H4 hinge or a variant thereof operably links the C-terminus of an MBM to the N-terminus of the CR MBM or the CR domain, or any combination thereof. In certain embodiments, the dystrophin H4 hinge or a variant thereof operably links the C-terminus of an MBM to the N-terminus of the CR MBM or the CR domain. In certain embodiments of any of the aforementioned synthetic nucleic acid molecules, the mini- or micro-dystrophin gene is between 5 kb to about 8 kb in length or less than 5 kb in length, respectively. In certain embodiments of any of the aforementioned synthetic nucleic acid molecules, the mini- or micro-dystrophin gene is operably linked to a heterologous promoter, a heterologous 5′ untranslated region (UTR), a heterologous 3′ UTR, a heterologous polyadenylation site, or any combination thereof. In certain embodiments of any of the aforementioned synthetic nucleic acid molecules, the molecule is integrated within an endogenous dystrophin gene locus in an X-chromosome.

Lentiviral vectors comprising any of the aforementioned synthetic nucleic acid molecules, wherein the nucleic acid molecule is operably linked to an expression cassette, 5′ and 3′ long terminal repeats (LTR), and a psi sequence in the lentiviral vector are also provided.

Single recombinant adeno-associated virus (AAV) vector comprising any of the aforementioned synthetic nucleic acid molecules, wherein said nucleic acid molecule is operably linked to an expression cassette and viral inverted terminal repeats (ITRs) in the AAV are also provided.

Dual recombinant AAV vector system, comprising two AAV vectors, wherein one of the two AAV vectors comprises a part of the nucleic acid molecule of any one of the aforementioned synthetic nucleic acid molecules, and the other vector comprises the remaining part of said nucleic acid molecule, wherein the two vectors further comprise sequences that permit recombination with each other to produce said nucleic acid in full length, and wherein the nucleic acid in full length is operably linked to an expression cassette and viral ITRs.

Composition comprising any one of the aforementioned synthetic nucleic acid molecules or vectors and a pharmaceutically acceptable carrier are also provided. In certain embodiments, the synthetic nucleic acid molecule is operably linked to an expression cassette, 5′ and 3′ long terminal repeats (LTR), and a psi sequence in a lentiviral vector. In certain embodiments, the nucleic acid molecule is operably linked to an expression cassette and viral inverted terminal repeats (ITRs) in an AAV. In certain embodiments, the composition comprises the aforementioned dual recombinant AAV vector system.

Isolated host cells comprising any one of the aforementioned synthetic nucleic acid molecules or vectors are also provided. In certain embodiments, the nucleic acid molecule is integrated within an endogenous dystrophin gene locus in a chromosome of the host cell. In certain embodiments, the nucleic acid molecule is operably linked to an expression cassette, 5′ and 3′ long terminal repeats (LTR), and a psi element in a lentiviral vector. In certain embodiments, the nucleic acid molecule is operably linked to an expression cassette and ITRs in an AAV. In certain embodiments, the host cell is a myogenic stem cell.

Methods for the treating or ameliorating one or more adverse effects of Duchenne muscular dystrophy (DMD), Becker muscular dystrophy (BMD), X-linked dilated cardiomyopathy (XLDC), age-related muscle atrophy, cancer cachexia, or other neuromuscular disorders characterized by loss of sarcolemmal neuronal nitric oxide synthase (nNOS) activity in a subject in need thereof comprising the step of administering to the subject a therapeutically effective amount of: (i) any one of the aforementioned synthetic nucleic acid molecules; (ii) the aforementioned lentiviral vectors; (iii) the aforementioned AAV vectors; (iv) any one of the aforementioned compositions; or (iv) any one of the aforementioned host cells to a subject in need thereof. In certain embodiments, the administration is by injection into muscle, systemic delivery, or local delivery. In certain embodiments, the host cell is a stem cell or myogenic stem cell. In certain embodiments, the host cell is derived from an autologous cell of the subject. In certain aforementioned methods, a defective endogenous dystrophin gene of the host cell or a defective portion thereof is edited to provide the synthetic nucleic acid molecule within the host cell's X-chromosome.

Use of (i) any one of the aforementioned synthetic nucleic acid molecules; (ii) the aforementioned lentiviral vectors; (iii) the aforementioned AAV vectors; (iv) any one of the aforementioned compositions; or (iv) any one of the aforementioned host cells for making a composition for administration to a subject suffering from Duchenne muscular dystrophy (DMD), Becker muscular dystrophy (BMD), X-linked dilated cardiomyopathy (XLDC) age-related muscle atrophy, cancer cachexia, or other neuromuscular disorders characterized by loss of sarcolemmal neuronal nitric oxide synthase (nNOS) activity is also provided.

Use of (i) any one of the aforementioned synthetic nucleic acid molecules; (ii) the aforementioned lentiviral vectors; (iii) the aforementioned AAV vectors; (iv) any one of the aforementioned compositions; or (iv) any one of the aforementioned host cells for treating a subject suffering from Duchenne muscular dystrophy (DMD), Becker muscular dystrophy (BMD) or X-linked dilated cardiomyopathy (XLDC), or for ameliorating one or more adverse effects of DMD, BMD, XLDC, age-related muscle atrophy, cancer cachexia, or other neuromuscular disorders characterized by loss of sarcolemmal neuronal nitric oxide synthase (nNOS) activity is also provided.

### BRIEF DESCRIPTION OF THE DRAWINGS

FIG. 1. The classic model of dystrophin-sarcolemma interaction. Numerous studies suggest that dystrophin binds to the sarcolemma via its CR domain (1-8). See Supplementary References provided herein for full citation.

FIG. 2A,B,C,D. Evidence of dystrophin sarcolemmal binding in the absence of the CR domain. A, Cartoon illustration of the structure of full-length dystrophin. B, Cartoon illustration of CR-deleted dystrophins that were found at the sarcolemma in patients (9-11). C, Cartoon illustration of synthetic CR-deleted dystrophin fragments that showed sarcolemmal localization in mdx mice (12-17). D, Cartoon illustration dystrophin membrane binding domains identified by in vitro interaction assays (18-23). Related references are marked next to the cartoon illustrations and the full citation is available in Supplementary References provided herein. Filled shapes: domains present; open shapes: domains absent.

FIG. 3. Dystrophin R1-3, CR and CT bind to the sarcolemma in the heart. Indicated GFP fusion dystrophin subdomains were delivered to the mdx heart by systemic AAV injection. Uninjected BL10 and mdx hearts were used as negative controls. Subdomain H4-CR and CT showed membrane localization. Subdomain R1-3 was found in the intercalated disk and cytosol. Remaining subdomains were only seen in the cytosol. Scale bar: 50 μm.

FIG. 4. The CR domain in ΔR4-R23/ΔCT is replaced with the CT domain. The membrane binding is marked by underlining.

FIG. 5. Cartoon illustration of ten GFP-fused dystrophin subdomains used in the study. The full-length human dystrophin molecule is split into ten subdomains. The numerical number range above each cartoon illustration refers to amino acid sequence numbering in the full-length human dystrophin protein. The predicted molecular weight of each fusion protein is marked. The YL numbers refer to the construct name in the Duan/Lai laboratory.

FIG. 6. Dystrophin R1-3, R10-12, CR and CT are independent membrane-binding domains. Full-length human dystrophin was split into ten subdomains and each subdomain fused with a GFP tag. The fusion proteins were individually expressed in mdx muscle by AAV gene transfer. Representative GFP photomicrographs of each indicated dystrophin subdomain are shown. Dystrophin R1-3, H4-CR and CT were exclusively localized at the sarcolemma. R10-12 was found at the sarcolemma and in the cytosol. NT-H1, R4-6, R7-9, R13-15, R16-19 and R20-24 were exclusively localized in the cytosol. Scale bar: 50 μm.

FIG. 7A,B. Microsomal western blot suggests the association of R1-3, R10-12, CR and CT with the sarcolemma. A. Whole muscle lysate western blots revealing AAV-mediated expression of GFP-fused dystrophin subdomains in mdx muscle. B. Detection of dystrophin R1-3, R10-12 CR and CT in the membrane fraction by microsomal western blots. GAPDH marks the cytosolic fraction. C, cytosolic fraction; M, membrane fraction.

FIG. 8. Dystrophin R1-3, R10-12, CR and CT bind to the sarcolemma in canine muscle. Indicated GFP fusion dystrophin subdomains were expressed in dystrophic dog muscle by AAV gene transfer. Representative GFP photomicrographs show the membrane binding of R1-3, R10-12, CR and CT and cytosolic localization of R7-9 and R20-24. R10-12 is also seen in the cytosol. Scale bar: 50 μm.

FIG. 9. The hypothetical mechanism of CT-mediated DGC restoration. Left side cartoon illustrates the CR domain mediated DGC restoration. Right side cartoon illustrates the hypothetical mechanism of CT-mediated DGC restoration. Specifically, direct membrane binding of the CT domain restores syntrophin and dystrobrevin to the sarcolemma (24, 25). Membrane-localized syntrophin and dystrobrevin then recruit sarcoglycans and dystroglycan to the sarcolemma (26-29). DG, dystroglycan; SG, sarcoglycans; Dbr, dystrobrevin; Syn, syntrophin.

FIG. 10. Dystrophin CT restores the DGC at the sarcolemma. Representative serial section photomicrographs of GFP and immunostaining for β-dystroglycan, β-sarcoglycan, dystrobrevin and syntrophin in mdx muscle expressing the indicated GFP-dystrophin subdomain fusion proteins. Asterisk, the GFP-positive myofiber in serial sections; triangle, the GFP-negative revertant fiber in serial sections. GFP signals co-localize with DGC components in myofibers transduced by the H4-CR and CT but not R1-3 and R10-12 subdomain AAV vectors. Scale bar: 50 μm.

FIG. 11A,B. A new model of dystrophin-sarcolemma interaction. A. In muscle, dystrophin binds to the sarcolemma through four independent membrane-binding subdomains; B. In the heart, dystrophin binds to the sarcolemma through three independent membrane-binding domains. These subdomains are marked by thick red lines.

FIG. 12. Position of cysteine residues in R1-3, R10-12 and CT is conserved between human and mouse dystrophin.

FIG. 13. Identification of potential palmitoylated site peptides in R1-3 and R10-12 by CSS-Palm 2.0 program. The predicted palmitoylation sites are the sole cysteine residues in the sequences. From top to bottom, the palmitoylation site peptide sequences are LLNSRWECLRVASME (SEQ ID NO:54), QRLTEEQCLFSAWLS (SEQ ID NO:55), WLDNFARCWDNLVQK (SEQ ID NO:56), and CLKLSRKM (SEQ ID NO:57).

FIG. 14. Shows that cysteine mutations (C to S mutation) disrupt membrane binding of R1-3, R10-12 and CT. In R10-12, cysteine mutations also causes protein aggregates. Images shown as the GFP signal. Cys mut: cysteine mutant.

FIG. 15. Both CR and CT domain are associated with the DGC components. The DGC staining was performed in the sections expressed with H4-CR.GFP, CT.GFP, R1-3.GFP and R10-12.GFP. Both CR and CT domain are associated with the DGC at the muscle membrane, while R1-3 and R10-12 are not co-localized with the DGC at the sarcolemma. White asterisk: the GFP-positive fiber; arrow: the revertant fiber.

FIG. 16A,B. The constructs for detecting whether individual repeats from R1-3 and R10-12 maintain the membrane-binding ability. A. Cytosolic distribution of R16/17.GFP in the muscle of mdx and ΔH2-R19 mini-dystrophin transgenic mice. B. The construct design for the example.

FIG. 17. Comparison of the functional roles of R1-3 and R10-12. R1-3 is replaced with R10-12 in micro- and mini-dystrophin. The membrane binding is marked by underlining.

FIG. 18. The constructs with partial deletion of the CT domain for detecting the membrane-binding motif in CT. The CT domain tested here is from amino acid 3422 to 3685. The Roman numerals indicate the partial CT domains with different boundaries. The boundary of the constructs is labeled by the number of amino acid. These partial CT domains will be fused to GFP and expressed by AAV gene transfer.

FIG. 19. The new micro- and mini-dystrophins. The original ΔR4-R23/ΔCT only contains two MBDs. We will generate ΔR4-R23 micro-dystrophin with three MBDs. The mini-dystrophin ΔH2-R19 contains three MBDs. We will add R10-12 to ΔH2-R19 minigene to make ΔH2-R9/ΔR13-R19 new minigene with four MBDs. The MBDs are marked by underlining.

FIG. 20. Membrane binding of the rMBD, R1-3 was disrupted by cysteine mutations, or by replacement with R4-6 in micro- and mini-dystrophin. The membrane binding is marked by red underline.

FIG. 21. Currently available micro-dystrophins used in a clinical trial (μDys-1; Mendell, J. R. et al. N. Engl. J. Med. 363, 1429-1437 (2010)) or in large animal models (μDys-2; Wang, Z. et al., Mol Ther 20, 1501-1507 (2012) and μDys-3; Yue, Y. et al. Hum. Mol. Genet. 24, 5880-5890 (2015)). They contain a partial or complete rMBD and a complete cMBD, indicated by underlining.

FIG. 22. Schematic diagram of dystrophin and its membrane binding domains.

FIG. 23. Methodology for evaluating synthetic mini-dystrophin gene or micro-dystrophin gene constructs.

FIG. 24. The construct design of AAV.R16/17.Syn.GFP.Pal. To induce the expression of R16/17.Syn PDZ.GFP.Pal in the muscle, we will engineer an AAV construct. Syntrophin PDZ domain is fused to the C-terminus of dystrophin R16/17. We add green fluorescent protein (GFP) as the tag to help detection of R16/17.Syn fusion protein. Pal is the signal for membrane targeting. The expression of R16/17.Syn.GFP.Pal is driven by CMV promoter and SV40 polyA. ITR (inverted terminal repeat) is the sequence for AAV virus production.

FIG. 25. Sarcolemmal nNOS was recovered successfully in a mdx mouse with the use of the R16/17-syntrophin PDZ fusion protein. Illustrated above are the expression levels of nNOS in different mice controls.

FIG. 26. Schematic diagram of dystrophin domains that do or do not exhibit membrane binding.

FIG. 27A,B. A. Dystrophin functional domains and dystrophin nNOS-binding domain. Dystrophin is composed of four functional domains: NT: N-terminus; the mid rod domain; CR: cysteine-rich domain; and CT: C-terminus. The mid-rod domain contains 24 spectrin-like repeats and four hinge (H) regions. Dystrophin spectrin-like repeats 16 and 17 (R16/17) are identified as the nNOS-binding domain. B. Sarcolemmal localization of nNOS is dependent on interactions with dystrophin R16/17 and syntrophin. Both dystrophin R16/17 and Syntrophin (Syn) bind to nNOS. The interaction of nNOS with dystrophin R16/17 and syntrophin anchors nNOS to the sarcolemma. Syn: Syntrophin; DG: Dystroglycan.

### DETAILED DESCRIPTION

The present disclosure identifies a novel series of dystrophin minigenes and microgenes that are small enough to be packaged into AAV or lentiviral vectors, and yet retain functions of a full-length, wild type dystrophin gene, including, but not limited to, the membrane binding functions and signal functions (such as sarcolemmal nNOS-related functions), needed for protecting muscle from dystrophic injury. The present disclosure recognizes that the inclusion of membrane binding motifs and/or the entire membrane binding domains contained in the spectrin repeats R10-R11-R12 of the mid-rod domain of a dystrophin protein in a synthetic mini/micro-dystrophin gene provide useful membrane binding functions. Mini or micro-dystrophin genes retaining the membrane binding motifs or membrane binding domains of the R10-R11-R12 can exhibit improved membrane binding and biological activity in comparison to mini or micro-dystrophin genes that lack the membrane binding motifs or membrane binding domains of the R10-R11-R12.

By “domain” is meant a portion of a protein structure. For example, the “N-terminal domain” or “NT” of a human dystrophin protein, as referred to herein, includes amino acid residues from approximately 1 to approximately 252, particularly, from amino acid residues methionine 1 to glutamate 252 of SEQ ID NO: 1, more particularly, amino acid sequence encoded by a nucleotide sequence as set forth in SEQ ID NO: 17. Similarly, the “mid-rod domain” or “rod domain” of a dystrophin protein, as referred to herein, includes amino acid residues approximately from 253 to approximately 3112 of SEQ ID NO: 1, particularly, from amino acid residues methionine 253 to leucine 3112 as set forth in SEQ ID NO: 1; the “cysteine-rich domain” or “CR” of a dystrophin protein, as referred to herein, includes amino acid residues from approximately 3113 to approximately 3408 of SEQ ID NO: 1, particularly, from amino acid residues arginine 3113 to threonine 3048 as set forth in SEQ ID NO: 1, more particularly, amino acid sequence encoded by a nucleotide sequence as set forth in SEQ ID NO: 46 and the “C-terminal domain” or “CT” of a dystrophin protein, as referred to herein, includes amino acid residues from approximately 3409 to 3685 of SEQ ID NO: 1, particularly, from amino acid residues proline 3409 to methionine 3685 as set forth in SEQ ID NO: 47.

By “dystrophin microgene” or “micro-dystrophin gene” or “microgene” is meant a nucleic acid molecule that is 5 kb or less in length and encodes a modified or non-full-length dystrophin polypeptide (also referred to as micro-dystrophin in the present application) that retains the N-terminal domain, the cysteine-rich domain, two or more repeats of the mid-rod domain, and two or more hinges of the mid-rod domain of a full-length dystrophin protein. By “micro-dystrophin” is meant a modified or non-full-length dystrophin protein molecule that retains biological function of a full-length dystrophin protein and the coding sequence of which is 5 kb or less.

By “dystrophin minigene,” “mini-dystrophin gene” or “minigene” is meant a nucleic acid molecule that is more than 5 kb in length but less than the full-length of dystrophin coding sequence, between 5 kb to about 10 kb in length, about 5 kb to about 8 kb in length, or about 7 kb in length, and encodes a modified or non-full-length dystrophin polypeptide (also referred to as mini-dystrophin in the present application) that retains the N-terminal domain, the cysteine-rich domain, two or more repeats (also referred to by R and a number, e.g., R16 means repeat number 16) of the mid-rod domain, and two or more hinges of the mid-rod domain of a full-length dystrophin protein. By “mini-dystrophin” is meant a modified or non-full-length dystrophin protein molecule that retains the biological functions of a full-length dystrophin protein and the coding sequence of which is more than 5 kb in length but less than the full-length of dystrophin coding sequence.

By “biological functions” of a dystrophin protein is meant functions which include, but are not limited, at least one of providing a mechanical link between the sarcolemma, cytoskeleton or the extracellular matrix and/or providing a signaling function such as recruiting nNOS to the sarcolemma.

By “modified” in connection with dystrophin gene or dystrophin protein is meant a wild-type (or naturally-occurring) full-length dystrophin gene or dystrophin protein molecule is changed so that the modified dystrophin gene or dystrophin protein molecule does not include the full-length coding sequence of a dystrophin gene or the full-length amino acid sequence of a dystrophin protein, yet retain or substantially retain certain biological functions of a full-length gene or protein.

By “modified N-terminal domain” is meant an N-terminal domain that is different in structure and/or sequence from that of wild type or naturally occurred but retain the function of a wild type or naturally occurred N-terminus. By “modifications or variations” is meant any changes to a nucleic acid molecule or polypeptide, such as by mutation, that retains substantial function of the nucleic acid molecule or polypeptides and/or is substantially homologous with, or similar/identical to, the nucleic acid molecule or polypeptide.

In the classic model, dystrophin stabilizes the sarcolemma by interacting with a transmembrane protein β-dystroglycan and the F-actin cytoskeleton via its CR and NT domains, respectively. β-dystroglycan further connects with basal lamina proteins to complete the axis from the extracellular matrix (ECM) to intracellular cytoskeleton. However, this model completely ignores the direct interaction between dystrophin and membrane lipid bilayer, a major mechanism underlying spectrin-mediated membrane stabilization (Luna & Hitt, A. L. Science 258, 955-964 (1992); Le Rumeur et al. Biochim. Biophys. Acta 1804, 1713-1722 (2010); Sheetz, et al. Annu Rev Biophys Biomol Struct 35, 417-434 (2006)). Several lines of evidence suggest that dystrophin-lipid bilayer interaction can play a critical role for sarcolemma protection. First, in vitro studies suggest that the rod domain can contain putative lipid binding regions (LBRs) in R1-3 and R4-19 (Luna & Hitt, A. L. Science 258, 955-964 (1992); Le Rumeur et al. Biochim. Biophys. Acta 1804, 1713-1722 (2010); Sheetz, et al. Annu Rev Biophys Biomol Struct 35, 417-434 (2006)). Second, deletion of all putative rod domain LBRs abolishes the ability of dystrophin to protect muscle (Harper, S. Q. et al. Nat. Med. 8, 253-261 (2002)). Third, a series of in vitro studies demonstrated that binding of dystrophin LBRs to phospholipids considerably contributes to stiffness and stability of lipid monolayer (Sarkis, J. et al. FASEB J. 27, 359-367 (2013); Sarkis, J. et al. J. Biol. Chem. (2011)).

To better understand how dystrophin interacts with the sarcolemma in the absence of the CR domain, a comprehensive in vivo screening for alternative membrane binding domains (MBDs) in dystrophin was performed. The R1-3, R10-12 and CT domains were identified as new dystrophin MBDs in mouse muscle. We further confirmed that these MBDs are conserved in dog muscle. To determine whether these MBDs are functionally equivalent, we evaluated their ability to establish the dystrophin-associated glycoprotein complex (DGC) at the sarcolemma. Our results showed that only the CR domain and CT are capable of restoring the DGC. We also evaluated these newly discovered MBDs in the heart. We found that R1-3 and CT interact with the sarcolemma in cardiac muscle. Taken together, our studies suggest that dystrophin-sarcolemma interaction is much more complex than it has been perceived. Without seeking to be limited by theory, a new model to explain how dystrophin stabilizes the sarcolemma is proposed. In this model, dystrophin maintains sarcolemmal stability through two distinctive mechanisms: (i) dystrophin stabilizes the muscle membrane through the cytoskeleton (F-actin)-NT-CR-ECM axis; (ii) dystrophin strengthens the sarcolemma through the membrane association of its lipid binding regions LBRs. Both mechanisms involve the binding of dystrophin to the muscle membrane. Through the close association with the muscle membrane, dystrophin then tethers intracellular cytoskeleton to the sarcolemma, and stabilizes and strengthens the sarcolemma.

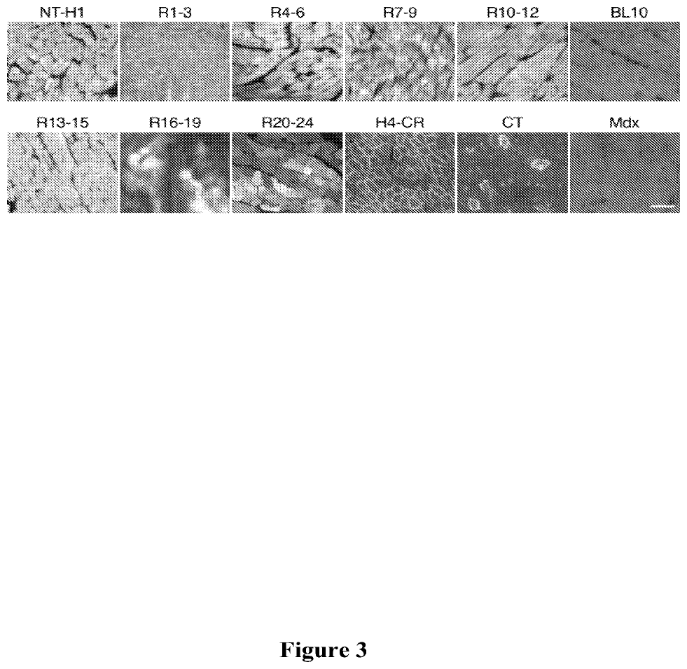

It is well established that dystrophin interacts with a congregation of cellular proteins (FIG. 3) (Johnson, E. K. et al. PLoS One 8, e73224 (2013); Johnson, E. K. et al. PLoS One 7, e43515 (2012); Allen, D. G. et al. Physiol. Rev. 96, 253-305 (2016); Constantin, B. Dystrophin complex functions as a scaffold for signaling proteins. Biochim. Biophys. Acta 1838, 635-642 (2014); Gao, Q. Q. & McNally, E. M. Compr Physiol 5, 1223-1239 (2015)). Besides the well known dystrophin-associated glycoprotein complex (DGC) (which includes dystroglycans, nNOS, syntrophin, dystrobrevins, sarcoglycans and sarcospan), dystrophin also interacts with cytoskeleton proteins (such as actin, tubulin, keratin, synemin and plectin), signaling proteins (such as Grb2, PAR-1b, cypher and ahnak1), channel proteins (such as TRPC1, TRPC4 and Nav1.5), caveolae proteins (such as caveolin-3 and cavin-1), tripartite motif proteins (e.g. myospryn) and chaperones (e.g. CRYAB). R10-12 belongs to the second actin-binding domain of dystrophin, and the CT-domain has the syntrophin and dystrobrevin binding motifs (Sadoulet-Puccio, et al. Proc. Natl. Acad. Sci. USA 94, 12413-12418 (1997). In certain embodiments provided herein, protein binding determinants in R10-R12 (F-actin), R16-R17 (nNOS), CR (beta-dystroglycan), and/or in CT (sarcoglycan, dystrobrevin, syntropin) are retained in the synthetic mini and micro dystrophin proteins and nucleic acids encoding the same that are provided herein.

In certain embodiments, the synthetic nucleic acid molecules provided herein comprise membrane binding motifs or membrane binding domains from the R10-R11-R12 regions of dystrophin that can be coupled with at least two membrane binding motifs or membrane binding domains from the R1-R2-R3, CR, and CT regions of dystrophin protein.

Membrane binding motifs of the R1-R2-R3 region used in the synthetic mini or micro dystrophins provided herein include, but are not limited to, the S-palmitoylation site peptide of SEQ ID NO: 54, SEQ ID NO: 55, and SEQ ID NO:56. In certain embodiments, the membrane binding domain of the R1-R2-R3 region used in the synthetic mini or micro dystrophins comprises the R1 repeat or the R1 and the R2 repeats.

Membrane binding motifs of the R10-R11-R12 region used in the synthetic mini or micro dystrophins provided herein include, but are not limited to, the S-palmitoylation site peptide of SEQ ID NO:57. In certain embodiments, the membrane binding domains of R10-R11-R12 can comprise any one of the R10 repeat, the R11 repeat, the R12 repeat, the R10-R11 repeats, the R11-R12, or the R10 and R12 repeats.

Membrane binding motifs of the CT domain used in the synthetic mini or micro dystrophins provided herein include, but are not limited to, the MBM of the CT MBD comprises residues 3422 to 3535 of SEQ ID NO: 1 or residues 3501 to 3685 of SEQ ID NO:1.

In certain embodiments, the synthetic nucleic acid molecules provided herein can comprise a nNOS binding domain of R16-R17. Such nNOS binding domains of the R16-R17 domains can comprise an R16-R17 peptide wherein the N-terminal alpha-helix of R16 (i.e., the sequence PSTYLTEITHVSQALLEVEQL (SEQ ID NO: 59) has been deleted where alpha-helices 2 and 3 of both of R16 and R17 are present. In certain embodiments, the N-terminal helix one of the R16 domain is substituted with the MBM of the R1-R2-R3 MBD or with the MBM of the R10-R11-R12 MBD. The remaining alpha-helices 2 and 3 of both of R16 and R17 along with the alpha-helix 1 of R17 that binds nNOS binding alpha-helix in vitro are sufficient to provide for in vivo nNOS binding (Lai, Y., et al., Proc. Natl. Acad. Sci. USA 110, 525-530 (2013).

In certain embodiments, the aforementioned dystrophin NT domain, repeats (e.g., R1, R2, R3, R10, R11, R12, R16, R17), CR domain, and CT domain are operably linked with a hinge region selected from the group consisting of a synthetic hinge, a semi-synthetic hinge, dystrophin H1, dystrophin H2, dystrophin H3, dystrophin H4, and variants thereof. A synthetic hinge can comprise or consist of one, two, or three, four, five or more “Gly-Gly-Ser-Gly” (SEQ ID NO:62) units. Other useful synthetic hinges that can be used include, but are not limited to: (i) [Gly-Ser]x linkers where x=2-10; (ii) one, two, or three, four, five or more “Gly-Gly-Gly-Ser” (SEQ ID NO:63) units; (iii) one, two, or three, four, five or more “Gly-Gly-Gly-Gly-Ser” (SEQ ID NO:64) units; (iv) one, two, or three, four, five or more “Ser-Glu-Gly” units; (v) one, two, or three, four, five or more “Gly-Ser-Ala-Thr” (SEQ ID NO:65) units; and (vi) any combination of (i)-(v) and/or of one, two, or three, four, five or more “Gly-Gly-Ser-Gly” (SEQ ID NO:62) units. A semi-synthetic hinge can comprise a dystrophin H1, H2, H3, or H4 hinge or portion thereof that incorporates a synthetic hinge.

Nucleic acids that encode the aforementioned syntrophin PDZ domain and/or dystrophin NT domain, repeats (e.g., R1, R2, R3, R10, R11, R12, R16, R17), CR domain, and CT domain that can be used include, but are not limited to, the nucleic acids provided in the sequence listing provided herein as well as by degenerate versions of those sequences that encode the same dystrophin polypeptide sequences. In certain embodiments, synthetic nucleic acids provided herein encode variants of the sequences of the aforementioned syntrophin PDZ domain and/or dystrophin NT domain, repeats (e.g., R1, R2, R3, R10, R11, R12, R16, R17), CR domain, and CT domain, or polypeptides contained therein that are listed in the sequence listing provided herewith or that are encoded by the nucleic acids listed in the sequence listing that: (i) exhibit at least 85%, 90%, 95%, 98%, or 99% sequence identity to the polypeptide sequence or encoded polypeptide sequence; (ii) contain 1, 2, 3, 4, 5, 6, or 7 conservative amino acid substitutions, insertions, or deletions; or (iii) incorporate one or more allelic variants of the sequence found in individuals with functional syntrophin PDZ domain or dystrophin genes that do not exhibit disease associated with loss or reductions in syntrophin PDZ domain or dystrophin activity.

In certain embodiments, the present disclosure provides vectors that can deliver the synthetic nucleic acid molecules encoding the micro or mini dystrophins or other fusion proteins provided herein. Any vector suitable for the purpose is contemplated by the present disclosure. In particular, the present disclosure provides a series of recombinant adeno-associated viral vectors (AAVs) and lentiviral vectors to deliver the nucleic acid molecules of the present disclosure (mini/micro-dystrophin genes) that exhibit improved membrane binding and biological activity. In certain embodiments, recombinant AAV vector (single vector or dual vectors) in accordance with the present disclosure includes any one of the nucleic acid molecule of the present disclosure (the mini/micro-dystrophin genes) that exhibit improved membrane binding and biological activity, operably linked to an expression cassette (a promoter and a polyA) and viral inverted terminal repeats (ITRs).

Numerous expression cassettes and vectors can be used with the micro and minidystrophin genes provided herein. By “expression cassette” is meant a complete set of control sequences including, but not limited to, initiation, promoter and termination sequences which function in a cell when they flank a structural gene in the proper reading frame. Expression cassettes frequently contain an assortment of restriction sites suitable for cleavage and insertion of any structural gene, e.g., the microgene or minigene of the present disclosure. In certain embodiments, the cloned gene will have a start codon in the correct reading frame for the structural synthetic dystrophin-encoding sequence. In addition, the expression cassette for the present disclosure can in certain embodiments includes, but not limited to, a constitutive promoter sequence, e.g., a CMV, RSV, CMV, SV40, CAG, CK6, or MCK promoters, at one end to cause the gene to be transcribed, and a poly-A recognition sequence at the other end for proper processing and transport of the messenger RNA. Examples of such a useful (empty) expression cassette into which the microgene of the present disclosure can be inserted are pcis.RSVmcs, pcis.CMVmcs, pcis.CMVmcs-intron, pcis.SV40mcs, pcis.SV40mcs-intron, pcis.CK6mcs, and pcis.CAGmcs as described in Yue et al (Yue & Duan 2002 Biotechniques 33(3):672-678). Examples of such a useful (empty) expression cassette into which the minigene of the present disclosure can be inserted are pDD188, pDD293 and pDD295 as described in Duan et al (Duan, Yue and Engelhardt 2003 Methods in Molecular Biology 219:29-51) and pAG15, and pAG21 as described in Ghosh et al (Ghosh, Yue, Lai and Duan 2008 Molecular Therapy 16:124-130). In certain embodiments, the expression cassette will provide for a muscle-specific promoter that is operably linked to the nucleic acid encoding the synthetic dystrophin. In certain embodiments, a muscle creatine kinase (MCK) promoter or variant thereof that retains muscle-specific activity is operably linked to the nucleic acid encoding the synthetic dystrophin (Wang et al.; Gene Ther. 2008 November; 15(22):1489-99). In certain embodiments, a muscle creatine kinase, troponin I, a skeletal alpha-actin, a desmin muscle-specific promoter or a derivative or chimera thereof is used (US20110212529, incorporated herein by reference in its entirety with respect to these promoters). Other useful muscle-specific promoters that can be used include, but are not limited to, CK5, CK6, CK7, CK8, myoglobin, CSK, Pitx3, and HAS promoters, derivatives thereof, or chimeras thereof. Other useful expression cassettes that can be used in certain vectors in conjunction with the mini and microdystrophin gene expression cassettes include, but are not limited to, expression cassettes that incorporate one or more selectable marker genes, such as a kanamycin, chlorosulfuron, phosphonothricin, hygromycin, or methotrexate resistance gene.

The term “vector” refers to a DNA or RNA sequence which is able to replicate and express a foreign gene in a host cell. Typically, vector has one or more endonuclease recognition sites which can be cut in a predictable fashion by use of the appropriate enzyme. Such vectors are can further comprise additional structural gene sequences imparting markers for identifying and separating transformed cells. Useful markers/selection agents include, but are not limited to, kanamycin, chlorosulfuron, phosphonothricin, hygromycin and methotrexate. A cell in which the foreign genetic material in a vector is functionally expressed has been “transformed” by the vector and is referred to as a “transformant.” Useful vectors include, but are not limited to, a nAAV vector, by which is a single-stranded DNA molecule which derives from the genome of Adeno-associated viruses but is non-pathogenic.

The expression cassette containing a minigene or microgene operably linked to the control sequences can be ligated into a suitable vector for delivery. In certain embodiments, AAV and lentiviral vectors containing replication and control sequences compatible with the host cell are used. A suitable vector, such as a single AAV vector will typically carry viral inverted terminal repeats (ITR) at the ends, the promoters, and microgene and polyA site.

By “dual vector system” meant a vector system composed of two vectors, e.g., AAV vectors, in which system both vector carry a part of a gene or sequence to be delivered and the entire gene is reconstituted by interaction between the two vectors. In one embodiment, the two vectors of dual vector system, e.g., AAV dual vector system, of the present disclosure are trans-splicing vectors (ts vectors, e.g., tsAAV vectors). In another embodiment, the two vectors of dual vector system, e.g., AAV dual vector system, of the present disclosure are hybrid vectors (e.g., hybrid AAV vectors). Trans-splicing AAV vectors typically carry (in addition to what are presented in a single AAV vector) a splicing donor signal and a splicing acceptor signal. Hybrid AAV vector will typically carry (in addition to what are presented in a single AAV vector and in the trans-splicing vector) a homologous overlapping sequence, such as from the middle one-third of human placental alkaline phosphotase gene. A lentiviral vector will typically carry the 5′ long terminal repeats (LTR), the 3′ LTR and the packaging signal.

By “operably linked” is meant that a nucleic acid molecule or polypeptide is placed in a functional relationship with another nucleic acid molecule or polypeptide. For example, expression cassette (a promoter and a polyA) is operably linked to a mini/micro-dystrophin gene if the expression cassette provided for transcription and polyadenylation of the sequence.

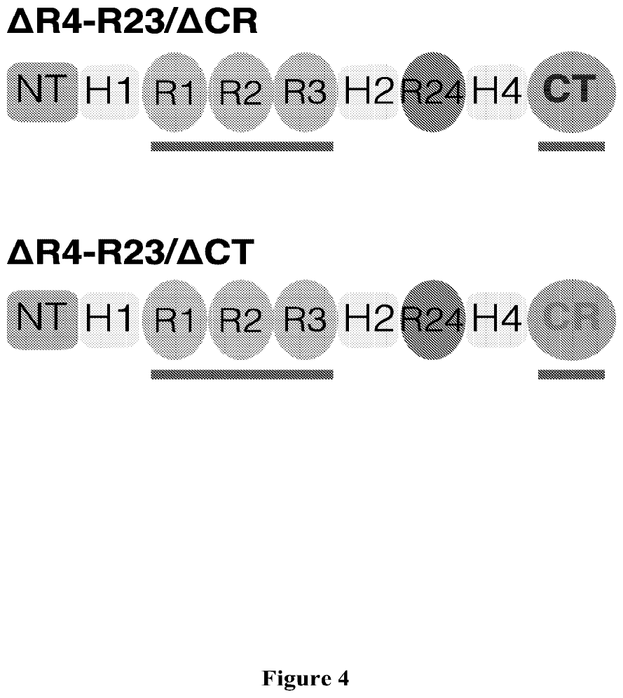

Dual AAV vectors of the present disclosure have large, e.g., at least 10 kb, packaging capacity. Three classical dual vectors are the cis-activation, trans-splicing (ts) and overlapping vectors (reviewed in Duan, D., Z. Yan, and J. F. Engelhardt. 2006. Expanding the capacity of AAV vectors, p. pp 525-32. In M. E. Bloom, S. F. Cotmore, R. M. Linden, C. R. Parrish, and J. R. Kerr (ed.), Parvoviruses. Hodder Arnold; Distributed in the U.S.A. by Oxford University Press, London, New York. Ghosh, A., and D. Duan. 2007. Expending Adeno-associated Viral Vector Capacity: A Tale of Two Vectors. Biotechnology and Genetic Engineering Reviews 24: 165-177, 2007.) The ts and overlapping vectors can deliver the 6 kb minigene. In tsAAV, a large therapeutic gene is split into a donor vector and an acceptor vector. The donor vector carries the 5′ part of the gene and a splicing donor signal. The acceptor vector carries a splicing acceptor signal and the 3′ part of the gene. Expression is achieved by AAV inverted terminal repeat (ITR)-mediated intermolecular recombination and subsequent splicing of the recombinant genome (FIG. 4) See Duan, D., Y. Yue, and J. F. Engelhardt. 2001. Expanding AAV Packaging Capacity With Transsplicing Or Overlapping Vectors: A Quantitative Comparison. Mol Ther 4:383-91, Sun, L., J. Li, and X. Xiao. 2000. Overcoming adeno-associated virus vector size limitation through viral DNA heterodimerization. Nat. Med. 6:599-602, and Yan, Z., Y. Zhang, D. Duan, and J. F. Engelhardt. 2000. From the Cover: Trans-splicing vectors expand the utility of adeno-associated virus for gene therapy. Proc. Natl. Acad. Sci. USA 97:6716-6721.

In the overlapping vectors, a large therapeutic gene is split into an upstream vector and a downstream vector. The upstream and the downstream vectors share a region of homology (Duan, D., Y. Yue, and J. F. Engelhardt. 2001., Halbert, C. L., J. M. Allen, and A. D. Miller. 2002. Efficient mouse airway transduction following recombination between AAV vectors carrying parts of a larger gene. Nat Biotechnol 20:697-701.) Transgene reconstitution is achieved through homologous recombination (FIG. 4). By rational vector design, such as optimizing the gene splitting site, the transduction efficiency from tsAAV vectors can reach that of a single AAV vector (Lai et al 2005 Nature Biotechnique; Lai et al 2006 Human Gene Therapy). Furthermore, systemic delivery of the tsAAV vectors has been shown to efficiently transduce whole body muscle in rodents (Ghosh, Yue, Long, Bostic and Duan 2007 Molecular Therapy 16:124-130). tsAAV-mediated minigene therapy was demonstrated to reduce muscle pathology, improve muscle force and prevent contraction-induced injury in a single mdx muscle (Lai, Y., D. Li, Y. Yue, and D. Duan. 2007. Design of trans-splicing adeno-associated viral vectors for Duchenne muscular dystrophy gene therapy. Method in Molecular Medicine: In-press., Lai, Y., Y. Yue, M. Liu, and D. Duan. 2006. Synthetic intron improves transduction efficiency of transsplicing adeno-associated viral vectors. Hum Gene Ther 17:1036-42, and Lai, Y., Y. Yue, M. Liu, A. Ghosh, J. F. Engelhardt, J. S. Chamberlain, and D. Duan. 2005. Efficient in vivo gene expression by trans-splicing adeno-associated viral vectors. Nat Biotechnol 23:1435-9.)

Besides the classic dual AAV vectors, a hybrid AAV dual vector system has been developed recently (Ghosh, Yue, Lai and Duan 2008 Molecular Therapy 16:124-130). The tsAAV is highly dependent on the optimal gene splitting site. This limitation is overcome in the hybrid vector system. In hybrid AAV vectors, transgene reconstitution can be achieved either through the traditional trans-splicing pathway as described in the tsAAV vectors or through homologous recombination via a highly recombinogenic foreign DNA sequence.

Accordingly, in still another embodiment, the present disclosure is directed to a method for the treatments of DMD, BMD and/or XLDC in a subject by administering to the subject a therapeutically effective amount of the minigene and/or microgene of the present disclosure, by administering a vector carrying the minigene and/or microgene, by administering to the subject a therapeutically effective amount of a AAV vector containing the minigene and/or microgene of the present disclosure. The term “subject” refers to any mammalian (e.g., human) or avian subject.

One route of the administration accordance with the method of the present disclosure includes, but is not limited to, local or regional muscle injection or forms of delivery to improve local muscle function in patients, systemic delivery (such as intravenous, intra-artery, intraperitoneal) to all or most muscles in a region or in the whole body in patients, in vitro infection of myogenic stem cells with AAV or lentiviral vector followed by local and/or systemic delivery.

By “therapeutically effective amount” is meant an amount high enough to significantly positively modify the condition to be treated but low enough to avoid serious side effects (at reasonable benefit/risk ratio) within the scope of sound medical judgment. The therapeutically effective amount will vary with the particular condition being treated, or the condition of the subject being treated and his/her physical condition, as well as the type of preparation, vector, or composition being used.

In a particular embodiment, the present disclosure contemplates intravascular administration. For example, in AAV-9 gene therapy with micro-dystrophin gene containing R16 and R17, the dosage to newborn mice (1 week or younger in age) is about 0.5 to about 1.5.times.10e11 vg particles/gram body weight or about 50 to about 75 .mu.l/gram body weight; the dosage to young mice (1 week to 1 month in age) is about 0.5 to about 1.5.times.10e11 vg particles/gram body weight or about 75 to about 200 .mu.l/gram body weight; the dosage to adult mice (1 to 20-month-old) is about 0.5 to about 1.5.times.10e11 vg particles/gram body weight or about 200 to about 400 .mu.l/gram body weight; the dosage for newborn dog (three days or younger in age) is about 0.5 to about 2.times.10e11 vg particles/gram body weight or about 10 to about 25 .mu.l/gram body weight; the dosage for young dog (3 days to 3 months in age) is about 0.5 to about 2.times.10e11 vg particles/gram body weight or about 10 to about 25 .mu.l/gram body weight; the dosage for adult dog (3-month-old or older) is about 1 to about 3.times.10e11 vg particles/gram body weight or about 15 to about 30 .mu.l/gram body weight.

According to the present disclosure, after engineering the membrane binding motifs or membrane binding domains of the R10-R11-R12 repeat into the mini/micro dystrophin protein encoding sequence, the resultant synthetic nucleic acid molecule can be incorporated into non-viral and/or viral gene therapy vectors, and/or cell therapy for the treatment of dystrophin deficient diseases such as DMD, BMD and XLDC. The present disclosure provides a series of AAV mini/micro-dystrophin vectors that can exhibit improved membrane binding and biological activity in a dystrophin-deficient muscle. An recombinant AAV vector includes, but is not limited to, any one of the mini/micro-dystrophin genes provided herein, an expression cassette (a promoter and a polyA), and viral inverted terminal repeats (ITRs).

In yet another embodiment, the present disclosure is directed to a pharmaceutical composition containing one or more of the AAV vectors and lentiviral vectors of the present disclosure and unmodified plasmid DNA molecules and a pharmaceutically acceptable carrier.

Pharmaceutical formulations, dosages and routes of administration for nucleic acids are generally disclosed, for example, in U.S. Pat. No. 5,580,859 to Felgner et al. Both local and systemic administration are contemplated by the present disclosure. In certain embodiments where the molecules of the disclosure are employed for prophylactic purposes, agents of the disclosure are amenable to chronic use, such as by systemic administration. One or more suitable unit dosage forms comprising the therapeutic agents of the disclosure, which can optionally be formulated for sustained release, can be administered by a variety of routes including, but not limited to, oral, parenteral, including by rectal, transdermal, subcutaneous, intravenous, intramuscular, intraperitoneal, intrathoracic, intrapulmonary, and intranasal routes. The formulations can, where appropriate, be conveniently presented in discrete unit dosage forms and can be prepared. Such methods can include the step of bringing into association the synthetic dystrophin encoding nucleic acid or synthetic dystrophin with liquid carriers, solid matrices, semi-solid carriers, finely divided solid carriers or combinations thereof, and then, optionally, introducing or shaping the product into the delivery system.

In certain embodiments where a synthetic dystrophin encoding nucleic acid, synthetic dystrophins, or vectors comprising or encoding the same are prepared for oral administration, they can be combined with a pharmaceutically acceptable carrier, diluent or excipient to form a pharmaceutical formulation, or unit dosage form.

By “pharmaceutically acceptable” is meant the carrier, diluent, excipient, and/or salt is compatible with the other ingredients of the formulation, and not deleterious to the recipient thereof. The active ingredient for oral administration can be present as a powder or as granules; as a solution, a suspension or an emulsion; or in achievable base such as a synthetic resin for ingestion of the active ingredients from a chewing gum. The active ingredient can also be presented as a bolus, electuary or paste.

Pharmaceutical formulations containing the a therapeutic agent of this disclosure including, but not limited to, synthetic dystrophin encoding nucleic acids, synthetic dystrophins, vectors or viral vector particle comprising or encoding the same, can be prepared. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The therapeutic agents of the disclosure can also be formulated as elixirs or solutions for convenient oral administration or as solutions appropriate for parenteral administration, for instance by intramuscular, subcutaneous or intravenous routes.

The pharmaceutical formulations of the therapeutic agents of the disclosure can also take the form of an aqueous or anhydrous solution or dispersion, or alternatively the form of an emulsion or suspension.

Thus, the therapeutic agent of this disclosure can be formulated for parenteral administration (e.g., by injection, for example, bolus injection or continuous infusion) and can be presented in unit dose form in ampules, pre-filled syringes, small volume infusion containers or in multi-dose containers with an added preservative. The active ingredients can take such forms as suspensions, solutions, or emulsions in oily or aqueous vehicles, and can contain formulatory agents such as suspending, stabilizing and/or dispersing agents. Alternatively, the active ingredients can be in powder form, obtained by aseptic isolation of sterile solid or by lyophilization from solution, for constitution with a suitable vehicle, e.g., sterile, pyrogen-free water, before use.

The compositions according to the disclosure can also contain thickening agents such as cellulose and/or cellulose derivatives. They can also contain gums such as xanthan, guar or carbo gum or gum arabic, or alternatively polyethylene glycols, bentones and montmorillonites, and the like.

In certain embodiments, an adjuvant chosen from antioxidants, surfactants, other preservatives, film-forming, keratolytic or comedolytic agents, perfumes and colorings can be added to the composition. Also, other active ingredients can be added, whether for the conditions described or some other condition.

The local delivery of the pharmaceutical composition of the present disclosure can also be by a variety of techniques which administer the agent at or near the site of disease. Examples of site-specific or targeted local delivery techniques are not intended to be limiting but to be illustrative of the techniques available. Examples include local delivery catheters, such as an infusion or in-dwelling catheter, e.g., a needle infusion catheter, shunts and stents or other implantable devices, site specific carriers, direct injection, or direct applications.

In particular, for delivery of a vector of the disclosure to a tissue such as muscle, any physical or biological method that will introduce the vector into the muscle tissue of a host animal can be employed. Vector means both a bare recombinant vector and vector DNA packaged into viral coat proteins to form a viral vector particle. Simply dissolving an AAV vector in phosphate buffered saline (PBS) or in N-2-Hydroxyethylpiperazine-N′-2-ethanesulfonic acid (HEPES) buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). The pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The vectors can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. In certain embodiments, such aqueous solutions can be buffered and the liquid diluent first rendered isotonic with saline or glucose. Solutions of the synthetic nucleic acid or vector as a free acid (DNA contains acidic phosphate groups) or a pharmacologically acceptable salt can be prepared in water suitably mixed with a surfactant such as hydroxypropylcellulose. A dispersion of AAV viral particles can also be prepared in glycerol, liquid polyethylene glycols and mixtures thereof and in oils. Under ordinary conditions of storage and use, these preparations contain a preservative to prevent the growth of microorganisms.

In certain embodiments, the Pharmaceutical forms or compositions suitable for injectable use include, but are not limited to, sterile aqueous solutions or dispersions and sterile powders for the extemporaneous preparation of sterile injectable solutions or dispersions. In certain embodiments, the form is sterile and fluid to the extent that easy syringability exists. It is typically stable under the conditions of manufacture and storage and is preserved against the contaminating action of microorganisms such as bacteria and fungi. In certain embodiments, the carrier can be a solvent or dispersion medium containing, for example, water, ethanol, polyol (for example, glycerol, propylene glycol, liquid polyethylene glycol and the like), suitable mixtures thereof, and vegetable oils. In certain embodiments, the proper fluidity can be maintained, for example, by the use of a coating such as lecithin, by the maintenance of a given particle size in the case of a dispersion and by the use of surfactants. In certain embodiments, the prevention of the action of microorganisms can be brought about by various antibacterial and antifungal agents that include, but are not limited to, parabens, chlorobutanol, phenol, sorbic acid, thimerosal and the like. In certain embodiments, isotonic agents, for example, sugars or sodium chloride are included. Prolonged absorption of the injectable compositions can be brought about by use of agents delaying absorption, for example, aluminum monostearate and gelatin.

In certain embodiments, sterile injectable solutions are prepared by incorporating the synthetic nucleic acid or vector in the desired amount in the appropriate solvent with various of the other ingredients enumerated above, followed by filtered sterilization. In certain embodiments, dispersions are prepared by incorporating the sterilized active ingredient into a sterile vehicle which contains the basic dispersion medium and the other ingredients from those enumerated above. In the case of sterile powders for the preparation of sterile injectable solutions, the preferred methods of preparation are vacuum drying and the freeze drying technique which yield a powder of the active ingredient plus any additional ingredient from the previously sterile-filtered solution thereof.

Also provided herein are methods and resultant host cells wherein a defective endogenous dystrophin gene of the host cell or a defective portion thereof is edited to provide the synthetic nucleic acid molecule within the host cell's X-chromosome. Such methods of gene editing include, but are not limited to, those that employ a clustered regularly interspaced short palindromic repeats (CRISPR)-associated (Cas)-guide RNA or source thereof and a Cas endonuclease or source thereof, wherein the guide RNA and Cas endonuclease can form a complex that can introduce a double strand break at a target site in a nuclear genome of the host cell that provides for incorporation of the synthetic nucleic acid or portion thereof into the endogenous dystrophin locus. Methods that can be adapted for this purpose are disclosed in US Patent Application publications US20160175462, US20160115488, and US20160153004, which are each incorporated herein by reference in their entireties.

### Abbreviations

DMD: Duchenne muscular dystrophy
CR: Cysteine-rich
NT: N-terminus
CT: C-terminus
R: Spectrin-like repeat
DGC: Dystrophin-associated glycoprotein complex
ECM: Extracellular matrix
H: Hinge region
MBD: Membrane binding domain
GFP: Green fluorescent protein
TA: Tibialis anterior
AAV: Adeno-associated virus

To the extent to which any of the preceding abbreviations or definitions is inconsistent with abbreviations or definitions provided in any patent or non-patent reference incorporated herein by reference, any patent or non-patent reference cited herein, or in any patent or non-patent reference found elsewhere, it is understood that the preceding definition will be used herein.

Non-limiting embodiments provided herein include:

Embodiment 1. A synthetic nucleic acid molecule encoding a synthetic mini-dystrophin gene or micro-dystrophin gene encoding a synthetic, non-full length dystrophin protein comprising: (i) an N-terminal (NT) domain of the dystrophin protein or a modified N-terminal domain of the dystrophin protein; (ii) at least two membrane binding motifs (MBM) independently selected from the group consisting of an MBM of an R1-R2-R3 membrane binding domain (MBD), an MBM of a CR membrane binding domain, and an MBM of a CT membrane binding domain; (iii) an MBM of an R10-R11-R12 MBD; and (iv) an nNOS binding domain of R16-R17; wherein the domains and the MBM are arranged from N to C terminus in the order in which they occur in a wild-type dystrophin protein and are operably linked.

Embodiment 2. The synthetic nucleic acid molecule of embodiment 1, wherein the MBM of R1-R2-R3 comprises at least one S-palmitoylation site peptide selected from the group consisting of SEQ ID NO: 54, SEQ ID NO: 55, and SEQ ID NO:56.

Embodiment 3. The synthetic nucleic acid molecule of embodiment 1, wherein R3 repeat or R2-R3 repeats are absent from the non-full length dystrophin protein.

Embodiment 4. The synthetic nucleic acid molecule of embodiment 1, wherein the R1, R2, R3, R1 and R2, R2 and R3, or R1, R2, and R3 repeats are present in the non-full length dystrophin protein.

Embodiment 5. The synthetic nucleic acid molecule of embodiment 1, wherein the MBM of R10-R11-R12 comprises an S-palmitoylation site peptide of SEQ ID NO:57.

Embodiment 6. The synthetic nucleic acid molecule of embodiment 1, wherein the R10 repeat, the R11 repeat, the R12 repeat, the R10-R11 repeats, the R11-R12, or the R10 and R12 repeats are present in the non-full length dystrophin protein.

Embodiment 7. The synthetic nucleic acid molecule of embodiment 1, wherein the R17 domain is present in the non-full length dystrophin protein.

Embodiment 8. The synthetic nucleic acid molecule of embodiment 1, wherein the n-terminal alpha helix of the R16 domain (SEQ ID NO:59) or a portion thereof is absent from the non-full length dystrophin protein.

Embodiment 9. The synthetic nucleic acid molecule of embodiment 8, wherein alpha-helix 2 and alpha-helix 3 of the R16 domain is present and alpha-helix 1, alpha-helix 2, and alpha-helix 3 of the R17 domain is present in the non-full length dystrophin protein.

Embodiment 10. The synthetic nucleic acid molecule of embodiment 8, wherein alpha-helix 2 and alpha-helix 3 of the R16 domain is present and alpha-helix 1, alpha-helix 2, and alpha-helix 3 of the R17 domain is present in the non-full length dystrophin protein.

Embodiment 11. The synthetic nucleic acid molecule of embodiment 8, wherein N-terminal helix one of the R16 domain is substituted with the MBM of the R1-R2-R3 MBD or with the MBM of the R10-R11-R12 MBD.

Embodiment 12. The synthetic nucleic acid molecule of embodiment 1, wherein the R16 domain and the R17 domain are present in the non-full length dystrophin protein.

Embodiment 13. The synthetic nucleic acid molecule of embodiment 1, wherein the MBM of the CR membrane binding domain is absent, wherein the CR membrane binding domain is absent, or wherein the CR domain is absent from the non-full length dystrophin protein.

Embodiment 14. The synthetic nucleic acid molecule of embodiment 1, wherein the MBM of the CT MBD comprises residues 3422 to 3535 of SEQ ID NO: 1.

Embodiment 15. The synthetic nucleic acid molecule of embodiment 1, wherein the MBM of the CT MBD comprises residues 3501 to 3685 of SEQ ID NO:1.

Embodiment 16. The synthetic nucleic acid of embodiment 1, wherein at least one domain and at least one MBM are operably linked with a hinge region selected from the group consisting of a synthetic hinge, a semi-synthetic hinge, dystrophin H1, dystrophin H2, dystrophin H3, dystrophin H4, and variants thereof.

Embodiment 17. The synthetic nucleic acid of embodiment 1, wherein the dystrophin H1 hinge or a variant thereof operably links the C-terminus of the NT domain to the N-terminus of an MBM or domain containing an MBM, wherein the dystrophin H2 hinge or a variant thereof operably links the C-terminus of a MBM or domain containing an MBM to the N-terminus of another MBM or domain containing another MBM, wherein the dystrophin H3 hinge or a variant thereof operably links the C-terminus of an MBM or domain containing an MBM to the N-terminus of another MBM or domain containing another MBM, wherein the dystrophin H4 hinge or a variant thereof operably links the C-terminus of an MBM to the N-terminus of the CR MBM or the CR domain, or any combination thereof.

Embodiment 18. The synthetic nucleic acid of embodiment 1, wherein the dystrophin H4 hinge or a variant thereof operably links the C-terminus of an MBM to the N-terminus of the CR MBM or the CR domain.

Embodiment 19. The synthetic nucleic acid molecule of any one of embodiments 1 to 18, wherein the mini- or micro-dystrophin gene is between 5 kb to about 8 kb in length or less than 5 kb in length, respectively.

Embodiment 20. The synthetic nucleic acid molecule of any one of embodiments 1 to 18, wherein the mini- or micro-dystrophin gene is operably linked to a heterologous promoter, a heterologous 5′ untranslated region (UTR), a heterologous 3′ UTR, a heterologous polyadenylation site, or any combination thereof.

Embodiment 21. The synthetic nucleic acid molecule of any one of embodiments 1 to 18, wherein said molecule is integrated within an endogenous dystrophin gene locus in an X-chromosome.

Embodiment 22. A lentiviral vector comprising the synthetic nucleic acid molecule of any one of embodiments 1 to 20, wherein the nucleic acid molecule is operably linked to an expression cassette, 5′ and 3′ long terminal repeats (LTR), and a psi sequence in the lentiviral vector.

Embodiment 23. A single recombinant adeno-associated virus (AAV) vector comprising the nucleic acid of any one of embodiments 1 to 20, wherein said nucleic acid molecule is operably linked to an expression cassette and viral inverted terminal repeats (ITRs) in the AAV.

Embodiment 24. A dual recombinant AAV vector system, comprising two AAV vectors, wherein one of the two AAV vectors comprises a part of the nucleic acid molecule of any one of embodiments 1 to 20, and the other vector comprises the remaining part of said nucleic acid molecule, wherein the two vectors further comprise sequences that permit recombination with each other to produce said nucleic acid in full length, and wherein the nucleic acid in full length is operably linked to an expression cassette and viral ITRs.

Embodiment 25. A composition comprising the synthetic nucleic acid molecule of any one of embodiments 1 to 20 and a pharmaceutically acceptable carrier.

Embodiment 26. The composition of embodiment 25, wherein the nucleic acid molecule is operably linked to an expression cassette, 5′ and 3′ long terminal repeats (LTR), and a psi sequence in a lentiviral vector.

Embodiment 27. The composition of embodiment 25, wherein said nucleic acid molecule is operably linked to an expression cassette and viral inverted terminal repeats (ITRs) in an AAV

Embodiment 28. The composition of embodiment 25 comprising the dual recombinant AAV vector system of embodiment 24.

Embodiment 29. An isolated host cell comprising the synthetic nucleic acid molecule of any one of embodiments 1 to 21.

Embodiment 30. The host cell of embodiment 29, wherein said nucleic acid molecule is integrated within an endogenous dystrophin gene locus in a chromosome of the host cell.

Embodiment 31. The host cell of embodiment 29, wherein the nucleic acid molecule is operably linked to an expression cassette, 5′ and 3′ long terminal repeats (LTR), and a psi element in a lentiviral vector.

Embodiment 32. The host cell of embodiment 29, wherein said nucleic acid molecule is operably linked to an expression cassette and ITRs in an AAV.

Embodiment 33. The host cell of embodiment 29, wherein the host cell is a myogenic stem cell.

Embodiment 34. A method for the treating or ameliorating one or more adverse effects of Duchenne muscular dystrophy (DMD), Becker muscular dystrophy (BMD) or X-linked dilated cardiomyopathy (XLDC) in a subject in need thereof comprising the step of administering to the subject a therapeutically effective amount of: (i) the synthetic nucleic acid molecule of any one of embodiments 1 to 21; (ii) the lentiviral vector of embodiment 22; (iii) the AAV vector of embodiment 23; (iv) the composition of any one of embodiments 25 to 28; or (iv) the host cell of any one of embodiments 29 to 33 to a subject in need thereof.

Embodiment 35. The method of embodiment 34, wherein the administration is by injection into muscle, systemic delivery, or local delivery.

Embodiment 36. The method of embodiment 34, wherein the host cell is a stem cell or myogenic stem cell.

Embodiment 37. The method of embodiment 34 or 36, wherein the host cell is derived from an autologous cell of the subject.

Embodiment 38. The method of any one of embodiments 34, 35, 36, or 37, wherein a defective endogenous dystrophin gene of the host cell or a defective portion thereof is edited to provide the synthetic nucleic acid molecule within the host cell's X-chromosome.

Embodiment 39. Use of (i) the synthetic nucleic acid molecule of any one of embodiments 1 to 21; (ii) the lentiviral vector of embodiment 22; (iii) the AAV vector of embodiment 23; (iv) the composition of any one of embodiments 25 to 28; or (iv) the host cell of any one of embodiments 29 to 33 for making a composition for administration to a subject suffering from Duchenne muscular dystrophy (DMD), Becker muscular dystrophy (BMD) or X-linked dilated cardiomyopathy (XLDC).

Embodiment 40. Use of (i) the synthetic nucleic acid molecule of any one of embodiments 1 to 21; (ii) the lentiviral vector of embodiment 22; (iii) the AAV vector of embodiment 23; (iv) the composition of any one of embodiments 25 to 28; or (iv) the host cell of any one of embodiments 29 to 33 for treating a subject suffering from Duchenne muscular dystrophy (DMD), Becker muscular dystrophy (BMD) or X-linked dilated cardiomyopathy (XLDC), or for ameliorating one or more adverse effects of DMD, BMD, or XLDC.

Embodiment 41. A synthetic nucleic acid molecule encoding a synthetic mini-dystrophin gene or micro-dystrophin gene encoding a synthetic, non-full length dystrophin protein comprising: (i) an N-terminal (NT) domain of the dystrophin protein or a modified N-terminal domain of the dystrophin protein; (ii) at least two membrane binding motifs (MBM) independently selected from the group consisting of an MBM of an R1-R2-R3 membrane binding domain (MBD), an MBM of a CR membrane binding domain, and an MBM of a CT membrane binding domain; (iii) an MBM of an R10-R11-R12 MBD; and (iv) an nNOS binding domain of R16-R17 or an nNOS binding domain of R16-R17 that is operably linked to a syntrophin PDZ domain; wherein the dystrophin domains and the MBM are arranged from N to C terminus in the order in which they occur in a wild-type dystrophin protein and are operably linked.

Embodiment 42. A synthetic nucleic acid molecule comprising a sequence encoding a fusion protein comprising a nNOS binding domain of dystrophin R16-R17 that is operably linked to a syntrophin PDZ domain.

Embodiment 43. A single recombinant adeno-associated virus (AAV) vector comprising the nucleic acid molecule of embodiment 41 or 42, wherein said nucleic acid molecule is operably linked to an expression cassette and viral inverted terminal repeats (ITRs) in the AAV.

Embodiment 44. A dual recombinant AAV vector system, comprising two AAV vectors, wherein one of the two AAV vectors comprises a part of the nucleic acid molecule of embodiment 41 or 42, and the other vector comprises the remaining part of said nucleic acid molecule, wherein the two vectors further comprise sequences that permit recombination with each other to produce said nucleic acid in full length, and wherein the nucleic acid in full length is operably linked to an expression cassette and viral ITRs.

Embodiment 45. A lentiviral vector comprising the synthetic nucleic acid molecule of embodiment 41 or 42, wherein the nucleic acid molecule is operably linked to an expression cassette, 5′ and 3′ long terminal repeats (LTR), and a psi sequence in the lentiviral vector.

Embodiment 46. A fusion protein comprising dystrophin nNOS binding domain of R16-R17 that is operably linked to a syntrophin PDZ domain.

Embodiment 47. A composition comprising (i) the synthetic nucleic acid molecule of embodiment 41 or 42, the vector of embodiment 43, 44, or 45, or the protein of embodiment 46; and (ii) a pharmaceutically acceptable carrier.

Embodiment 48. An isolated host cell comprising the synthetic nucleic acid molecule of embodiment 41 or 42, or the vector of embodiment 43, 44, or 45.

Embodiment 49. A method for the treating or ameliorating one or more adverse effects of Duchenne muscular dystrophy (DMD), age-related muscle atrophy, cancer cachexia, or other neuromuscular disorders characterized by loss of sarcolemmal neuronal nitric oxide synthase (nNOS) activity in a subject in need thereof comprising the step of administering to the subject a therapeutically effective amount of: (i) the synthetic nucleic acid molecule of any one of embodiments 41 or 42; (ii) the lentiviral vector of embodiment 45; (iii) the AAV vector of embodiment 43 or 44; (iv) the composition of embodiment 47; or (iv) the host cell of embodiment 48 to a subject in need thereof.

Embodiment 50. The method of embodiment 49, wherein the administration is by injection into muscle, systemic delivery, or local delivery.

### EXAMPLES

The following examples are included to demonstrate various embodiments. It will be appreciated by those of skill in the art that the techniques disclosed in the following examples represent techniques discovered by the Applicants to function well. However, those of skill in the art should, in light of the instant disclosure, appreciate that many changes can be made in the specific embodiments that are disclosed, while still obtaining like or similar results, without departing from the scope of the disclosure.

### Example 1. Identification of Dystrophin R1-3, R10-12 and CT as New Dystrophin MBDs

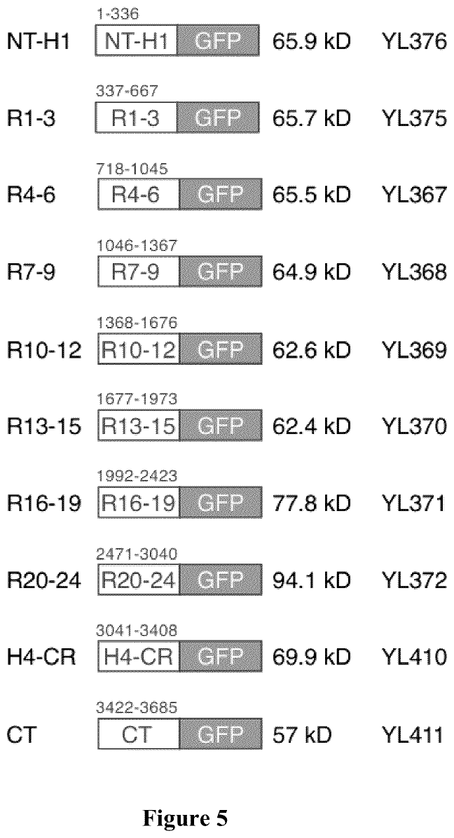

To thoroughly understand how dystrophin interacts with the sarcolemma, we performed a comprehensive screening in mouse muscle. According to the fact that dystrophin has four functional domains and its mid-rod domain can be further divided into sub-regions (14), we split the full-length human dystrophin protein into ten subdomains, including NT-H1, R1-3, R4-6, R7-9, R10-12, R13-15, R16-19, R20-24, H4-CR and CT. We fused each subdomain with a green fluorescent protein (GFP) tag and individually expressed them in the tibialis anterior (TA) muscle of dystrophin-null mdx mice by adeno-associated virus (AAV)-mediated gene transfer (FIG. 5).

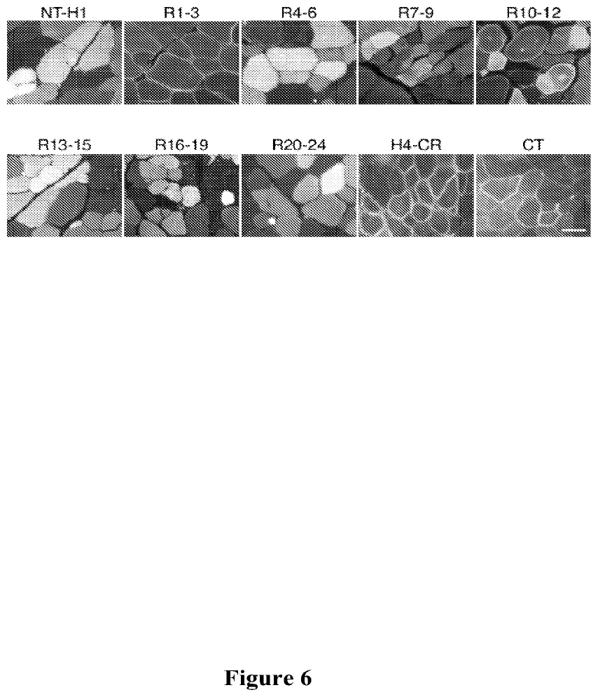

To determine subcellular localizations of each dystrophin subdomain, we visualized the GFP signal under a fluorescence microscope (FIG. 6). In line with the literature, we observed sarcolemmal localization of the H4-CR subdomain. Unexpectedly, we found that subdomains R1-3 and CT were exclusively restricted at the muscle cell membrane. Subdomains NT-H1, R4-6, R7-9, R13-15, R16-19, and R20-24 were only detected in the cytosol. Interestingly, the R10-12 subdomain was found both at the sarcolemma and in the cytoplasm (FIG. 6).

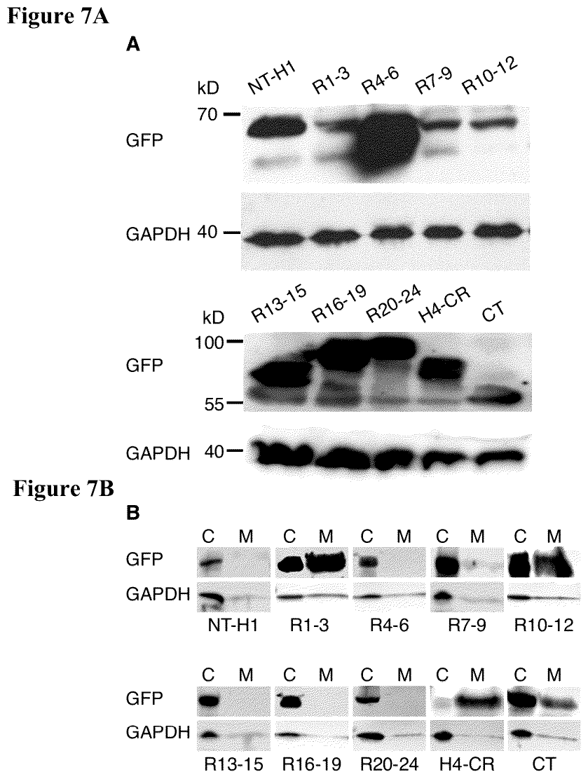

To confirm these intriguing observations, we performed immunoblot with whole muscle lysates and microsomal preparations (FIG. 7). In whole muscle lysates, we found efficient expression of all ten dystrophin subdomains (FIG. 7A). However, only subdomains R1-3, R10-12, CR and CT were detected in membrane-enriched microsomal preparations (FIG. 7B). These data are in agreement with immunostaining results suggesting that these subdomains are indeed dystrophin MBDs.

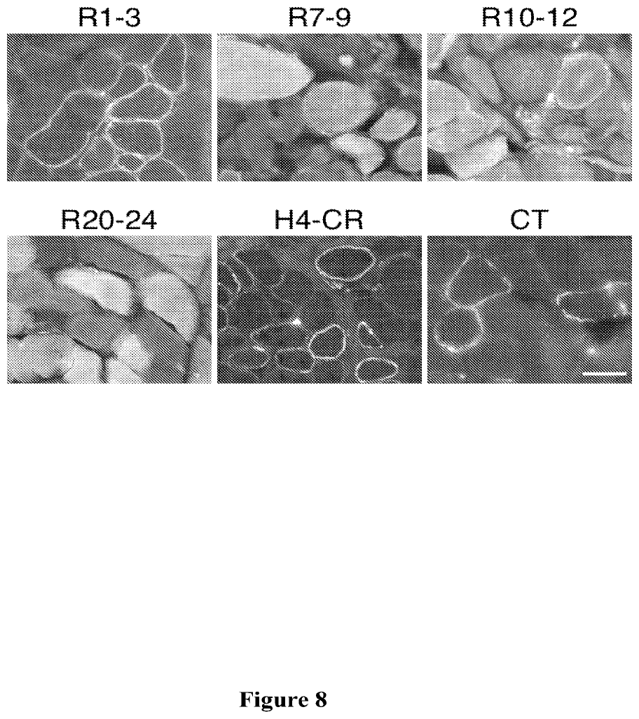

Preservation of the membrane-binding property of R1-3, R10-12, CR and CT in canine muscle. To examine whether the membrane-binding property of R1-3, R10-12, CR and CT is conserved in different species, next we delivered the corresponding AAV vectors to dystrophic dog muscle by local injection. As controls, we also injected R7-9 and R20-24 AAV vectors. Two months later, we examined GFP expression under a fluorescence microscope. Similar to what we saw in mdx muscle, R1-3, CR and CT subdomains were exclusively localized at the muscle membrane, while the R10-12 subdomain was found both at the sarcolemma and in the cytoplasm. Subdomains R7-9 and R20-24, which localized exclusively in the cytosol in mdx muscle, were only detected in the cytosol of dystrophic dog muscle (FIG. 8)

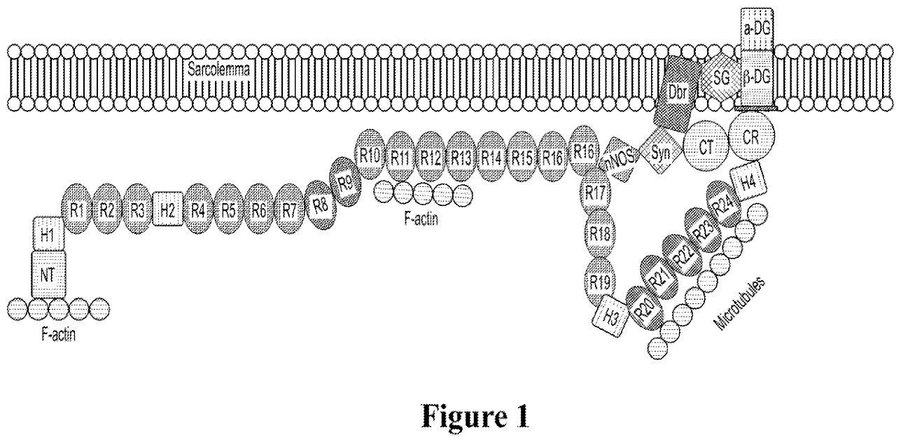

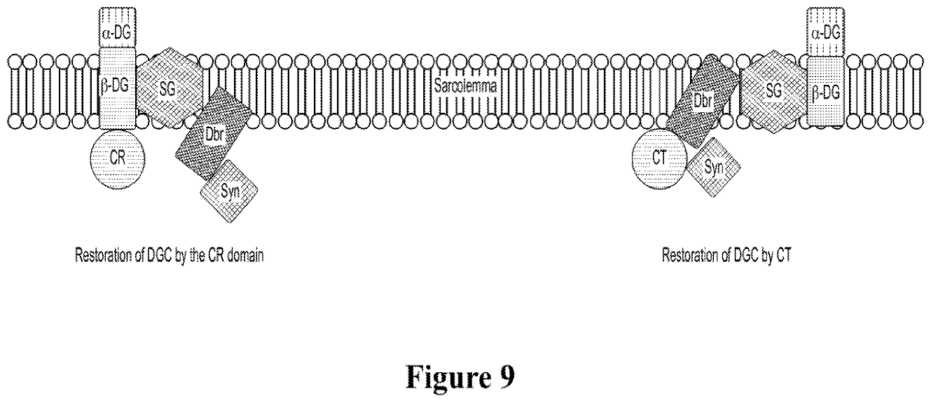

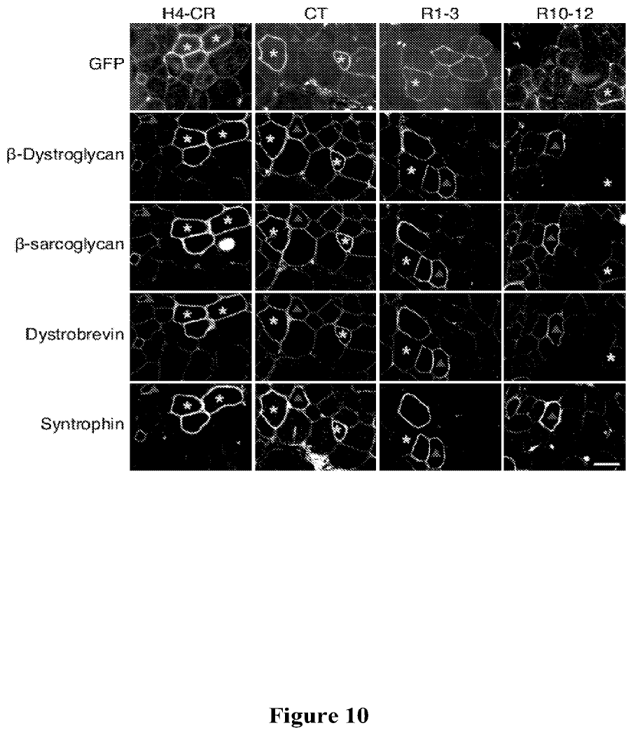

Independent restoration of the DGC by the CR domain and CT. In the canonical model (FIGS. 1 and 9), the CR domain is solely responsible for nucleating dystroglycan, sarcoglycans, dystrobrevin and syntrophin into the DGC at the sarcolemma (15-18). To determine whether the newly identified MBDs had similar functions, we evaluated DGC components on serial muscle sections by immunostaining (FIG. 10). As expected, the H4-CR subdomain successfully restored β-dystroglycan, β-sarcoglycan, dystrobrevin and syntrophin to the sarcolemma. Myofibers that were transduced with the CT subdomain AAV vector also resulted in sarcolemmal localization of these DGC components. In muscles infected with R1-3 and R10-12 AAV vectors, DGC components were detected in GFP-negative revertant fibers but not in transduced GFP-positive myofibers (FIG. 10).

Conservation of the membrane-binding property of R1-3, CR and CT in cardiac muscle. To determine whether our findings in skeletal muscle can be extended to cardiac muscle, we delivered GFP-fusion subdomain AAV vectors via the tail vein (FIG. 3). Compared with un-injected BL10 and mdx controls, systemic AAV injection resulted in robust GFP signals in the myocardium. Several different patterns were observed. The H4-CR subdomain was restricted at the sarcolemma while subdomains NT-H1, R4-6, R10-12, R13-15, R16-19 showed exclusive cytosolic expression. The R1-3 subdomain was found in the cytosol and the intercalated disk. In the mice infected with the CT-GFP AAV vector, we only detected a few GFP positive cardiomyocytes. Interestingly, GFP signals in these cells were found predominantly at the sarcolemma (FIG. 3).

### Discussion

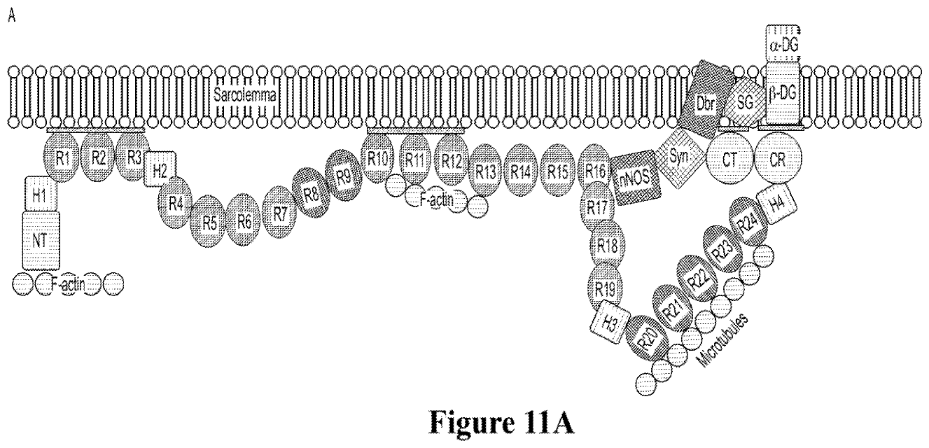

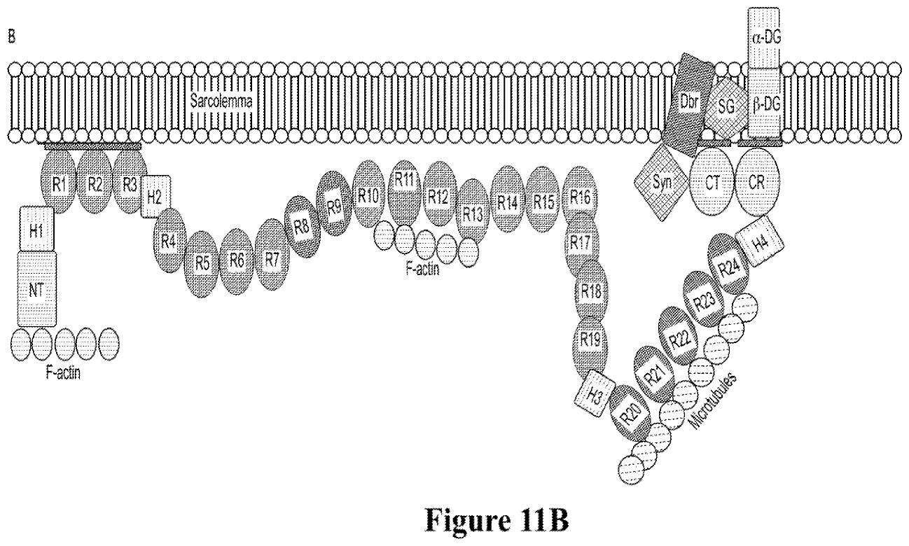

In this study, we performed the first comprehensive in vivo evaluation of the subcellular localizations of dystrophin subdomains. We demonstrated that in addition to the CR domain, dystrophin contains several highly conserved MBDs that can independently interact with the sarcolemma. These newly identified MBDs are R1-3, R10-12 and CT (FIG. 11). The CT subdomain bound to the sarcolemma in both skeletal muscle and cardiac muscle. Further it restored the DGC. Subdomain R1-3 showed exclusive membrane binding in skeletal muscle (FIG. 11A) but a preference for the intercalated disk in the heart (FIG. 11B). Subdomain R10-12 only demonstrated partial membrane localization in skeletal muscle (FIG. 11A).

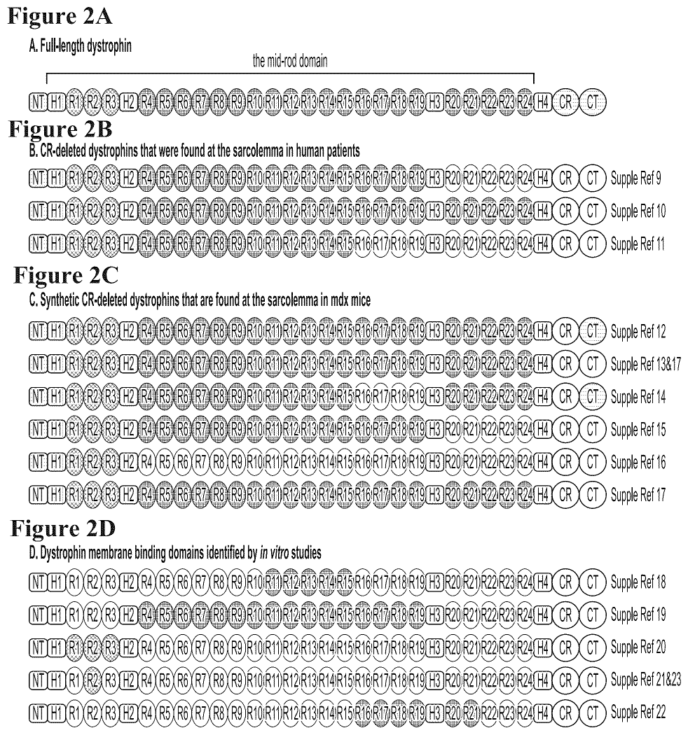

Interaction with the sarcolemma is central to how dystrophin protects muscle. A wealth of molecular, biochemical and structural studies has provided unequivocal proof that the CR domain anchors dystrophin to the sarcolemma via the formation of the DGC (7-9). Hence it has been quite puzzling why dystrophins that lack the CR domain still appear to bind to the sarcolemma in some atypical patients (11-13). Studies performed in mdx mice suggest that these puzzling patient observations can well be true. Of notice, forced expression of fragmented dystrophins that lack the CR domain has been repeatedly detected at the sarcolemma in mdx mice (FIG. 2C) (19-24). Collectively, it is reasonable to hypothesize that dystrophin can carry additional membrane localization domain(s).

To better understand dystrophin-sarcolemma interaction, investigators have turned to the artificial in vitro systems. These studies identified a number of potential regions capable of membrane binding such as R2, R1-3, R4-19, R11-15, R16-21 (FIG. 2D) (14, 25-30). Essentially, 21 out of 24 spectrin-like repeats in the rod domain were found to carry the membrane binding property in these in vitro studies. Such a broad range makes it almost impossible to pinpoint the identity of true dystrophin MBDs. Considering the fact that in vivo performance of dystrophin spectrin-like repeats cannot be accurately predicted by in vitro analysis (31), it becomes even more challenging to characterize the CR domain-independent dystrophin-sarcolemma interaction in test tubes. Here we took a systematic and unbiased approach with an emphasis on the in vivo interaction in rodents and large mammals. We found four structurally defined regions in dystrophin that are capable of interacting with the sarcolemma. These include the well-studied CR domain and three new MBDs (two in the rod domain and one in CT). While R1-3 and R10-12 have been implicated in some in vitro studies, direct binding of CT to the sarcolemma has never been reported. Intriguingly, CT also restores the DGC (FIG. 10). It is intriguing that we observed striking differences in the membrane binding behavior of the newly identified rod domain MBDs. Specifically, R1-3 is not restricted to the sarcolemma in the heart and R10-12 has no membrane binding activity in the heart (FIG. 3). This is reminiscent of different nNOS-binding properties of dystrophin in the muscle and the heart (32, 33). Collectively, these data suggest that dystrophin can have different functional roles in the muscle and the heart.

The mechanism(s) by which these newly identified MBDs bind to the sarcolemma await future investigations. It is possible that electrostatic and/or hydrophobic interactions can play a role. However, considering what is known about other spectrin family proteins, we suspect that such interactions can likely involve specified membrane domains (such as lipid rafts) and palmitoylation (34).

Restoration of the DGC by CT is another unexpected finding in this study. We speculate that CT can utilize its syntrophin/dystrobrevin binding motifs to recruit syntrophin and dystrobrevin first. Subsequently, these two proteins scaffold sarcoglycans and dystroglycan to the complex (FIG. 9) (35-38).

Another area that requires further analysis is the kinetic mode of interaction between different MBDs and the sarcolemma. A recent study in the zebrafish suggests that dystrophin can associate with the sarcolemma either via stable tight interaction or via reversible dynamic shuttling between the sarcolemma and the cytosol (39). While additional studies are needed, the results of our microsomal preparation western blot seem to hint that the CR domain is responsible for stable membrane binding (GFP signals were barely detected in the cytosolic fraction) and three newly discovered MBDs can contribute to dynamic membrane binding (abundant GFP signals also presented in the cytosol) (FIG. 7B).

There are a few limitations in our study. First, we have not included hinges 2 and 3 in our constructs. Due to the structural properties of hinges (proline-rich, neither α-helix nor (3-sheet), we suspect that these hinge regions can play a nominal role in membrane binding. Nevertheless, future studies are needed to confirm this. Second, we have used an over-expression system in our studies and also the fragmented dystrophin domains are not in their natural protein environment. It remains to be determined whether the membrane binding properties of the newly discovered MBDs are preserved under physiological concentration of dystrophin in wild type animals.

Taken together, we have discovered a new model for dystrophin membrane binding (FIG. 11). Our results offer insights into dystrophin function, DMD pathogenesis and gene therapy.

### Materials and Methods

Animals. All animal experiments were approved by the Animal Care and Use Committee of the University of Missouri, and the animal use and handling were strictly in accordance with the National Institutes of Health guidelines. Dystrophin-null mdx mice were purchased from The Jackson Laboratory (Bar Harbor, ME). Dystrophin-deficient dogs were generated in house by artificial insemination.

AAV production and delivery. The GFP gene was fused in-frame to the C-terminal ends of the human dystrophin subdomains (FIG. 5). The fusion constructs were cloned into the cis AAV packaging constructs by PCR and confirmed by sequencing. Expression was driven by the cytomegalovirus promoter and the SV40 poly-adenylation signal. Y731F AAV-9 vectors were generated by transient transfection and purified through two rounds of CsCl gradient ultracentrifugation (40, 41). The viral titer was determined by quantitative PCR.

AAV vectors were delivered by intramuscular injection to limb muscles to adult mdx mice (4-7×10¹¹ vg particles/muscle) and adult dystrophic dogs (0.8-4×10¹⁴ vg particles/muscle). In dog studies, we applied 5-week transient immune suppression with cyclosporine and mycophenolate mofetil according to our published protocol (42).

Muscle harvesting, microscopic examination and western blot. Eight weeks after injection, animals were euthanized and muscles were harvested according to Liadaki et al through serial sucrose gradient to preserve the GFP signal (43). GFP was visualized directly under the fluorescein isothiocyanate channel using a fluorescence microscope. Immunostaining was performed as we published before (31, 44). Whole muscle lysates were generated as we published before (31, 44). The cytosolic and microsomal preparations were obtained with the Plasma Membrane Protein Extraction kit (ab65400, Abcam). Muscle lysates were resolved in a 6% sodium dodecyl sulfate polyacrylamide gel and transferred to a polyvinylidene difluoride membrane. Antibodies used in immunostaining and western blot are listed in Table S1.

TABLE 1
Antibodies used in the study.

| Antigen | Host | Catalog # | Company | Dilution | Experiment |
|---|---|---|---|---|---|
| β-Dystroglycan | Mouse | NCL-B-DG | Novocastra | 1:50 | IF |
| Syntrophin | Mouse | ab11425 | Abcam | 1:200 | IF |
| β-Sarcoglycan | Mouse | NCL-B-SARC | Novocastra | 1:50 | IF |
| Dystrobrevin | Mouse | 610766 | BD Bioscience | 1:200 | IF |
| GFP | Mouse | 33-2600 | Invitrogen | 1:100 | WB |
| GAPDH | Mouse | MAB374 | Millipore | 1:5,000 | WB |

IF: Immunofluorescence staining; WB: western blot.

### Example 2. Molecular Mechanisms for Membrane Binding of R1-3, R10-12 and the CT Domain

The data in Example 1 showed unequivocal evidence that R1-3, R10-12 and CT localize to the sarcolemma on their own. Two mechanisms can result in membrane localization: (A) direct binding to the membrane lipid bilayer via S-palmitoylation and (B) through interaction with other transmembrane proteins (e.g. the binding of the dystrophin CR-domain to β-dystroglycan). S-palmitoylation-mediated mechanism has been shown for other spectrin super-family proteins such as β-spectrin (Das, A. K. et al., J. Biol. Chem. 272, 11021-11025 (1997); Mariani et al., J. Biol. Chem. 268, 12996-13001 (1993)). Specifically, S-palmitoylation involves the addition of palmitate (a 16-carbon saturated fatty acid) to the cysteine residues of the target proteins through a reversible thioester linkage during the process of posttranslational modification (Linder, M. E. et al., Nat. Rev. Mol. Cell. Biol. 8, 74-84 (2007)). Insertion of palmitate to the lipid bilayer brings the target proteins to the plasma membrane.

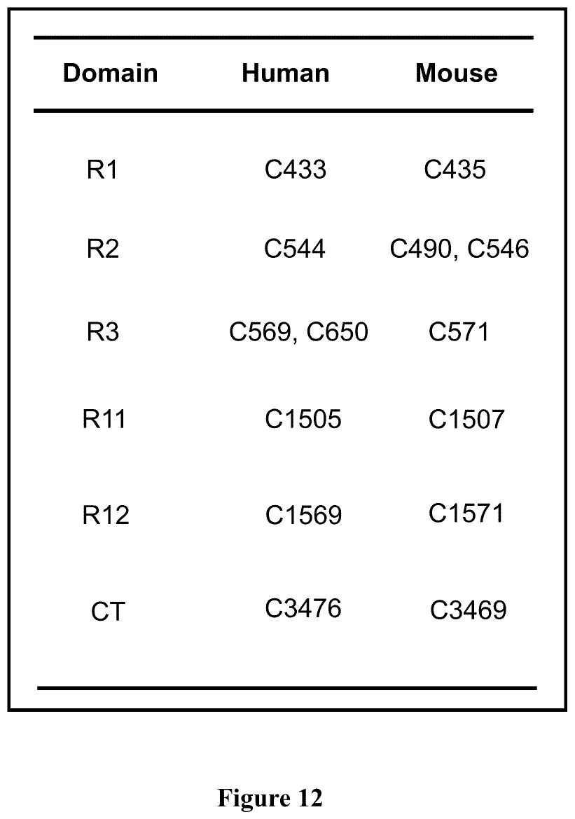

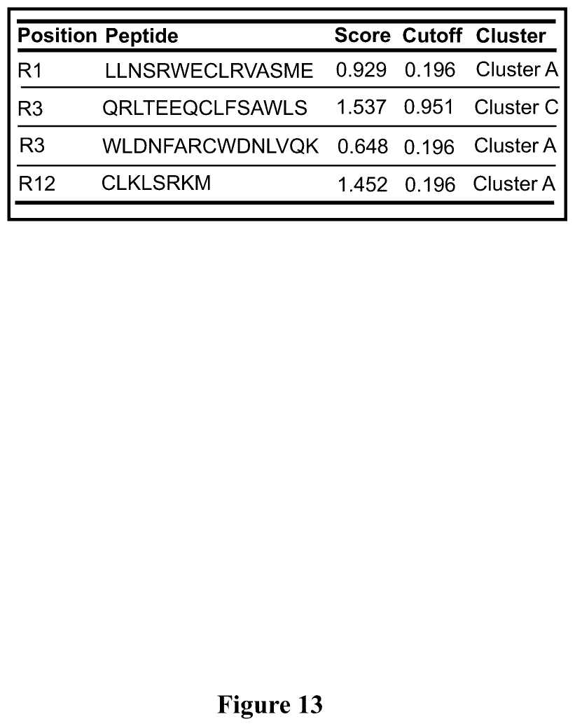

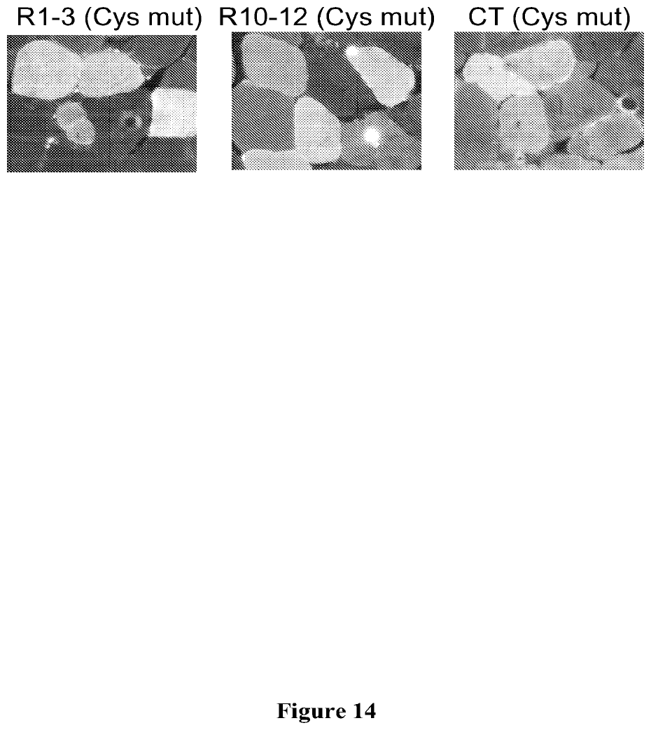

To distinguish these two potential mechanisms (direct binding via S-palmitoylation and indirect membrane binding via other membrane proteins), we examined the cysteine residues in new MBDs, and found that cysteine residues are very conserved in dystrophin R1-3, R10-12 and CT between human and mouse dystrophin (FIG. 12), indicating that cysteine residues can have an important role in the dystrophin function. In silico screening of palmitoylated sites with the CSS-Palm 2.0 program, a software for prediction of palmitoylated sites (Oku, S. et al., J. Biol. Chem. (2013); Ren, J. et al., Protein Eng Des Sel 21, 639-644 (2008)), successfully identified some palmitoylated sites in R1-3 and R10-12 (FIG. 13). Then we carried out a pilot study in which we mutated all cysteine residues in R1-3, R10-12 and the CT domain to serine (FIG. 14). Cysteine-to-serine mutation has been used by others to abolish S-palmitoylation (Topinka, J. R., et al., Neuron 20, 125-134 (1998); Yanai, A. et al. Nat. Neurosci. 9, 824-831 (2006)). We hypothesized that if S-palmitoylation mediated mechanism is responsible for sarcolemma anchoring of R1-3, R10-12 and the CT domain, cysteine-to-serine mutation should abolish S-palmitoylation and result in cytosolic location of R1-3, R10-12 and the CT domain. We made AAV vectors to express cysteine-to-serine mutated R1-3, R10-12 and the CT domain GFP fusion proteins. Following intramuscular injection to the muscle of mdx mice, we only detected cytosolic GFP signal (FIG. 14). This is in sharp contrast to what we see in FIG. 6. These results strongly suggest that S-palmitoylation is likely the predominant molecular mechanism for membrane localization of R1-3, R10-12 and the CT domain.

There are a total of four cysteine residues in R1-3, two in R10-12, and one in the CT domain. There are located in R1 (C433), R2 (C544), R3 (C569 and C650), R11 (C1505), R12 (C1569) and CT (C3476) (FIG. 12). In our preliminary study (FIG. 14), we found that mutation of all cysteine residues in each fragment abolished sarcolemmal binding.

### Example 3. Further Identification of Protein Binding Partners, Membrane Binding Motifs (MBM), Membrane Binding Repeats, and Membrane Binding Sub-Domains

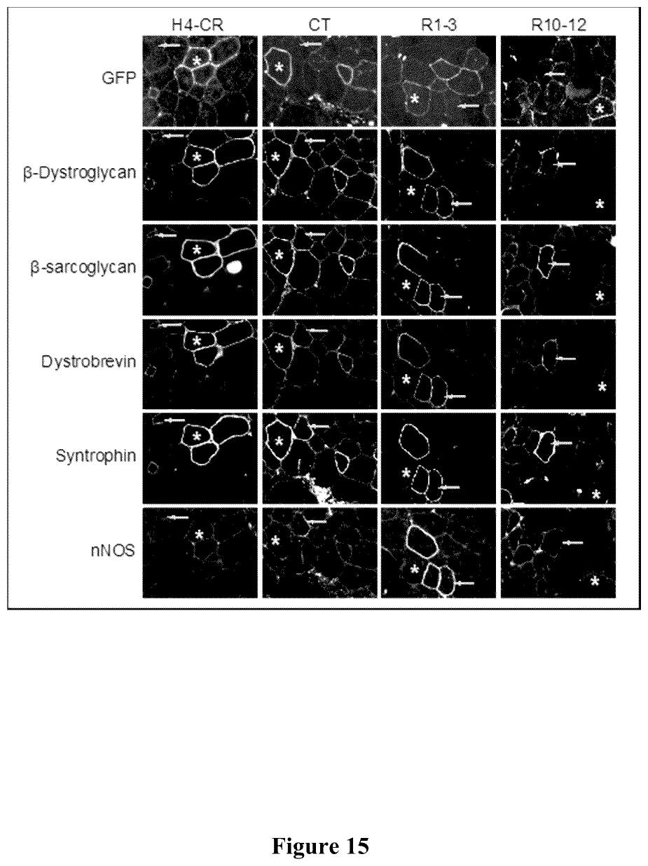

As the first step to identify protein partners of our newly discovered MBDs, we performed immunofluorescence staining using antibodies against several DGC components. These included β-dystroglycan, β-sarcoglycan, dystrobrevin, syntrophin and nNOS. We also included H4-CR.GFP as a control. We have previously shown that nNOS-binding requires R16/17, (Lai, Y. et al., J. Clin. Invest. 119, 624-635 (2009)) or an nNOS binding domain of R16/17 (Lai, Y., et al., Proc. Natl. Acad. Sci. USA 110, 525-530 (2013). As a consequence, none of the MBDs was able to restore sarcolemmal nNOS expression. Previous studies suggest that the interaction of the CR domain with β-dystroglycan is sufficient for restoration of the DGC components (Crawford, G. E. et al., J. Cell Biol. 150, 1399-1410 (2000); Yue, Y. et al., Mol Ther 14, 79-87 (2006)). As expected, H4-CR restored all DGC components. We also found that R1-3 and R10-12 did not interact with the DGC components. The CT domain by itself is associated with all the DGC components at the muscle membrane (FIG. 15).

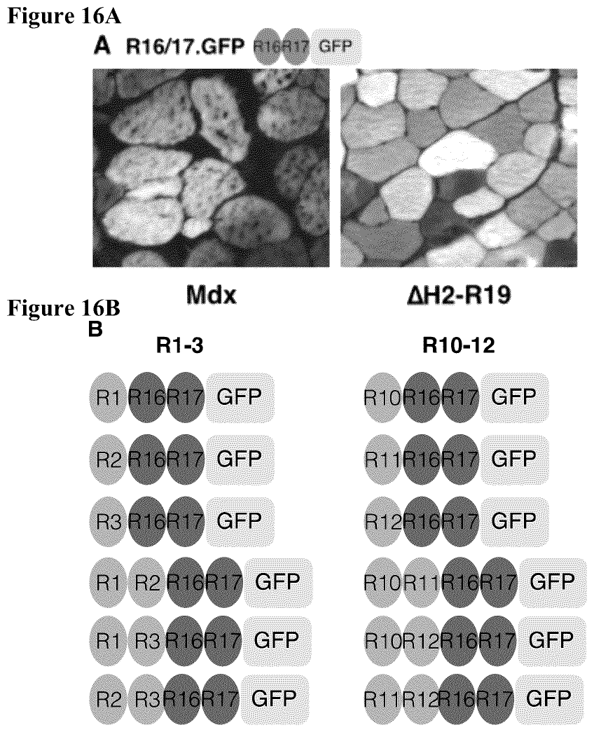

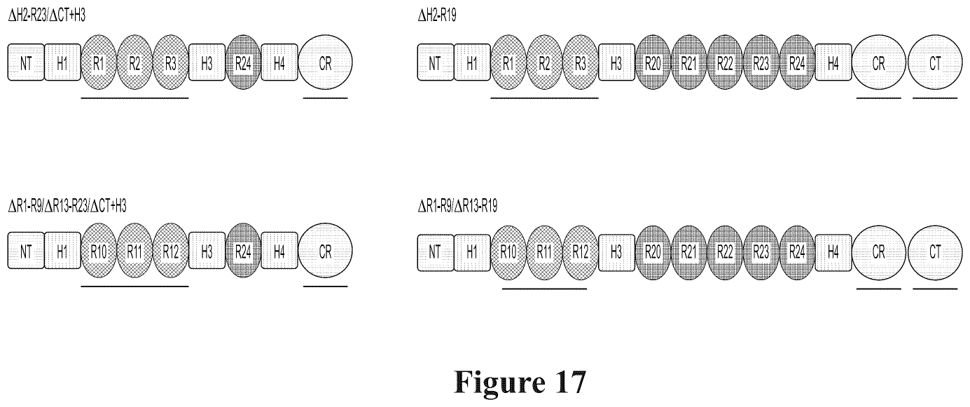

A typical feature of dystrophin membrane binding is that dystrophin MBDs are confined to two regions. Two MBDs R1-3 and R10-12 are located at the mid-rod domain, while the other two MBDs CR and CT are at the C-terminal part of dystrophin (FIG. 16). Through our preliminary data, both C-terminal MBDs (cMBDs), CR and CT, are associated with the DGC, while both rod MBDs (rMBDs), R1-3 and R10-12, are not co-localized with the components of the DGC (FIG. 17), suggesting that rMBDs and cMBDs have different functional roles. Dystrophin stabilizes and strengthens the sarcolemma by two different mechanisms: the axis from the ECM to intracellular cytoskeleton, and the membrane association from newly identified MBDs. Membrane binding of the CR domain establishes the axis from the ECM to intracellular cytoskeleton, and, in certain contexts and embodiments, the CR domain is involved in dystrophin function (Rafael, J. A. et al., J. Cell Biol. 134, 93-102 (1996)). In vitro studies have indicated that membrane binding of rMBDs is also important for membrane stability (Sarkis, J. et al., FASEB J. 27, 359-367 (2013); Sarkis, J. et al., J. Biol. Chem. (2011)). Both rMBDs are in close proximity to the muscle membrane and actin cytoskeleton. R1-3, is near the N-terminus of dystrophin, which interacts with F-actin, while R10-12 overlaps with the actin-binding domain R11-15 (FIG. 3). Simultaneous binding of R11-15 to phospholipid monolayer and F-actin considerably contributes to the stiffness and stability of the lipid monolayer (Sarkis, J. et al., FASEB J. 27, 359-367 (2013); Sarkis, J. et al., J. Biol. Chem. (2011)). So it is highly likely that the functional role of R1-3 and R10-12 is to tether actin cytoskeleton to the muscle membrane, and thereby strengthen the muscle membrane.

We will generate micro- and mini-dystrophin AAV vectors. Membrane binding of the rMBDs in truncated dystrophins will be disrupted either by cysteine mutations or by incorporating cytosolic rod domains of dystrophin. We will deliver AAV vectors to the tibialis anterior (TA) muscle of Cmah/mdx mice, examine membrane integrity by Evans blue dye uptake, and evaluate TA contractile properties and muscle histopathology. Also we will compare the function of two rMBDs: R1-3 and R101-2, in the context of truncated dystrophins to determine whether two rMBDs have equivalent function.

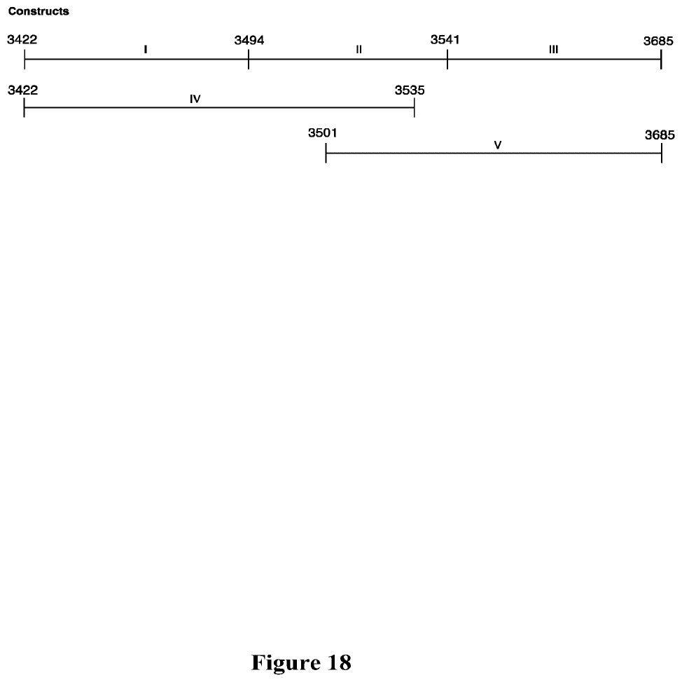

We will use two well-characterized micro- and mini-dystrophin genes as the backbones. The ΔR4-R23/ΔCT microgene and The ΔH2-R19 mini-gene have been shown to improve muscle function and correct dystrophic pathology in the dystrophic animal models (Harper, S. Q. et al., Nat. Med. 8, 253-261 (2002); Liu, M. et al., Mol Ther 11, 245-256 (2005); Lai, Y. et al., Nat. Biotechnol. 23, 1435-1439 (2005)). Both truncated dystrophins contain one rMBD, R1-3, and one cMBD, the CR domain. The ΔH2-R19 mini-dystrophin also carries another cMBD: the CT domain (FIG. 18). We will make three forms of constructs for the microgene including (1) original R1-3, (2) cysteine-mutated R1-3 and (3) replacement of R1-3 by R4-6. We will also make a similar set of constructs for the ΔH2-R19 minigene. Cysteine mutation or replacement with R4-6 will abolish the membrane binding of the rMBD, R1-3. Therefore, in the resulting truncated dystrophins, only the function of the axis from the ECM to cytoskeleton is maintained, and membrane binding from the rod domain is eliminated (FIG. 18).

Experimental mice and gene delivery. We will use Cmah/mdx double knock out mice, which have a more severe phenotype and shorter life span than mdx mice (Chandrasekharan, K. et al., Sci Transl Med 2, 42ra54 (2010)). Microgenes will be delivered to the TA muscle of Cmah/mdx mice by the single AAV vectors, while mini-dystrophins will be delivered by over-lapping AAV vectors as reported before (Odom, G. L. et al., Mol Ther 19, 36-45 (2011)). Function of truncated dystrophins and their cysteine mutants will be determined and compared. We will investigate membrane integrity by Evans blue dye uptake, measure muscle force generation and the resistance to eccentric contraction, and examine muscle histopathology, including central nucleation, myofiber size, cross section area, fibrosis and inflammation infiltration, as our published protocols (Lai, Y. et al., J. Clin. Invest. 119, 624-635 (2009); Lai, Y. et al., Nat. Biotechnol. 23, 1435-1439 (2005); Lai, Y. et al., Hum. Mol. Genet. 23, 3189-3199 (2014)). The experiments outlined above will determine whether membrane binding of R1-3 is important for dystrophin function. To investigate the functional role of another rMBD, R10-12, we will compare it to R1-3 in the context of truncated dystrophins.

Both rMBDs R1-3 and R10-12 have lipid-binding properties, and are in close proximity to the actin-binding domains. However, there are some different aspects between R1-3 and R10-12. First, the rMBD R1-3 is located at the beginning of the rod domain, while the rMBD R10-12 is in the middle of the rod domain. Second, R1-3 is exclusively located at the muscle membrane, while R10-12 is found at both the muscle membrane and cytosol (FIGS. 8 and 10). Third, all therapeutically effective truncated dystrophins only carry a partial or complete R1-3 but not R10-12. It remains unclear whether the difference between R1-3 and R10-12 represents different functional roles.

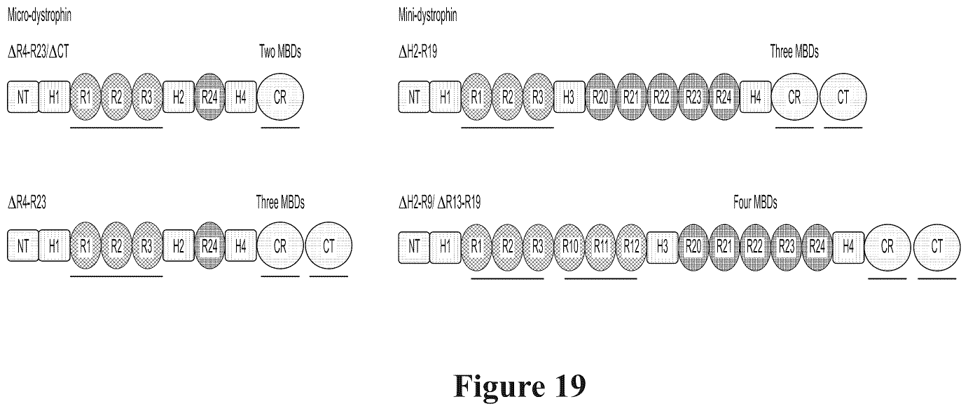

We choose ΔH2-R23/ΔCT+H3 and ΔH2-R19 micro- and mini-gene as the backbones (FIG. 19). ΔH2-R23/ΔCT+H3 is an enhanced version of ΔR4-R23/ΔCT, in which H2 was replaced with H3 (Banks, G. B. et al., PLoS Genet. 6, e1000958 (2010)). R1-3 in ΔH2-R23/ΔCT+H3 will be replaced with R10-12 to generate ΔR1-R9/ΔR13-R23/ΔCT+H3. In ΔH2-R19, we will replace R1-3 with R10-12 to generate ΔR1-R9/ΔR13-R19 mini-dystrophin. To have a fair comparison, the other components of truncated dystrophins are the same (FIG. 19).

We will use AAV gene transfer to express ΔH2-R23/ΔCT+H3, ΔR1-R9/AR13-R23/ΔCT+H3, ΔH2-R19 and ΔR1-R9/AR13-R19 in the TA muscles of Cmah/mdx mice. The ability of the truncated dystrophins to generate muscle force, maintain membrane integrity and improve histopathology of the dystrophic muscle will be measured as outlined above. These studies will tell us whether R1-3 and R10-12 have equivalent function in micro- and mini-dystrophins.

Dystrophin CR domain not only anchor to β-dystroglycan to form the axis from the ECM to intracellular cytoskeleton, but can assemble the components of DGC at the muscle membrane. Dystrophin deficiency disassembles the DGC components at the muscle membrane. Hence, restoration of the DGC components to the sarcolemma is one criterion for therapeutic outcome of truncated dystrophins.

The non-muscle dystrophin isoform Dp116 contains both cMBDs (CR and CT domain), but is deficient of both rMBDs and actin-binding domains. So Dp116 is unable to interact with F-actin. Due to the presence of both cMBDs, it can restore the DGC. Obviously, Dp116 maintains the DGC function, and loses the mechanical function to connect the ECM and cytoskeleton. In the transgenic mice expressing Dp116, dystrophic histopathology and mechanical function of the muscle were not improved. But restoration of the DGC by Dp116 is found to be crucial for growth and maintenance of muscle mass when Dp116 is expressed in the muscle of dystrophin/utrophin double knockout mice (u-dko) (Judge, L. M. et al., J. Cell Sci. 119, 1537-1546 (2006); Judge, L. M. et al., Hum. Mol. Genet. 20, 4978-4990 (2011)). These studies suggest that the mechanical function of the CR domain to connect the ECM with cytoskeleton is important for preventing dystrophic pathology, while restoration of the DGC by the CR domain is critical for muscle mass.

Truncated dystrophins without the CR domain cannot prevent dystrophic pathology, despite the presence of the other three MBDs, suggesting that the CT domain cannot compensate for mechanical function of the CR domain. Through our preliminary data, we found that either CR or CT domain alone can restore the DGC components at the muscle membrane (FIG. 17). We will determine if the CT domain can compensate for the CR domain in terms of the function in muscle mass.

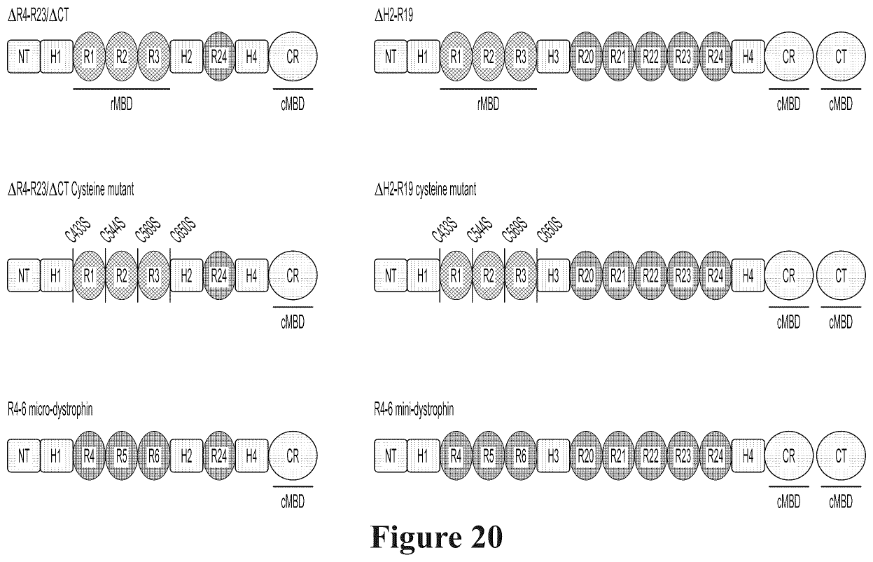

We will examine the function of the CT domain in the context of micro-dystrophins. We will use ΔR4-R23/ΔCT microgene as the backbone, and replace the CR domain with the CT domain (FIG. 20).

Experimental mice and gene delivery. We will deliver AAV.ΔR4-R23/ΔCR and AAV.ΔR4-R23/ΔCT microgenes to utrophin/dystrophin double knock-out (u-dko) mice. Since u-dko mice have a short life span, we will perform systemic delivery of AAV viruses to neonatal u-dko mice.

Outcome measurement. Two months following virus injection, the body weight of u-dko mice and muscle mass of TA and Gastro muscles will be recorded. The DGC components will be evaluated by immunostaining and western blot. Contractile properties of TA muscle will be measured.

Both cMBDs, the CR and CT domain, are located at the C-terminal end of dystrophin and can restore the DGC. In certain contexts and embodiments, CR domain is involved in dystrophin function. However, the functional significance of the CT domain is contradictory. Although CT deletion has negligible consequences in transgenic mdx mice (Rafael, J. A. et al., J. Cell Biol. 134, 93-102 (1996)), in human patients, partial or complete CT deletion can cause severe DMD phenotype (Suminaga, R. et al., Pediatr Res 56, 739-743 (2004); Prior, T. W. et al., Am. J. Hum. Genet. 57, 22-33 (1995)), indicating that CT can have important functional roles in human. In this aim, we will address a specific functional role of the CT domain in muscle mass, which will gain more insight into the function of the CT domain.

Despite the identification of R1-3, R10-12 and CT as the new MBDs of dystrophin, it is unclear whether these domains are the smallest region required for membrane binding. In spectrin, lipid-binding motif and ankyrin-binding domain have been mapped to repeats 14 and 15 of 0-spectrin (Ipsaro, J. J. et al., Blood 113, 5385-5393 (2009); Ipsaro, J. J. et al., Blood 115, 4093-4101 (2010); Bok, E. et al., Cell Biol Int 31, 1482-1494 (2007)). These results tremendously promote the efforts to solve the structure of repeats 14 and 15 of β-spectrin, which provides the structural and molecular perspective for the interactions of β-spectrin repeats 14 and 15 with lipids and ankyrin (Ipsaro, J. J. et al., Blood 113, 5385-5393 (2009); Ipsaro, J. J et al., Blood 115, 4093-4101 (2010)). We expect that mapping membrane-binding motifs in dystrophin R1-3, R10-12 and CT should be helpful for the future studies to reveal the structure of dystrophin MBDs, and facilitate our understanding of molecular basis of dystrophin membrane binding.

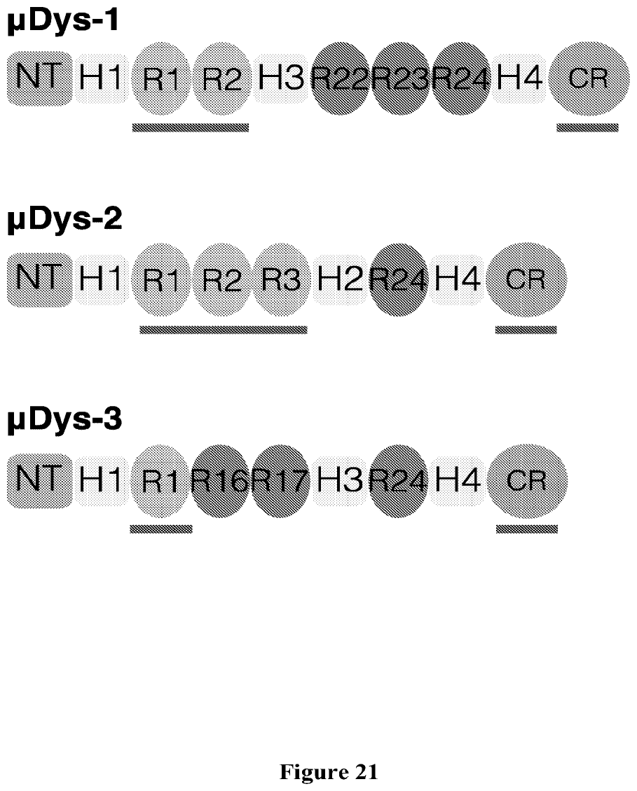

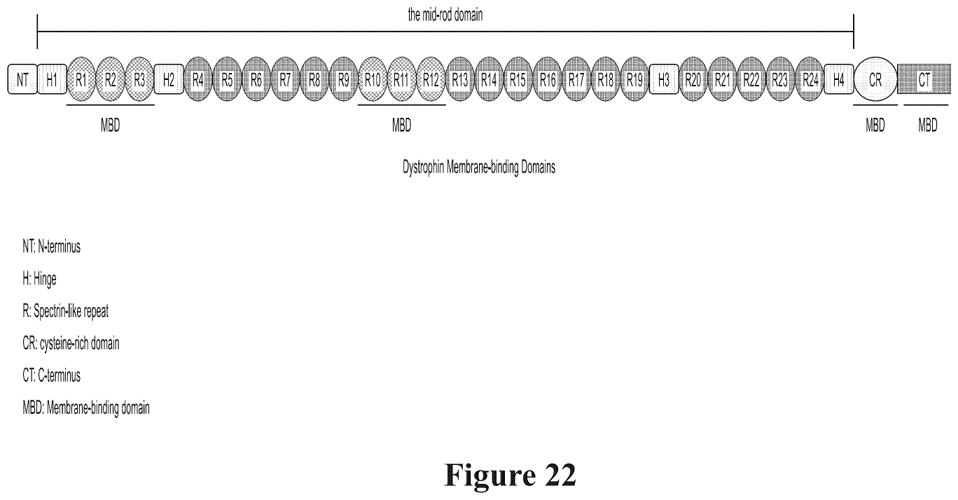

To date, there exist three functional micro-dystrophins tested in canine dystrophic models and the clinical trial. Only ΔR4-R23/ΔCT micro-dystrophin contains a complete region of R1-3, while ΔR2-R15/ΔR18-R23/ΔCT (Lai, Y. et al., J. Clin. Invest. 119, 624-635 (2009)) and Δ3900 (Wang, B. et al., Proc. Natl. Acad. Sci. USA 97, 13714-13719 (2000)) micro-dystrophin carry only R1 or R1-2, respectively (FIG. 21). But muscle force comparison revealed that there is no apparent difference regarding muscle force improvement between ΔR4-R23/ΔCT and ΔR2-R15/ΔR18-R23/ΔCT, suggesting that a partial region of R1-3 possibly maintains the ability of membrane binding. Mapping membrane-binding motifs in R1-3 will help clarify this issue.

Identification of membrane-binding motifs in R1-3, R10-12, and CT will be important for the development of DMD gene therapy. Given the packaging limit of AAV vectors, the main focus of engineering truncated dystrophins will be maximizing dystrophin function in a minimal sequence. Hence, shortening dystrophin MBDs will be useful for DMD gene therapy.

Both R1-3 and R10-12 are composed of three spectrin-like repeats. First we ask whether the single repeat or bi-repeats of R1-3 and R10-12 maintain the ability of membrane binding. To address this issue, we will split R1-3 and R10-12 into smaller individual repeats, and use AAV.R16/17.GFP construct as the backbone, since our previous study has shown that R16/17.GFP is expressed in the cytosol of myofibers, and R16/17 are an important component of the microgene (Lai, Y. et al., Proc. Natl. Acad. Sci. USA 110, 525-530 (2013)). And we will fuse R1, R2, R3, R1-2, R2-3, R1,3 or R10, R11, R12, R10-11, R11-12, R10,12 to R16/17.GFP (FIG. 16), and exploit AAV gene transfer to express the GFP fusion proteins in the muscle of mdx 4cv mice. Membrane binding of the GFP fusion proteins will be determined by the GFP signal and immunostaining with the epitope-specific antibodies. If the single repeat or bi-repeats maintain the membrane-binding ability, they will target the R16/17.GFP to the muscle membrane. The information gathered from these studies will help us determine which repeats in R1-3 and R10-12 have the ability of membrane binding, and will clarify whether the partial R1-3 in some micro-dystrophins conserves membrane binding.

Those repeats with the ability of membrane binding are named as membrane-binding repeats. Each spectrin-like repeat consists of three α-helices. Next, we will proceed to narrow down the membrane-binding motifs to the helices of the membrane-binding repeats. In our previous study, we successfully determined a 10-amino-acid nNOS-binding motif in the first helix of R17, and also found that two upstream and downstream helices that flank nNOS-binding motif are also required for nNOS binding since the flanking helices frame the nNOS-binding motif and make it accessible to nNOS binding (Lai, Y. et al., Proc. Natl. Acad. Sci. USA 110, 525-530 (2013)). Here, we will use the same strategy to decide the membrane-binding motifs in membrane-binding repeats.

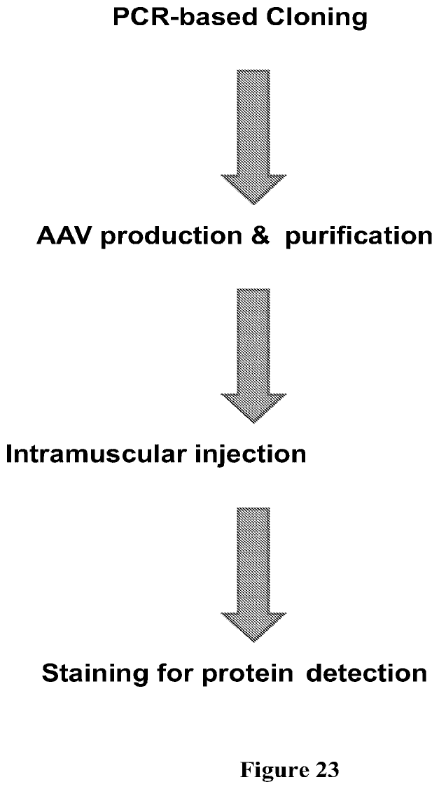

We will choose AAV constructs that contain membrane-binding repeats as the backbones (FIGS. 19, 20, and 21). Like our previous study, we will replace the individual helix in the membrane-binding repeats with the corresponding helix from R16 to determine which helices in the membrane-binding repeats are involved in membrane binding. For the helices that are involved in membrane binding, we will split each helix into 4-5 parts, each part containing 9-10 amino acids, and replace each part with the corresponding region from R16. Then we will express these mutants by AAV gene transfer in the TA muscle of mdx 4cv mice, and determine the membrane binding of these mutants by the GFP signal and immunostaining. An example of a methodology used to test various constructs is shown (FIG. 23). These studies will further narrow down the membrane-binding motifs in the membrane-binding repeats of R1-3 and R10-12.

We will use the deletion strategy to identify the membrane-binding motif in the CT domain. The construct AAV.CT.GFP shown in FIG. 5 will be used as the backbone. Different partial deletions of the CT domain will be introduced to AAV.CT.GFP construct as outlined in FIG. 18. We will use AAV gene transfer to deliver these constructs to the TA muscle of mdx 4cv mice. The membrane localization of the GFP fusion proteins will be determined by the GFP signal. If we decide which part of the CT domain is responsible for membrane binding, we will split this part into three smaller motifs, and narrow down the membrane-binding region to the smallest motif.

### Example 4. Construction of New Dystrophin MBDs into Micro- and Mini-Dystrophin Synthetic Genes and Insertion of Same into AAV Vectors

In vitro studies have shown that membrane association from newly discovered MBDs is important for dystrophin function (Sarkis, J. et al., FASEB J. 27, 359-367 (2013); Sarkis, J. et al., J. Biol. Chem. (2011)). However, currently available micro-dystrophins contain two MBDs: partial or complete R1-3 and the CR domain, while mini-dystrophins ΔH2-R19 and ΔH2-R15 carry three MBDs: R1-3, CR and CT, suggesting that the membrane-binding ability of truncated dystrophins is compromised. Here, we will generate new dystrophin AAV vectors by adding more MBDs.

For initial testing, we will use the ΔR4-R23/ΔCT microgene as the backbone, since ΔR4-R23/ΔCT microgene is the only microgene containing the complete MBD, R1-3 (FIG. 21). The micro-dystrophins are packaged by the single AAV vector, which has a packaging limit of about 4.9 kb. The original size of ΔR4-R23/ΔCT AAV vector is about 4.8 kb, including 3.6 kb micro-dystrophin cDNA, a 523 bp CMV promoter, a 206 bp SV40 PolyA site, 0.3 kb AAV ITRs, and other sequences for 5′ and 3′ untranslated regions (UTR) and multiple cloning sites. We will free up space for an additional MBD by shortening transcription regulation elements and sequences for UTRs and cloning sites. A shortened muscle-specific promoter and a synthetic PolyA site (49 bp) (Levitt, N. et al., Genes Dev. 3, 1019-1025 (1989)) will replace the CMV promoter and SV40 PolyA site. Also the sequences for UTRs and the cloning sites will be shortened by engineering the shorter UTRs, and including the cloning sites into the UTRs. To make the total size of micro-dystrophin AAV vector about 4.9 kb, these changes allow us to add >700 bp more bps in the ΔR4-R23/ΔCT micro-dystrophin. Since each spectrin-like repeat is about 330 bps and the CT domain is about 792 bps, the spared space can hold two more repeats or one more repeat and half of the CT domain or the whole CT domain. Since the shortest membrane-binding regions are first being identified, only the CT domain can be added to microgenes. For the first test, we will add the CT domain into ΔR4-R23/ΔCT micro-dystrophin without affecting the packaging efficiency of AAV vectors. So the resultant microgene ΔR4-R23 contains three MBDs (FIG. 19).

ΔH2-R19 mini-dystrophin contains three MBDs: R1-3, CR and CT domain. It can restore full muscle force but only partially recover heart hemodynamic function (Bostick, B. et al., Mol Ther 17, 253-261 (2009)). So we will use ΔH2-R19 mini-dystrophin as the backbone, and engineer R10-12 into ΔH2-R19 mini-dystrophin to make a new mini-dystrophin with four MBDs (FIG. 19).

These two constructs are two examples for how we will engineer new dystrophin AAV vectors by adding more MBDs into dystrophin AAV vectors. The list of micro- and mini-dystrophin AAV vectors can be expanded once the smallest membrane-binding region is identified from the preceding studies. For example, if rMBDs, R1-3 and R10-12, could be reduced to the single repeat, we can make the micro-dystrophin with two rMBDs and one cMBD, the CR domain. If one half of the CT domain can be trimmed, we could even make new micro-dystrophin AAV vector containing all four MBDs. If the membrane-binding motifs can be reduced to the helices, we can generate a hybrid repeat. For example, R16/17 are essential for nNOS binding. The first helix of R16 can be replaced without affecting nNOS binding. We can engineer the membrane-binding motif from R1-3 or R10-12 into the first helix of R16 to generate a hybrid repeat with two functions.

To examine therapeutic efficacy of new micro- and mini-dystrophins in murine and canine dystrophic models, we will deliver new dystrophin AAV vectors to Cmah/mdx mice and DMD dogs and examine therapeutic efficacy of these new dystrophin AAV vectors. All new dystrophin AAV vectors will be tested in Cmah/mdx first. Contractile properties of TA muscle, ECG and hemodynamic function, membrane integrity and muscle histopathology will be examined as outlined above. From the functional results, one best microgene and one best minigene will be selected for further testing in DMD dogs.

The therapeutic efficacy of new micro- and mini-dystrophins will be tested in DMD dogs. A series of functional studies in canine dystrophic models, including measurements of single muscle force, cardiac function and blood flow (Yang, H. T. et al. PLoS One 7, e44438 (2012); Fine, D. M. et al., Neuromuscul Disord 21, 453-461 (2011)) can be performed. Micro- and mini-dystrophin AAV vectors will be delivered to 5-6 DMD dogs, respectively. For virus injection in DMD dogs, a transient immunosuppression protocol will be administered. And AAV vectors will be injected to the Extensor Carpi Ulnaris (ECU) muscle of DMD dogs by intramuscular (IM) injection. After five to six months, force generation and the resistance to eccentric contraction of ECU muscle will be evaluated (Yang, H. T. et al., PLoS One 7, e44438 (2012); Shin, J. H. et al., Mol Ther 21, 750-757 (2013)). Histopathology will be investigated as proposed in the mouse studies.

Despite the role of cysteine residues in membrane binding of R1-3, R10-12 and the CT domain, the shortest membrane-binding region is still unknown. In this aim, we will identify membrane-binding motifs by AAV gene transfer. Hence, the membrane-binding motifs derived from this study will be highly relevant to DMD gene therapy. A previous study has shown that the single repeat R2 has lipid-binding ability (Le Rumeur, E. et al. Biochim. Biophys. Acta 1768, 648-654 (2007)) suggesting that the individual repeat from R1-3 can bind to the muscle membrane. So it is likely that the R1-3 membrane-binding region can be shortened.

Currently available truncated dystrophins are not fully functional. We will generate a series of new dystrophin AAV vectors that contain more MBDs to improve their therapeutic effects. First we will examine therapeutic effects of new dystrophin AAV vectors in the mouse model. Only after we confirm that new dystrophin AAV vectors perform better than original dystrophin AAV vectors, we will proceed to test the best candidates in the canine dystrophic model.

### Example 5. Restoration of Sarcolemmal nNOS in Mdx Mice by Dystrophin Spectrin-Like Repeats 16 and 17 and Syntrophin PDZ Fusion Protein

Duchenne Muscular Dystrophy (DMD) is a genetic disorder that affects sarcolemmal localization of neuronal nitric oxide synthase (nNOS). Sarcolemmal nNOS is required for muscle cells to function properly. In DMD patients, a deficiency in the dystrophin protein leads to a reduction in sarcolemmal nNOS and syntrophin. From a previous study (Lai, Yi, et al. Journal of Clinical Investigation (2009): 624-35), recruitment of sarcolemmal nNOS is dependent on dystrophin spectrin-like repeats 16 and 17 (R16/17) and syntrophin PDZ domain.

Muscle wasting diseases such as Duchenne muscular dystrophy (DMD) affect sarcolemmal localization of neuronal nitric oxide synthase (nNOS). Sarcolemmal nNOS is required for muscle cells to function properly. Sarcolemmal localization of nNOS is dependent on its simultaneous binding to dystrophin spectrin-like repeats 16 and 17 (R16/17) and syntrophin PDZ domain. DMD is characterized by a deficiency in dystrophin. In DMD, loss of dystrophin leads to the reduction or loss of syntrophin at the sarcolemma, which further results in the loss of sarcolemmal nNOS. Loss of sarcolemmal neuronal nitric oxide synthase (nNOS) is a salient pathogenic feature in muscle wasting conditions/diseases such as age-related muscle atrophy, cancer cachexia, Duchenne muscular dystrophy (DMD) and many other neuromuscular disorders.

In a previous study, dystrophin R16/17 was expressed in the muscle of a truncated dystrophin transgenic mouse, where syntrophin is present at the membrane. The results showed that sarcolemmal nNOS was recovered successfully, indicating that dystrophin R16/17 and syntrophin PDZ are required for sarcolemmal nNOS.

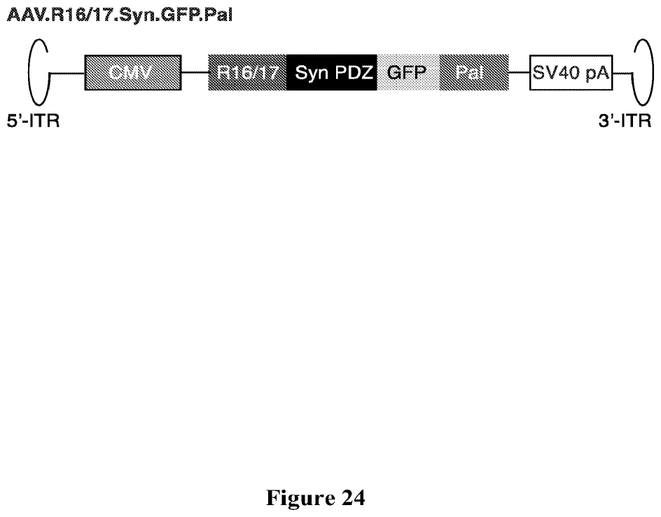

In this study, we engineered an adeno-associated virus (AAV) vector that can express a dystrophin R16/17-syntrophin PDZ fusion protein. We tested whether the expression of the fusion protein restored sarcolemmal nNOS in the muscle of mdx mice, the DMD mouse model (FIG. 23). PCR-based cloning was used to clone syntrophin PDZ into the AAV.R16/17.GFP.Pal backbone to produce AAV.R16/17.Syn.GFP.Pal construct (FIG. 24). In the vector, a hinge region (GGSG) was inserted between R16/17 and syn PDZ. GFP is a tag that helps detect the R16/17.Syn protein. Pal is the signal for membrane targeting. The AAV plasmid DNA was amplified to produce large amounts of DNA for virus production. We then performed a local injection of the virus into six, −3.5 month old mdx mice. Each mouse received 1.4*10¹² viral genome particles (vg) into the tibialis anterior and 2.2*10¹² vg into the gastrocnemius muscles. Three weeks later, we harvested the muscle tissues. First, we confirmed the expression of the R16/17-syntrophin PDZ fusion protein in the muscle by fluorescence microscopy for the GFP signal. Then we performed immunostaining and nNOS activity staining to examine if the expression of R16/17-syntrophin PDZ fusion protein can restore sarcolemmal nNOS.

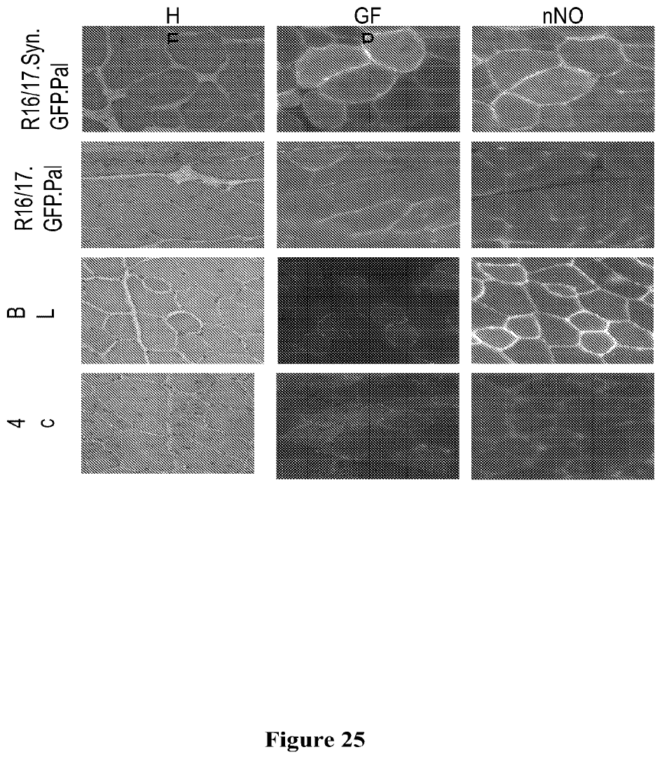

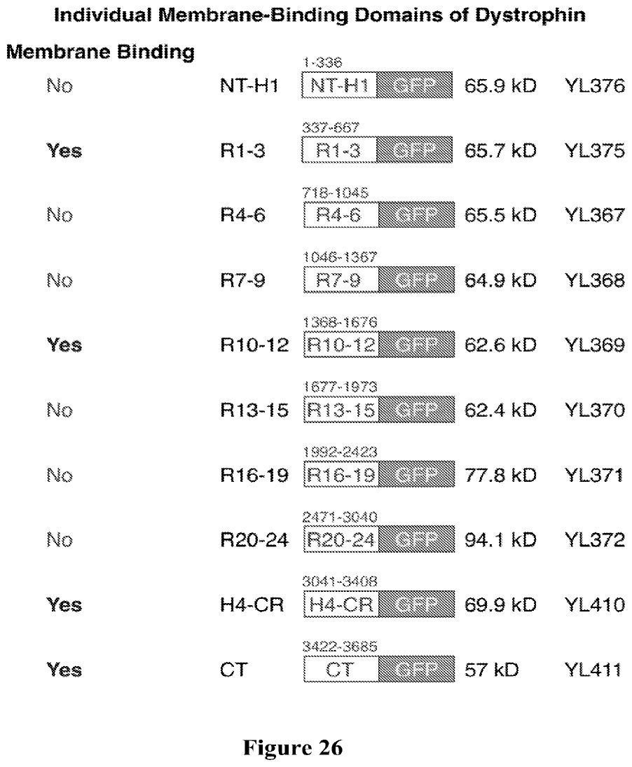

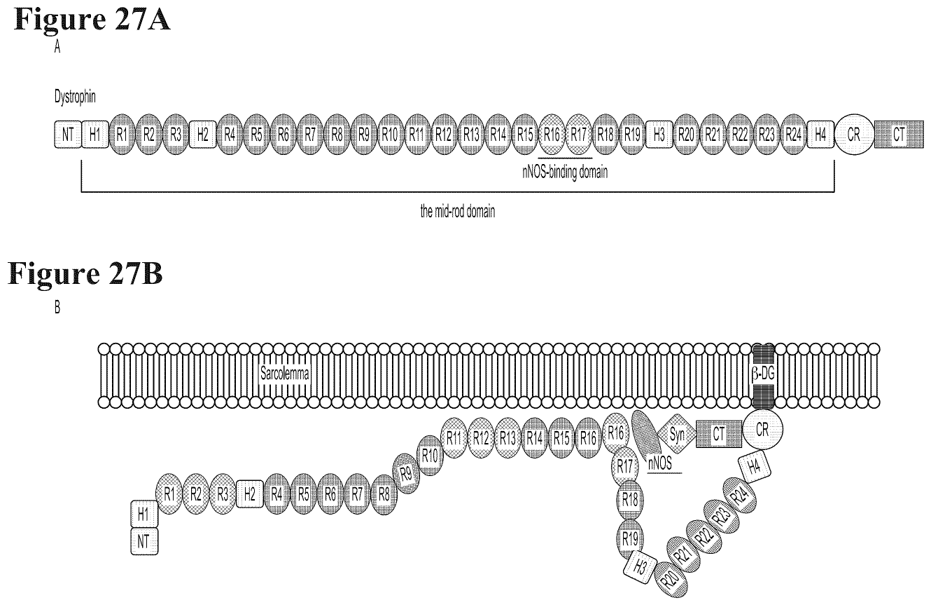

Our results show that sarcolemmal nNOS was recovered successfully with the use of R16/17-syntrophin PDZ fusion protein (FIG. 25). Further testing will be done to examine the therapeutic effects of restoring sarcolemmal nNOS. Restoration of sarcolemmal nNOS has therapeutic use for multiple neuromuscular disorders, such as DMD, and other muscle wasting conditions such as age/inactivity-related muscle atrophy and cancer cachexia.

DMD is a disorder that is characterized by degeneration and regeneration of muscle tissues and premature death most commonly due to cardiac or respiratory failure. In patients suffering from DMD, sarcolemmal nNOS is either reduced or completely lost. Sarcolemmal nNOS plays a crucial role in the upkeep of muscle tissues.

The results from this project show that it is possible to introduce sarcolemmal localization of nNOS in mdx mice with the use of a viral vector. Our next step is to see whether or not the R16/17-syntrophin PDZ fusion protein can recruit nNOS in DBA/mdx mice, a more severe phenotype mouse model of DMD.

Example 6. Description of Sequences Provided
in the Sequence Listing
A description of sequences provided herewith
in the electronic sequence listing file
“17UMC006_SEQ LST_TC167044_ST25.txt”
follows below.
SEQ ID NO: 1:
Full-length human dystrophin protein sequence
SEQ ID NO: 2:
Full-length dystrophin coding region
SEQ ID NO: 3:
.DELTA.17-48 (mini-dystrophin with 8.5 repeats
and 3 hinges) (This minigene does not carry
R16 or R17. It cannot restore nNOS)
SEQ ID NO: 4:
.DELTA.H2-R19 (mini-dystrophin with 8 repeats
and 3 hinges) (This minigene does not carry
R16 or R17. It cannot restore nNOS)
SEQ ID NO: 5:
.DELTA.H2-R17 (mini-dystrophin with 10 repeats
and 3 hinges) (This minigene does not carry
R16 or R17. It cannot restore nNOS)
SEQ ID NO: 6:
.DELTA.H2-R16 (mini-dystrophin with 11 repeats
and 3 hinges) (This minigene carries R17 but
not R16. It cannot restore nNOS)
SEQ ID NO: 7:
.DELTA.H2-R15 (mini-dystrophin with 12 repeats
and 3 hinges) (This minigene carries both R16
and R17. It can restore nNOS)
SEQ ID NO: 8:
.DELTA.H2-R15/.DELTA.R18-19 (mini-dystrophin
with 10 repeats and 3 hinges) (This minigene
carries both R16 and R17. It can restore nNOS)
SEQ ID NO: 9:
.DELTA.H2-R15/.DELTA.17-19 (mini-dystrophin
with 9 repeats and 3 hinges) (This minigene
carries R16 but not R17. It cannot restore
nNOS)
SEQ ID NO: 10:
.DELTA.H2-R15/.DELTA.C (mini-dystrophin with
12 repeats and 3 hinges, no C-terminal domain)
(This minigene carries both R16 and R17. It
can restore nNOS)
SEQ ID NO: 11:
.DELTA.R2-R15/.DELTA.H3-R23/.DELTA.C (micro-
dystrophin with 6 repeats and 2 hinges, no
C-terminal domain) (This microgene carries
both R16 and R17. It can restore nNOS)
SEQ ID NO: 12:
.DELTA.R3-R15/.DELTA.R18-23/.DELTA.C (micro-
dystrophin with 5 repeats and 2 hinges, no
C-terminal domain) (This microgene carries
both R16 and R17. It can restore nNOS)
SEQ ID NO: 13:
.DELTA.R2-R15/.DELTA.R18-23/.DELTA.C (micro-
dystrophin with 4 repeats and 2 hinges, no
C-terminal domain) (This microgene carries
both R16 and R17. It can restore nNOS)
SEQ ID NO: 14:
.DELTA.R3-R15/.DELTA.R17-23/.DELTA.C (micro-
dystrophin with 4 repeats and 2 hinges, no
C-terminal domain) (This microgene carries
R16 but not R17. It cannot restore nNOS)
SEQ ID NO: 15:
AV.CMV..DELTA.R2-15/.DELTA.R18-23/.DELTA.C
(This AAV vector contains four repeats and
two hinges. It carries both R16 and R17 and
it can restore nNOS)
SEQ ID NO: 16:
AV.CMV..DELTA.R3-15/.DELTA.R18-23/.DELTA.C
(This AAV vector contains five repeats and
two hinges. It carries both R16 and R17 and
it can restore nNOS)
SEQ ID NO: 17:
Human dystrophin domain sequence N-terminal
domain
SEQ ID NO: 18:
Hinge 1
SEQ ID NO: 19:
Repeat 1
SEQ ID NO: 20:
Repeat 2
SEQ ID NO: 21:
Repeat 3
SEQ ID NO: 22:
Hinge 1
SEQ ID NO: 23:
Repeat 4
SEQ ID NO: 24:
Repeat 5
SEQ ID NO: 25:
Repeat 6
SEQ ID NO: 26:
Repeat 7
SEQ ID NO: 27:
Repeat 8
SEQ ID NO: 28:
Repeat 9
SEQ ID NO: 29:
Repeat 10
SEQ ID NO: 30:
Repeat 11
SEQ ID NO: 31:
Repeat 12
SEQ ID NO: 32:
Repeat 13
SEQ ID NO: 33:
Repeat 14
SEQ ID NO: 34:
Repeat 15
SEQ ID NO: 35:
Repeat 16
SEQ ID NO: 36:
Repeat 17
SEQ ID NO: 37:
Repeat 18
SEQ ID NO: 38:
Repeat 19
SEQ ID NO: 39:
Hinge 3
SEQ ID NO: 40:
Repeat 20
SEQ ID NO: 41:
Repeat 21
SEQ ID NO: 42:
Repeat 22
SEQ ID NO: 43:
Repeat 23
SEQ ID NO: 44:
Repeat 24
SEQ ID NO: 45:
Hinge 4
SEQ ID NO: 46:
Cysteine-rich domain
SEQ ID NO: 47:
C-terminal domain
SEQ ID NO: 48:
Full-length canine dystrophin DNA sequence
SEQ ID NO: 49:
Full-length canine dystrophin protein sequence
SEQ ID NO: 50:
N-terminal domain from 1 aa to 252 aa; total
252 aa of full length human dystrophin protein
of 3685 aa)
SEQ ID NO: 51:
Mid-rod domain (from 253 aa to 3112 aa; total
2860 aa of full length human dystrophin protein
of 3685 aa)
SEQ ID NO: 52:
Cysteine-rich domain (from 3113 aa to 3408 aa;
total 296 aa of full length human dystrophin
protein of 3685 aa)
SEQ ID NO: 53:
C-terminal domain (from 3409 aa to 3695 aa;
total 277 aa of full length human dystrophin
protein of 3685 aa)
SEQ ID NO: 54:
LLNSRWECLRVASME
SEQ ID NO: 55:
QRLTEEQCLFSAWLS
SEQ ID NO: 56:
WLDNFARCWDNLVQK
SEQ ID NO: 57:
CLKLSRKM
R16 peptide sequence (first alpha-helix
underlined):
SEQ ID NO: 58
EISYVPSTYLTEITHVSQALLEVEQLLNAPDLCAKDFEDLFKQEESLKNI
KDSLQQSSGRIDIIHSKKTAALQSATPVERVKLQEALSQLDFQWEKVNKM
YKDRQGRFDR
first alpha-helix of R16:
SEQ ID NO: 59
PSTYLTEITHVSQALLEVEQL
(R10-R11-R12 peptide; MBM underlined):
SEQ ID NO: 60
SIQSAQETEKSLHLIQESLTFIDKQLAAYIADKVDAAQMPQEAQKIQSDL
TSHEISLEEMKKHNQGKEAAQRVLSQIDVAQKKLQDVSMKFRLFQKPANF
ELRLQESKMILDEVKMHLPALETKSVEQEVVQSQLNHCVNLYKSLSEVKS
EVEMVIKTGRQIVQKKQTENPKELDERVTALKLHYNELGAKVTERKQQLE
KCLKLSRKMRKEMNVLTEWLAATDMELTKRSAVEGMPSNLDSEVAWGKAT
QKEIEKQKVHLKSITEVGEALKTVLGKKETLVEDKLSLLNSNWIAVTSRA
EEWLNLLLE
(R1-R2-R3 peptide; MBM underlined):
SEQ ID NO: 61
SEVNLDRYQTALEEVLSWLLSAEDTLQAQGEISNDVEVVKDQFHTHEGYM
MDLTAHQGRVGNILQLGSKLIGTGKLSEDEETEVQEQMNLLNSRWECLRV
ASMEKQSNLHRVLMDLQNQKLKELNDWLTKTEERTRKMEEEPLGPDLEDL
KRQVQQHKVLQEDLEQEQVRVNSLTHMVVVVDESSGDHATAALEEQLKVL
GDRWANICRWTEDRWVLLQDILLKWQRLTEEQCLFSAWLSEKEDAVNKIH
TTGFKDQNEMLSSLQKLAVLKADLEKKKQSMGKLYSLKQDLLSTLKNKSV
TQKTEAWLDNFARCWDNLVQKLEKSTAQISQ
SEQ ID NO: 62:
GGSG
SEQ ID NO: 63:
GGGS
SEQ ID NO: 64:
GGGGS
SEQ ID NO: 65:
GSAT
SEQ ID NO: 66:
(PDZ domain of mouse syntrophin)
SEQ ID NO: 67:
(PDZ Domain of Human syntrophin)

### REFERENCES

1. Straub, V., Rafael, J. A., Chamberlain, J. S. and Campbell, K. P. (1997) Animal models for muscular dystrophy show different patterns of sarcolemmal disruption. J. Cell Biol., 139, 375-385.
2. Petrof, B. J., Shrager, J. B., Stedman, H. H., Kelly, A. M. and Sweeney, H. L. (1993) Dystrophin protects the sarcolemma from stresses developed during muscle contraction. Proc. Natl. Acad. Sci. USA, 90, 3710-3714.
3. Hoffman, E. P., Brown, R. H. J. and Kunkel, L. M. (1987) Dystrophin: the protein product of the Duchenne muscular dystrophy locus. Cell, 51, 919-928.
4. Koenig, M. and Kunkel, L. M. (1990) Detailed analysis of the repeat domain of dystrophin reveals four potential hinge segments that can confer flexibility J. Biol. Chem., 265, 4560-4566.
5. Amann, K. J., Renley, B. A. and Ervasti, J. M. (1998) A cluster of basic repeats in the dystrophin rod domain binds F-actin through an electrostatic interaction. J. Biol. Chem., 273, 28419-28423.
6. Rybakova, I. N., Amann, K. J. and Ervasti, J. M. (1996) A new model for the interaction of dystrophin with F-actin. J. Cell Biol., 135, 661-672.
7. Campbell, K. P. and Kahl, S. D. (1989) Association of dystrophin and an integral membrane glycoprotein. Nature, 338, 259-262.
8. Jung, D., Yang, B., Meyer, J., Chamberlain, J. S. and Campbell, K. P. (1995) Identification and characterization of the dystrophin anchoring site on beta-dystroglycan. J. Biol. Chem., 270, 27305-27310.
9. Huang, X., Poy, F., Zhang, R., Joachimiak, A., Sudol, M. and Eck, M. J. (2000) Structure of a WW domain containing fragment of dystrophin in complex with beta-dystroglycan. Nat. Struct. Biol., 7, 634-638.
10. Ervasti, J. M. and Campbell, K. P. (1991) Membrane organization of the dystrophin-glycoprotein complex. Cell, 66, 1121-1131.
11. Helliwell, T. R., Ellis, J. M., Mountford, R. C., Appleton, R. E. and Morris, G. E. (1992) A truncated dystrophin lacking the C-terminal domains is localized at the muscle membrane. Am. J. Hum. Genet., 50, 508-514.
12. Hoffman, E. P., Garcia, C. A., Chamberlain, J. S., Angelini, C., Lupski, J. R. and Fenwick, R. (1991) Is the carboxyl-terminus of dystrophin required for membrane association? A novel, severe case of Duchenne muscular dystrophy. Ann. Neurol., 30, 605-610.
13. Recan, D., Chafey, P., Leturcq, F., Hugnot, J. P., Vincent, N., Tome, F., Collin, H., Simon, D., Czernichow, P., Nicholson, L. V. and et, A. (1992) Are cysteine-rich and COOH-terminal domains of dystrophin critical for sarcolemmal localization? J. Clin. Invest., 89, 712-716.
14. Le Rumeur, E., Winder, S. J. and Hubert, J. F. (2010) Dystrophin: more than just the sum of its parts. Biochim. Biophys. Acta, 1804, 1713-1722.
15. Cox, G. A., Sunada, Y., Campbell, K. P. and Chamberlain, J. S. (1994) Dp71 can restore the dystrophin-associated glycoprotein complex in muscle but fails to prevent dystrophy. Nat. Genet., 8, 333-339.
16. Crawford, G. E., Faulkner, J. A., Crosbie, R. H., Campbell, K. P., Froehner, S. C. and Chamberlain, J. S. (2000) Assembly of the dystrophin-associated protein complex does not require the dystrophin COOH-terminal domain J. Cell Biol., 150, 1399-1410.
17. Rapaport, D., Greenberg, D. S., Tal, M., Yaffe, D. and Nudel, U. (1993) Dp71, the nonmuscle product of the Duchenne muscular dystrophy gene is associated with the cell membrane. FEBS Lett., 328, 197-202.
18. Judge, L. M., Haraguchiln, M. and Chamberlain, J. S. (2006) Dissecting the signaling and mechanical functions of the dystrophin-glycoprotein complex J. Cell Sci., 119, 1537-1546.
19. Rafael, J. A., Cox, G. A., Corrado, K., Jung, D. Campbell, K. P. and Chamberlain, J. S. (1996) Forced expression of dystrophin deletion constructs reveals structure-function correlations. J. Cell Biol., 134, 93-102.
20. Fritz, J. D., Danko, I., Roberds, S. L., Campbell, K. P., Latendresse, J. S. and Wolff, J. A. (1995) Expression of deletion-containing dystrophins in mdx muscle: implications for gene therapy and dystrophin function. Pediatr Res, 37, 693-700.
21. Maconochie, M. K., Simpkins, A. H., Damien, E., Coulton, G., Greenfield, A. J. and Brown, S. D. (1996) The cysteine-rich and C-terminal domains of dystrophin are not required for normal costameric localization in the mouse. Transgenic Res, 5, 123-130.
22. Gardner, K. L., Kearney, J. A., Edwards, J. D. and Rafael-Fortney, J. A. (2006) Restoration of all dystrophin protein interactions by functional domains in trans does not rescue dystrophy. Gene Ther, 13, 744-751.
23. Bamabei, M. S., Sjaastad, F. V., Townsend, D., Bedada, F. B. and Metzger, J. M. (2015) Severe dystrophic cardiomyopathy caused by the enteroviral protease 2A-mediated C-terminal dystrophin cleavage fragment. Sci Transl Med, 7, 294ra106.
24. Dunckley, M. G Wells, K. E., Piper, T. A., Wells, D. J. and Dickson, G. (1994) Independent localization of dystrophin N- and C-terminal regions to the sarcolemma of mdx mouse myofibres in vivo. J. Cell Sci., 107, 1469-1475.
25. Hir, S. A., Raguenes-Nicol, C., Paboeuf, G., Nicolas, A., Le Rumeur, E. and Vie, V. (2014) Cholesterol favors the anchorage of human dystrophin repeats 16 to 21 in membrane at physiological surface pressure. Biochim. Biophys. Acta, 1838, 1266-1273.
26. DeWolf, C., McCauley, P., Sikorski, A. F., Winlove, C. P., Bailey, A. I., Kahana, E., Pinder, J. C. and Gratzer, W. B. (1997) Interaction of dystrophin fragments with model membranes. Biophys. J., 72, 2599-2604.
27. Le Rumeur, E., Fichou, Y., Pottier, S., Gaboriau, F., Rondeau-Mouro, C., Vincent, M., Gallay, J. and Bondon, A. (2003) Interaction of dystrophin rod domain with membrane phospholipids. Evidence of a close proximity between tryptophan residues and lipids. J. Biol. Chem., 278, 5993-6001.
28. Le Rumeur, E., Pottier, S., Da Costa, G., Metzinger, L., Mouret, L., Rocher, C., Fourage, M., Rondeau-Mouro, C. and Bondon, A. (2007) Binding of the dystrophin second repeat to membrane di-oleyl phospholipids is dependent upon lipid packing. Biochim. Biophys. Acta, 1768, 648-654.
29. Legardinier, S., Hubert, J. F., Le Bihan, O., Tascon, C., Rocher, C., Raguenes-Nicol, C., Bondon, A., Hardy, S. and Le Rumeur, E. (2008) Sub-domains of the dystrophin rod domain display contrasting lipid-binding and stability properties. Biochim. Biophys. Acta, 1784, 672-682.
30. Legardinier, S., Raguenes-Nicol, C., Tascon, C., Rocher, C., Hardy, S., Hubert, J. F. and Le Rumeur, E. (2009) Mapping of the lipid-binding and stability properties of the central rod domain of human dystrophin. J. Mol. Biol., 389, 546-558.
31. Lai, Y., Zhao, J., Yue, Y. and Duan, D. (2013) alpha2 and alpha3 helices of dystrophin R16 and R17 frame a microdomain in the alpha1 helix of dystrophin R17 for neuronal NOS binding. Proc. Natl. Acad. Sci. USA, 110, 525-530.
32. Johnson, E. K., Zhang, L., Adams, M. E., Phillips, A., Freitas, M. A., Froehner, S. C., Green-Church, K. B. and Montanaro, F. (2012) Proteomic analysis reveals new cardiac-specific dystrophin-associated proteins. PLoS One, 7, e43515.
33. Lai, Y., Thomas, G. D., Yue, Y., Yang, H. T., Li, D., Long, C., Judge, L., Bostick, B., Chamberlain, J. S., Terjung, R. L. and Duan, D. (2009) Dystrophins carrying spectrin-like repeats 16 and 17 anchor nNOS to the sarcolemma and enhance exercise performance in a mouse model of muscular dystrophy. J. Clin. Invest., 119, 624-635.
34. Bennett, V. and Lorenzo, D. N. (2016) An Adaptable Spectrin/Ankyrin-Based Mechanism for Long-Range Organization of Plasma Membranes in Vertebrate Tissues. Curr Top Membr, 77, 143-184.
35. Yoshida, M., Hama, H., Ishikawa-Sakurai, M., Imamura, M. Mizuno, Y., Araishi, K., Wakabayashi-Takai, E., Noguchi, S., Sasaoka, T. and Ozawa, E. (2000) Biochemical evidence for association of dystrobrevin with the sarcoglycan-sarcospan complex as a basis for understanding sarcoglycanopathy. Hum. Mol. Genet., 9, 1033-1040.
36. Suzuki, A., Yoshida, M. and Ozawa, E. (1995) Mammalian alpha 1- and beta 1-syntrophin bind to the alternative splice-prone region of the dystrophin COOH terminus. J. Cell Biol., 128, 373-381.
37. Yang, B., Jung, D., Rafael, J. A., Chamberlain, J. S. and Campbell, K. P. (1995) Identification of alpha-syntrophin binding to syntrophin triplet, dystrophin, and utrophin. J. Biol. Chem., 270, 4975-4978.
38. Bunnell, T. M., Jaeger, M. A., Fitzsimons, D. P., Prins, K. W. and Ervasti, J. M. (2008) Destabilization of the dystrophin-glycoprotein complex without functional deficits in alpha-dystrobrevin null muscle. PLoS One, 3, e2604.
39. Bajanca, F., Gonzalez-Perez, V., Gillespie, S. J., Beley, C. Garcia, L., Theveneau, E., Sear, R. P. and Hughes, S. M. (2015) In vivo dynamics of skeletal muscle Dystrophin in zebrafish embryos revealed by improved FRAP analysis. Elife, 4, e06541.
40. Shin, J. H., Yue, Y. and Duan, D. (2012) Recombinant adeno-associated viral vector production and purification. Methods Mol. Biol., 798, 267-284.
41. Zhong, L., Li, B., Mah, C. S., Govindasamy, L., Agbandje-McKenna, M., Cooper, M., Herzog, R. W., Zolotukhin, I., Warrington, K. H. J., Weigel-Van Aken, K. A. et al. (2008) Next generation of adeno-associated virus 2 vectors: point mutations in tyrosines lead to high-efficiency transduction at lower doses. Proc. Natl. Acad. Sci. USA, 105, 7827-7832.
42. Shin, J. H., Yue, Y. Srivastava, A., Smith, B., Lai, Y. and Duan, D. (2012) A Simplified Immune Suppression Scheme Leads to Persistent Micro-dystrophin Expression in Duchenne Muscular Dystrophy Dogs. Hum. Gene Ther., 23, 202-209.
43. Liadaki, K., Luth, E. S. and Kunkell, L. M. (2007) Co-detection of GFP and dystrophin in skeletal muscle tissue sections. BioTechniques, 42, 699-700.
44. Lai, Y., Zhao, J., Yue, Y., Wasala, N. B. and Duan, D. (2014) Partial restoration of cardiac function with ΔPDZ nNOS in aged mdx model of Duchenne cardiomyopathy. Hum. Mol. Genet., 23, 3189-3199.

### SUPPLEMENTARY REFERENCES

1. Suzuki, A., Yoshida, M., Yamamoto, H. and Ozawa, E. (1992) Glycoprotein-binding site of dystrophin is confined to the cysteine-rich domain and the first half of the carboxy-terminal domain. FEBS Lett., 308, 154-160.
2. Suzuki, A., Yoshida, M., Hayashi, K., Mizuno, Y., Hagiwara, Y. and Ozawa, E. (1994) Molecular organization at the glycoprotein-complex-binding site of dystrophin. Three dystrophin-associated proteins bind directly to the carboxy-terminal portion of dystrophin. Eur. J. Biochem., 220, 283-292.
3. Campbell, K. P. and Kahl, S. D. (1989) Association of dystrophin and an integral membrane glycoprotein. Nature, 338, 259-262.
4. Jung, D., Yang, B., Meyer, J., Chamberlain, J. S. and Campbell, K. P. (1995) Identification and characterization of the dystrophin anchoring site on beta-dystroglycan. J. Biol. Chem., 270, 27305-27310.
5. Huang, X., Poy, F., Zhang, R., Joachimiak, A., Sudol, M. and Eck, M. J. (2000) Structure of a WW domain containing fragment of dystrophin in complex with beta-dystroglycan. Nat. Struct. Biol., 7, 634-638.
6. Ishikawa-Sakurai, M., Yoshida, M., Imamura, M., Davies, K. E. and Ozawa, E. (2004) ZZ domain is essentially required for the physiological binding of dystrophin and utrophin to beta-dystroglycan. Hum. Mol. Genet., 13, 693-702.
7. Draviam, R. A., Wang, B., Li, J., Xiao, X. and Watkins, S. C. (2006) Mini-dystrophin efficiently incorporates into the dystrophin protein complex in living cells. J Muscle Res Cell Motil, 27, 53-67.
8. Einbond, A. and Sudol, M. (1996) Towards prediction of cognate complexes between the WW domain and proline-rich ligands. FEBS Lett., 384, 1-8.
9. Recan, D., Chafey, P., Leturcq, F., Hugnot, J. P., Vincent, N., Tome, F Collin, H., Simon, D., Czernichow, P., Nicholson, L. V. and et, A. (1992) Are cysteine-rich and COOH-terminal domains of dystrophin critical for sarcolemmal localization? J. Clin. Invest., 89, 712-716.
10. Hoffman, E. P Garcia, C. A Chamberlain, J. S., Angelini, C., Lupski, J. R. and Fenwick, R. (1991) Is the carboxyl-terminus of dystrophin required for membrane association? A novel, severe case of Duchenne muscular dystrophy. Ann. Neurol., 30, 605-610.
11. Helliwell, T. R., Ellis, J. M Mountford, R. C., Appleton, R. E. and Morris, G. E. (1992) A truncated dystrophin lacking the C-terminal domains is localized at the muscle membrane. Am. J. Hum. Genet., 50, 508-514.
12. Rafael, J. A., Cox, G. A., Corrado, K., Jung, D Campbell, K. P. and Chamberlain, J. S. (1996) Forced expression of dystrophin deletion constructs reveals structure-function correlations. J. Cell Biol., 134, 93-102.
13. Maconochie, M. K., Simpkins, A. H., Damien, E., Coulton, G., Greenfield, A. J. and Brown, S. D. (1996) The cysteine-rich and C-terminal domains of dystrophin are not required for normal costameric localization in the mouse. Transgenic Res, 5, 123-130.
14. Gardner, K. L., Keamey, J. A Edwards, J. D. and Rafael-Fortney, J. A. (2006) Restoration of all dystrophin protein interactions by functional domains in trans does not rescue dystrophy. Gene Ther, 13, 744-751.
15. Bamabei, M. S Sjaastad, F. V., Townsend, D., Bedada, F. B. and Metzger, J. M. (2015) Severe dystrophic cardiomyopathy caused by the enteroviral protease 2A-mediated C-terminal dystrophin cleavage fragment. Sci Transl Med, 7, 294ra106.
16. Dunckley, M. G Wells, K. E., Piper, T. A., Wells, D. J. and Dickson, G. (1994) Independent localization of dystrophin N- and C-terminal regions to the sarcolemma of mdx mouse myofibres in vivo. J. Cell Sci., 107, 1469-1475.
17. Fritz, J. D., Danko, I., Roberds, S. L., Campbell, K. P., Latendresse, J. S. and Wolff, J. A. (1995) Expression of deletion-containing dystrophins in mdx muscle: implications for gene therapy and dystrophin function. Pediatr Res, 37, 693-700.
18. Sarkis, J., Hubert, J. F., Legrand, B., Robert, E., Cheron, A., Jardin, J., Hitti, E., Le Rumeur, E. and Vie, V. (2011) Spectrin-like repeats 11-15 of human dystrophin show adaptations to a lipidic environment. J. Biol. Chem., 286, 30481-30491.
19. Legardinier, S., Raguenes-Nicol, C Tascon, C Rocher, C Hardy, S., Hubert, J. F. and Le Rumeur, E. (2009) Mapping of the lipid-binding and stability properties of the central rod domain of human dystrophin. J. Mol. Biol., 389, 546-558.
20. Legardinier, S., Hubert, J. F., Le Bihan, O., Tascon, C., Rocher, C., Raguenes-Nicol, C., Bondon, A., Hardy, S. and Le Rumeur, E. (2008) Sub-domains of the dystrophin rod domain display contrasting lipid-binding and stability properties. Biochim. Biophys. Acta, 1784, 672-682.
21. Le Rumeur, E., Pottier, S., Da Costa, G Metzinger, L., Mouret, L., Rocher, C., Fourage, M., Rondeau-Mouro, C. and Bondon, A. (2007) Binding of the dystrophin second repeat to membrane di-oleyl phospholipids is dependent upon lipid packing. Biochim. Biophys. Acta, 1768, 648-654.
22. Hir, S. A., Raguenes-Nicol, C Paboeuf, G., Nicolas, A., Le Rumeur, E. and Vie, V. (2014) Cholesterol favors the anchorage of human dystrophin repeats 16 to 21 in membrane at physiological surface pressure. Biochim. Biophys. Acta, 1838, 1266-1273.
23. Le Rumeur, E Fichou, Y., Pottier, S., Gaboriau, F., Rondeau-Mouro, C., Vincent, M., Gallay, J. and Bondon, A. (2003) Interaction of dystrophin rod domain with membrane phospholipids. Evidence of a close proximity between tryptophan residues and lipids. J. Biol. Chem., 278, 5993-6001.
24. Suzuki, A., Yoshida, M. and Ozawa, E. (1995) Mammalian alpha 1- and beta 1-syntrophin bind to the alternative splice-prone region of the dystrophin COOH terminus. J. Cell Biol., 128, 373-381.
25. Yang, B., Jung, D., Rafael, J. A., Chamberlain, J. S. and Campbell, K. P. (1995) Identification of alpha-syntrophin binding to syntrophin triplet, dystrophin, and utrophin. J. Biol. Chem., 270, 4975-4978.
26. Yoshida, M., Hama, H., Ishikawa-Sakurai, M., Imamura, M Mizuno, Y., Araishi, K., Wakabayashi-Takai, E., Noguchi, S., Sasaoka, T. and Ozawa, E. (2000) Biochemical evidence for association of dystrobrevin with the sarcoglycan-sarcospan complex as a basis for understanding sarcoglycanopathy. Hum. Mol. Genet., 9, 1033-1040.
27. Cox, G. A., Sunada, Y., Campbell, K. P. and Chamberlain, J. S. (1994) Dp71 can restore the dystrophin-associated glycoprotein complex in muscle but fails to prevent dystrophy. Nat. Genet., 8, 333-339.
28. Rapaport, D Greenberg, D. S., Tal, M., Yaffe, D. and Nudel, U. (1993) Dp71, the nonmuscle product of the Duchenne muscular dystrophy gene is associated with the cell membrane. FEBS Lett., 328, 197-202.
29. Judge, L. M., Haraguchiln, M. and Chamberlain, J. S. (2006) Dissecting the signaling and mechanical functions of the dystrophin-glycoprotein complex J. Cell Sci., 119, 1537-1546.

The inclusion of various references herein is not to be construed as any admission by the Applicant that the references constitute prior art. Applicants expressly reserve their right to challenge any allegations of unpatentability of inventions disclosed herein over the references included herein.

Having illustrated and described the principles of the present disclosure, it should be apparent to persons skilled in the art that the disclosure can be modified in arrangement and detail without departing from such principles.

Although the materials and methods of this disclosure have been described in terms of various embodiments and illustrative examples, it will be apparent to those of skill in the art that variations can be applied to the materials and methods described herein without departing from the concept, spirit and scope of the disclosure. All such similar substitutes and modifications apparent to those skilled in the art are deemed to be within the spirit, scope and concept of the disclosure as defined by the appended claims or otherwise disclosed herein.

1. A synthetic nucleic acid molecule encoding a synthetic mini-dystrophin gene or micro-dystrophin gene encoding a synthetic, non-full length dystrophin protein comprising:
(i) an N-terminal (NT) domain of the dystrophin protein or a modified N-terminal domain of the dystrophin protein; and
(ii) an MBM of an R11-R12 MBD, wherein the MBM of R11-R12 comprises a cysteine residue corresponding to the conserved cysteine residue of the S-palmitoylation site peptide of SEQ ID NO: 57;
wherein the domains and the MBM are arranged from N to C terminus in the order in which they occur in a wild-type dystrophin protein and are operably linked.

2. The synthetic nucleic acid molecule of claim 1, wherein at least one domain is operably linked to an MBM of an R1-R2, wherein the MBM of R1-R2 comprises a cysteine residue corresponding to the conserved cysteine residue of the S-palmitoylation site peptide of SEQ ID NO: 54.

3. The synthetic nucleic acid of claim 1, wherein at least one domain is operably linked to an MBM of a CR membrane binding domain.

4. The synthetic nucleic acid of claim 1, wherein at least one domain is operably linked to an MBM of a CT membrane binding domain.

5. The synthetic nucleic acid of claim 1, wherein at least one domain and at least one domain and at least one other domain are operably linked with a hinge region selected from the group consisting of a synthetic hinge, a semi-synthetic hinge, dystrophin H1, dystrophin H2, dystrophin H3, and dystrophin H4.

6. The synthetic nucleic acid of claim 5 wherein the hinge region is independently selected from the group consisting of:
(i) the dystrophin H1 hinge or a variant thereof operably links the C-terminus of the NT domain to the N-terminus of an MBM or domain containing an MBM; or
(ii) the dystrophin H2 hinge or a variant thereof operably links the C-terminus of the NT domain or the C-terminus of an MBM or domain containing an MBM to the N-terminus of another MBM or domain containing another MBM; or
(iii) the dystrophin H3 hinge or a variant thereof operably links the C-terminus of the NT domain or the C-terminus of an MBM or domain containing an MBM to the N-terminus of another MBM or domain containing another MBM; or
(iv) the dystrophin H4 hinge or a variant thereof operably links the C-terminus of an MBM to the N-terminus of the CR MBM or the CR domain, or any combination thereof.

7. The synthetic nucleic acid molecule of claim 1, wherein: (i) the mini- or micro-dystrophin gene is between 5 kb to about 8 kb in length or less than 5 kb in length, respectively; or (ii) the mini- or micro-dystrophin gene is operably linked to a heterologous promoter, a heterologous 5′ untranslated region (UTR), a heterologous 3′ UTR, a heterologous polyadenylation site, or any combination thereof; or (iii) a full-length or near-full-length dystrophin gene of 8 kb to 12 kb is operably linked to a heterologous promoter, a heterologous 5′ untranslated region (UTR), a heterologous 3′ UTR, a heterologous polyadenylation site, or any combination thereof.

8. The synthetic nucleic acid molecule of claim 1, wherein said molecule is integrated within an endogenous dystrophin gene locus in an X-chromosome.

9. A lentiviral vector comprising the synthetic nucleic acid molecule of claim 1, wherein the nucleic acid molecule is operably linked to an expression cassette, 5′ and 3′ long terminal repeats (LTR), and a psi sequence in the lentiviral vector.

10. A single recombinant adeno-associated virus (AAV) vector comprising the nucleic acid of claim 1, wherein said nucleic acid molecule is operably linked to an expression cassette and viral inverted terminal repeats (ITRs) in the AAV.

11. A dual recombinant AAV vector system, comprising two AAV vectors, wherein one of the two AAV vectors comprises a part of the nucleic acid molecule of claim 1, and the other vector comprises the remaining part of said nucleic acid molecule, wherein the two vectors further comprise sequences that permit recombination with each other to produce said nucleic acid in full length, and wherein the nucleic acid in full length is operably linked to an expression cassette and viral ITRs.

12. A tri-recombinant AAV vector system, comprising three AAV vectors, wherein one of the three AAV vectors comprises a part of the nucleic acid molecule of claim 1, and the other two vectors comprise the remaining part of said nucleic acid molecule, wherein the three vectors further comprise sequences that permit recombination with each other to produce said nucleic acid in full length, and wherein the nucleic acid in full length is operably linked to an expression cassette and viral ITRs.

13. A composition comprising the synthetic nucleic acid molecule of claim 1 and a pharmaceutically acceptable carrier.

14. The composition of claim 13, wherein: (i) the nucleic acid molecule is operably linked to an expression cassette, 5′ and 3′ long terminal repeats (LTR), and a psi sequence in a lentiviral vector; or (ii) the nucleic acid molecule is operably linked to an expression cassette and viral inverted terminal repeats (ITRs) in an AAV.

15. An isolated host cell comprising the synthetic nucleic acid molecule of claim 1.

16. The host cell of claim 15, wherein said nucleic acid molecule is integrated within an endogenous dystrophin gene locus in a chromosome of the host cell.

17. The host cell of claim 15, wherein: (i) the nucleic acid molecule is operably linked to an expression cassette, 5′ and 3′ long terminal repeats (LTR), and a psi element in a lentiviral vector: or (ii) said nucleic acid molecule is operably linked to an expression cassette and ITRs in an AAV.

18. The host cell of claim 15, wherein the host cell is a myogenic stem cell.

19. A method for the treating or ameliorating one or more adverse effects of Duchenne muscular dystrophy (DMD), Becker muscular dystrophy (BMD) or X-linked dilated cardiomyopathy (XLDC) in a subject in need thereof comprising the step of administering to the subject a therapeutically effective amount of the synthetic nucleic acid molecule of claim 1.

20. The method of claim 19, wherein the administration is by injection into muscle or systemic delivery.

21. The method of claim 19, wherein the synthetic nucleic acid is administered via an isolated host cell comprising said synthetic nucleic acid molecule and wherein the host cell is a stem cell or myogenic stem cell.

22. The method of claim 19, wherein the synthetic nucleic acid is administered via an isolated host cell comprising said synthetic nucleic acid molecule and wherein the host cell is derived from an autologous cell of the subject.

23. The method of claim 19, wherein a defective endogenous dystrophin gene of the host cell or a defective portion thereof is edited to provide the synthetic nucleic acid molecule within the host cell's X-chromosome.

24. The method of claim 19, wherein the synthetic nucleic acid molecule is administered via a lentiviral vector comprising the synthetic nucleic acid molecule, and wherein the nucleic acid molecule is operably linked to an expression cassette, 5′ and 3′ long terminal repeats (LTR), and a psi sequence in the lentiviral vector.

25. The method of claim 19, wherein the synthetic nucleic acid molecule is administered via a single recombinant adeno-associated virus (AAV) vector comprising said synthetic nucleic acid molecule, and wherein said nucleic acid molecule is operably linked to an expression cassette and viral inverted terminal repeats (ITRs) in the AAV.
